(19) United States
(12) Patent Application Publication
(10) Pub. No.: US 2026/0364168 A1
(43) Pub. Date: Nov. 19, 2026

(54) USE OF PROTEIN KINASE C (PKC) ACTIVATING COMPOUNDS AS AN ADJUVANT

(71) Applicants: Ruihong CHEN, San Mateo (CA); Chun JIANG, San Mateo, CA (US); K-GEN THERAPEUTICS, INC., Burlingame, CA (US)

(72) Inventors: Chun JIANG, Hillsborough, CA (US); Ruihong CHEN, Burlingame, CA (US)

(73) Assignee: K-GEN THERAPEUTICS, INC., San Mateo, CA (US)

(21) Appl. No.: 19/114,386

(22) PCT Filed: Sep. 22, 2023
(86) PCT No.: PCT/US2023/033552

§ 371 Date: Mar. 21, 2025

Related U.S. Application Data
(60) Provisional application No. 63/376,603, filed on Sep. 22, 2022.

(51) Int. Cl. A61K 39/39; A61K 39/00; A61K 39/215; A61P 37/04

(52) U.S. Cl. CPC A61K 39/39; A61K 39/215; A61P 37/04; A61K 2039/53; A61K 2039/55511

(57) ABSTRACT

This present disclosure relates to a method of generating an immune response using an antigenic material in combination with compounds that activate protein kinase C (PKC) as an adjuvant, particularly in vaccination against pathogenic agents.

### 1. CROSS-REFERENCE TO RELATED APPLICATIONS

This application claims the benefit of U.S. Provisional Patent Application No. 63/376,603, filed Sep. 22, 2022, which is incorporated herein by reference in its entirety.

### 2. BACKGROUND

Vaccination becomes increasingly important for public health in preventing serious and life-threatening diseases from pathogens such as COVID-19, Monkeypox, Influenza, RSV, AIDS, malaria, etc. Vaccine adjuvant can stimulate immune response to vaccines and increase vaccine efficacy. Adjuvant also helps the vaccine to achieve the same efficacy with reduced vaccine dosage/frequency, minimizing any potential side effects. Thus, new adjuvants that can improve immunogenicity and efficacy of vaccines are urgently needed.

Recent reports “Emerging concepts in the science of vaccine adjuvants” (Pulendran et al., Rev Drug Discov., 2021, 20(6):454) and “Inflammasome-mediated immunogenicity of clinical and experimental vaccine adjuvants” (Reinke et al., Vaccines (Basel), 2020, 8(3):554) reviewed different classes and modes of action of current clinically approved and new experimental vaccine adjuvants.

Aluminum salts (alum) are the oldest vaccine adjuvants included in vaccines against hepatitis B, diphtheria, tetanus and pertussis or human papilloma virus. MF59, an oil-in-water emulsion adjuvant (Squalene/Tween 80/Span 85), has been developed and approved in an influenza vaccine in Europe. These oil-in-water emulsions can help antigen release for an extended period of time and attract innate immune cells to pick up the antigens. A few other newer adjuvants and adjuvant combinations have been developed and included in vaccine products. For example, AS04 consists of MPL (a detoxified form of lipopolysaccharide LPS) adsorbed on alum, which is included in vaccine products such as the hepatitis B vaccine and the human papilloma vaccine. MPL, adsorbed on alum, is still capable of activating TLR4, thus AS04 elicits its adjuvant effect by combination effect of alum and TLR4 signaling on innate immune cells. AS03 (Squalence/α-tocopherol (vitamin E)/Tween 80) is similar to MF59 oil-in-water adjuvant but with an addition of vitamin E in the emulsion. AS03 is included in the pandemic influenza vaccines. Like other adjuvants, AS03 triggers innate immune responses and modulates production of cytokines and chemokines such as IL-6, G-CSF (granulocyte colony-stimulating factor), CCL2, and CCL3. It also facilitates antigen processing and presentation in dendritic cells (DCs), monocytes and neutrophils. AS01 consists of two well-known immunostimulatory components in liposomes: 1) MPL, the TLR4 agonist used in AS04 and 2) QS-21, a purified saponin (triterpene glycosides). QS-21 activates caspase 1, possibly via NLRP3 inflammasomes. AS02 is an oil-in-water adjuvant containing the same two immunostimulatory components as in AS01. Synergistic activation of two different innate pathways is critical for greater adaptive immune response and the full adjuvant effect of AS01 and AS02. AS01 is included in the shingles and malaria vaccines. Cytosine phosphoguanine (CpG) 1018 is a 22-mer single-stranded oligonucleotide, which is a TLR9 agonist capable of stimulating MyD88 pathway and type I interferon response in plasmacytoid DCs. It also stimulates strong B cell activation. It is the adjuvant for the hepatitis B vaccine Heplisav-B, an improved two-dose regimen hepatitis B vaccine.

Adjuvants enhance immunogenicity of antigens in many ways. Adjuvants can prolong the duration of antigen release, recruit innate immune cells including antigen presenting cells (APCs) to the injection site, enhance differentiation and maturation of APCs, stimulate antigen uptake by APCs, or activate inflammasomes and/or Toll-like receptors (TLRs) leading to activation of NFκB signaling and production and secretion of interferons, cytokines and chemokines. Activation/modulation of the innate immune systems by adjuvants leads to improved adaptive immunity.

Aluminum salts, saponins, and many commonly used adjuvants in clinic are activators of inflammasomes (e.g. NLRP3 inflammasome) in the innate immune system. These vaccine adjuvants promote immunogenicity through inflammasome-mediated signaling. Activation of the inflammasome complexes and caspases result in the processing and secretion of pro-inflammatory cytokines of the IL-1 family, which can in turn stimulate immune activation of humoral and cellular adaptive responses for long-lasting immunity. Thus, NLRP3 inflammasomes represent valuable targets of vaccine adjuvants. PKD (PKD1 is PKCμ) signaling has been shown to be required and sufficient for the activation of NLRP3 inflammasome (Zhang et al., J Exp Med., 2017; 214(9): 2671-2693). Agents or compounds that activate PKD could be potential vaccine adjuvant candidates.

TLR signaling pathways are critical for many vaccine adjuvants to elicit their functions. As described above, AS01, AS02, and AS04 contain MPL, a TLR4 agonist, whereas CpG 1018 is a TLR9 agonist. Agents activating other TLRs, such as TLR3 agonist dsRNA Poly (I:C), TLR5 agonist flagellin, TLR7/8 agonist imiquimod or resiquimod, have been tested as potential adjuvants. Many TLR agonists exert their effects via MyD88 pathway leading to activation of NFκB which induces expression of pro-inflammatory cytokines. Interestingly, MyD88 has been found to serve as the link coupling TLRs to PKCε recruitment and activation that is required for TLR-induced NFκB activation (Faisal et al., J Biol Chem., 2008, 283(27):18591-600). PKD1 (PKCμ) can also be activated by many TLR ligands in a MyD88-dependent manner and it is essential for MyD88-dependent pro-inflammatory immune responses (Park et al., J Immunol., 2009, 182 (10):6316-6327). In addition to PKCε and PKD1 (PKCμ), PKCα, a conventional PKC isoform, is also involved in MyD88-dependent TLR signaling and cytokine production in DCs (Langlet et al., Eur J Immunol., 2010, 40(2):505-15). Moreover, Phorbol 12-myristate 13-acetate (PMA), a pan-PKC activator, has been reported to induce PKC-dependent NFκB activation in both TLR5-dependent and -independent manner in vitro, and boosts antigen-specific antibody as well as T cell responses in vivo (Oh et al., Mediators Inflamm., 2014, 2014:406514).

Some other adjuvant targets include retinoic acid-inducible gene I (RG-1), activated by short RNA, melanoma differentiation-associated protein 5 (MDA5), activated by long RNA, and stimulator of interferon genes (STING), activated by dsDNA. Activation of transcription factors IRF7/IRF3 by RIG-I and MDA5 leads to production of type I interferons whereas activation of IRF3 and NFκB by STING stimulates type I interferons and pro-inflammatory cytokines. Interestingly, TLR3, activated by dsRNA, can stimulate type I interferon production via TIR domain-containing adapter-inducing IFNβ (TRIF) and IRF3. Again, PKCα has been found to be involved in IRF3 activation and type I interferon synthesis (Johnson et al., J Biol Chem., 2007, 282(20):15022-32). Furthermore, as an adaptor protein in the cytoplasm of host mammalian cells, STING activates a TANK binding kinase (TBK1)-IRF3-NFκB signaling axis, resulting in the induction of IFNβ amd other gene products that strongly activate innate immunity. Thus, STING is understood to be a component of a host cytosolic surveillance pathway (Vance et al., (2009)) that senses infection with intracellular pathogens such as viruses and, in response, induces the production of IFN-3, leading to the development of an adaptive protective pathogen-specific immune response consisting of both antigen-specific CD4 and CD8 T cells as well as pathogen-specific antibodies. In addition to its role in innate immunity and an antiviral response, TBK1 also regulates cell proliferation, apoptosis, autophagy, and anti-tumor immunity. However, insufficient regulation of TBK1 activity leads to autoimmune, neurodegenerative diseases or tumorigenesis.

A rational combination of adjuvants can provide greater potency while maintaining a good safety profile. The AS0 series of adjuvants, successfully used in many vaccines, contain multiple components targeting more than one adjuvant target and exerting their adjuvant effects by multiple mechanisms. For example, AS01 and AS02 contain a combination of a NLRP3-activating agent and a TLR agonist, where the TLR agonist induces upregulation of pro-inflammatory genes to enhance a more robust innate immune responses than the NLRP3-activating agent alone, resulting in greater adaptive immune responses. Therefore, combinatorial adjuvants could serve as a guidance for developing agents affecting multiple adjuvant targets/nodes.

Improved understanding of mechanisms and pathways as well as immune cell populations targeted by current adjuvants may aid in the design of better adjuvants for use in vaccines. Most current vaccine adjuvants activate various signaling pathways directly (e.g. TLRs and NLRP3 inflammasome) in innate immune cells (e.g. DCs, monocytes, macrophages, and neutrophils). Subsequent activation of NFκB and other transcription factors (e.g. IRF3) leads to production and secretion of soluble cytokines, chemokines, and interferons as well as ensuing adaptive immune responses. Beyond the current paradigm of how adjuvants function, modalities targeting both innate and adaptive immune cells simultaneously may have advantages over those direct stimulators of only innate immune cells.

Adjuvant candidates that stimulate immune activation of several innate pathways and adaptive immune systems simultaneously could induce more robust immune responses (both humoral and cellular adaptive immune responses), thus having a great potential to be successful in the clinic, particularly in patients with weaker immune systems.

### 3. SUMMARY

The present disclosure provides use of PKC activating compounds as adjuvants for enhancing an immune response to an antigenic material. In some embodiments, provided herein is a method of generating an immune response, comprising administering to a subject an antigenic material and an adjuvant comprising an effective amount of a PKC activating compound, with the proviso that the antigenic material is not a cancer vaccine or cancer antigen.

In some embodiments, the antigenic material is, or is from, a viral, bacterial, fungal or protozoal pathogen. In some embodiments, the antigenic material is a virus, bacterium, fungus or protozoan. In some embodiments, the virus, bacteria, fungi or protozoa are attenuated but live virus, bacteria, fungi, or protozoa. The live virus, bacteria, fungi, or protozoa are sufficiently attenuated to eliminate or reduce the occurrence of an infection when administered as a vaccine.

In some embodiments, the antigenic material comprises an antigen prepared or isolated from the viral, bacterial, fungal, or protozoal organism. Such antigenic material can be in the form of a protein, glycoprotein, or antigenic oligosaccharide present on the virus, bacterium, fungus, or protozoan. In some embodiments, the antigenic material is a synthetically produced antigenic material, wherein the synthetically produced antigenic material is expressed or present on the virus, bacterium, fungus, or protozoan. For example, the pan-fungal biomarker 1-3 beta-d-glucan (also known as (1→3)-β-D-Glucan, or “BDG”) is a polysaccharide composed of glucose monomers linked by beta 1-3 glucosidic bonds and is found in high abundance in cellulose-containing plants, and in the cell wall of many fungi. Because yeast-derived β-glucans (such as β-1,3/1,6-glucan derived from baker's yeast) have been reported to stimulate the immune system, they are commercially available and marketed as a dietary supplement. In some embodiments, the synthetically produced antigenic material is made by recombinant DNA technology. In some embodiments, the antigenic material is an mRNA vaccine, which is administered to a subject to express the encoded protein or antigenic material in the subject.

In some embodiments, the antigenic material is, or is from, a virus. In some embodiments, the antigen material is an attenuated virus, an inactivated virus, a viral protein, recombinant viral protein, or mRNA vaccine of a viral protein. In some embodiments, the antigenic material from a virus is a viral toxin. In some embodiments, the viral toxin is rotavirus NSP4.

In some embodiments, the antigenic material is, or is from, a bacterium. In some embodiments, the antigenic material is attenuated or killed bacteria. In some embodiments, the antigenic material comprises two or more bacterial strains or species. In some embodiments, the antigenic material is a conjugate of two or more bacterial proteins and/or bacterial polysaccharides.

In some embodiments, the antigenic material from bacteria is a bacterial toxin or subunit vaccine. In some embodiments, the antigenic material is an mRNA vaccine of a bacterial protein.

In some embodiments, the antigenic material is, or is from, a fungal pathogen. In some embodiments, the antigenic material is, or is from, pathogenic Coccidioides, Aspergillus, Histoplasma, Blastomyces, Paracoccidioides, Pneumocystis, or Cryptococcus. In some embodiments, the antigenic material is an mRNA vaccine of a fungal protein.

In some embodiments, the antigenic material is, or is from, a protozoal pathogen. In some embodiments, the antigenic material is, or is from, Eimeria, Theileria, Toxoplasma, Tritrichomonas, Giardia, Sarcocystis, Neospora, Leishmania, or Babesia. In some embodiments, the antigenic material comprises a carbohydrate/polysaccharide antigen of a protozoal pathogen. In some embodiments, the antigenic material is an mRNA vaccine of a protozoal protein.

In some embodiments, the PKC activating compound is administered prior to, concurrently with, or subsequent to administration of the antigenic material. In some embodiments, the PKC activating compound is administered concurrently with the antigenic material, either separately by the same or different administration route, or together as a single composition.

In some embodiments, the PKC activating compound is subsequently administered to the subject one or more times following an initial administration or vaccination with the PKC activating compound and antigenic material.

In some embodiments, the subsequent administration of the PKC activating compound is in conjunction with administration of a subsequent dose of antigenic material. In some embodiments, the subsequent administration of the PKC activating compound is done without a subsequent dose of antigenic material. In some embodiments, following initial vaccination with antigenic material and PKC activating compound, one or more doses of PKC activating compound is administered without antigenic material followed by a second vaccination with antigenic material, with or without the PKC activating compound.

In some embodiments, the PKC activator compound for use as an adjuvant is a diterpenoid PKC activator compound. In some embodiments, the compound is of formula (I):

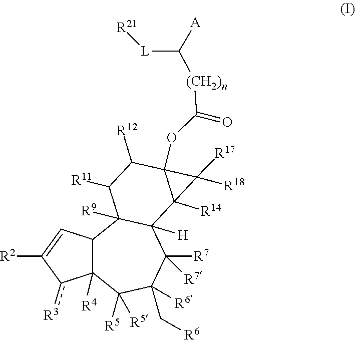

or a pharmaceutically acceptable salt, tautomer, or stereoisomer thereof;
   wherein
A is —OH, —C(O)OR′, or —NR¹³R^{13′};
R¹ is H or a M+ counterion;
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is

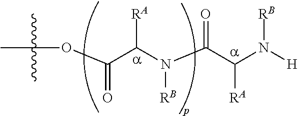

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R¹′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M+ counterion;
J¹ is selected from OH, CN, halo, C₁-C₄alkyl, and haloC₁-C₄alkyl; and
n is 0 or 1.

In some embodiments, A is —OH. In some embodiments, A is —C(O)OR¹, wherein R¹ is H or a M⁺ counterion. In some embodiments, A is —NR¹³R^{13′}, wherein R¹³ and R^{13′} are each independently H or C₁-C₄alkyl.

In some embodiments, the compound is of formula (II):

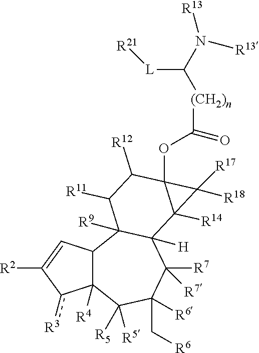

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

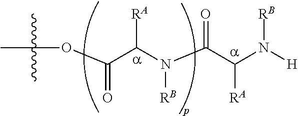

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₋₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M+ counterion;
J¹ is selected from OH, CN, halo, C₁-C₄alkyl, and haloC₁-C₄alkyl; and
n is 0 or 1.

In some embodiments, the compound is of formula (IIc):

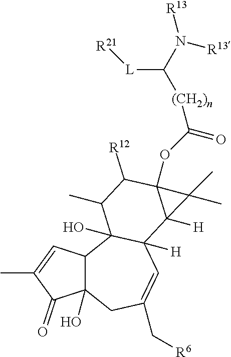

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

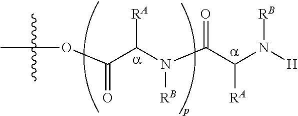

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R¹ is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
   R^{k} is H or M⁺ counterion;
   J¹ is selected from OH, CN, halo, C₁-C₄alkyl, and haloC₁-C₄alkyl; and
   n is 0 or 1.

In some embodiments, the compound is of formula (IV):

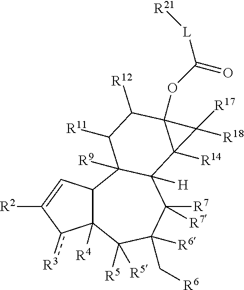

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

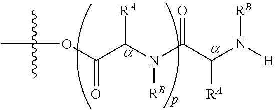

wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR^{g}; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₀alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

### 4. BRIEF DESCRIPTION OF THE DRAWINGS

FIG. 1 (Panels A-F) shows levels of various cytokines in peripheral blood mononuclear cell (PMBC) preparations treated with a PKC activating compound. Panel A: IFNγ; Panel B: GM-CSF; Panel C: IL-13; Panel D: IL-2; Panel E: TNF-α; and Panel F: IL-6. IFNγ, GM-CSF, and IL-13 expressions were strongly induced in a dose-dependent manner (up to >10-fold increase over DMSO) by the PKC activator compounds. Notations are as follows: PMA+Iono.=PMA 50 ng/mL+Ionomycin 1 g/mL; Resi.: Resiquimod (TLR 7/8); LPS=Lipopolysaccharides from Gram-negative bacteria outer membrane; K-47=K101-C1347; K-802=K101-C134802; and K-801=K101-C134801.

FIG. 2 illustrates one example of the effects of PKC activating compounds on SARS-CoV-2 Spike RBD-specific IgG endpoint titers in mice, as measured on day 28.

### 5. DETAILED DESCRIPTION

As used in this specification and the appended claims, the singular forms “a,” “an,” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

### 5.1. DEFINITIONS

In reference to the present disclosure, the technical and scientific terms used in the descriptions herein will have the meanings commonly understood by one of ordinary skill in the art, unless specifically defined otherwise. Accordingly, the following terms are intended to have the meanings as described below.

“Alkyl” refers to straight or branched chain hydrocarbon groups of 1 to 20 carbon atoms, particularly 1 to 12 carbon atoms (C₁-C₁₂ or C₁₋₁₂), and more particularly (C₁-C₈ or C₁₋₈) carbon atoms. Exemplary “alkyl” includes, but are not limited to, methyl, ethyl, n-propyl, i-propyl, n-butyl, s-butyl, t-butyl, n-pentyl, and s-pentyl.

“Alkenyl” refers to straight or branched chain hydrocarbon group of 2 to 20 carbon atoms, particularly 2 to 12 carbon atoms (C₂-C₁₂ or C₂₋₁₂), and most particularly 2 to 8 (C₂-C₈ or C₂₋₈)carbon atoms, having at least one double bond. Exemplary “alkenyl” includes, but are not limited to, vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkynyl” refers to a straight or branched chain hydrocarbon group of 2 to 12 carbon atoms (C₂-C₁₂ or C₂₋₁₂), particularly 2 to 8 carbon atoms (C₂-C₈ or C₂₋₈), containing at least one triple bond. Exemplary “alkynyl” includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkylene,” “alkenylene” and “alkynylene” refers to a straight or branched chain divalent hydrocarbon radical of the corresponding alkyl, alkenyl, and alkynyl, respectively. The “alkylene,” “alkenylene” and “alkynylene” may be optionally substituted, for example with alkyl, alkyloxy, hydroxyl, carbonyl, carboxyl, halo, nitro, and the like.

“Aliphatic” refers to an organic compound characterized by substituted or unsubstituted, straight or branched, and/or cyclic chain arrangements of constituent carbon atoms. Aliphatic compounds do not contain aromatic rings as part of the molecular structure of the compounds.

Aliphatic compound can have 1-20 (C₁-C₂₀ or C₁₋₂₀) carbon atoms, 1-12 (C₁-C₁₂ or C₁₋₁₂) carbon atoms, or particularly 1-8 (C₁-C₈ or C₁₋₈) carbon atoms.

“Lower” in reference to substituents refers to a group having between one and six carbon atoms.

“Cycloalkyl” refers to any stable monocyclic or polycyclic system which consists of carbon atoms, any ring of which being saturated. “Cycloalkenyl” refers to any stable monocyclic or polycyclic system which consists of carbon atoms, with at least one ring thereof being partially unsaturated. Examples of cycloalkyls include, but are not limited to, cyclopropyl, cyclobutyl, cyclopentyl, cyclohexyl, cycloheptyl, cyclooctyl, bicycloalkyls and tricycloalkyls (e.g., adamantyl).

“Heterocycloalkyl” or “heterocyclyl” refers to a substituted or unsubstituted 3 to 14 membered, mono- or bicyclic, non-aromatic hydrocarbon, wherein 1 to 3 carbon atoms a (e replaced by a heteroatom. Heteroatoms and/or heteroatomic groups which can replace the carbon atoms include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or lower alkyl. Examples include oxiranyl, oxetanyl, azetidynyl, oxazolyl, thiazolidinyl, thiazolyl, morpholinyl, pyrrolidinonyl, pyrrolidinyl, piperidinyl, piperazinyl, 2,3-dihydrofuranyl, dihydropyranyl, tetrahydrofuranyl, tetrahydropyranyl, dihydropyridinyl, tetrahydropyridinyl, tetrahydropyrimidinyl, tetrahydrothiophenyl, tetrahydrothiopyranyl, azapanyl, and the like.

“Carbocycle,” “carbocyclyl,” and “carbocyclic,” as used herein, refer to a non-aromatic saturated or unsaturated ring in which each atom of the ring is carbon. The ring may be monocyclic, bicyclic, tricyclic, or even of higher order. Thus, the terms “carbocycle,” “carbocyclyl,” and “carbocyclic” encompass fused, bridged and spirocyclic systems: Preferably a carbocycle ring contains from 3 to 14 atoms, including 3 to 8 or 5 to 7 atoms, such as for example, 6 atoms.

“Aryl” refers to a six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Examples of “aryl” groups include phenyl, naphthyl, indenyl, biphenyl, phenanthrenyl, naphthacenyl, and the like.

“Heteroaryl” refers to an aromatic heterocyclic ring, including both monocyclic and bicyclic ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur.

In some embodiments, the heteroaryl can be a 5 to 6 membered monocyclic, or 7 to 11 membered bicyclic ring systems. Examples of “heteroaryl” groups include pyrrolyl, pyrazolyl, imidazolyl, pyrazinyl, oxazolyl, isoxazolyl, thiazolyl, furyl, thienyl, pyridyl, pyrimidyl, benzoxazolyl, benzisoxazolyl, benzothiazolyl, purinyl, benzimidazolyl, indolyl, isoquinolyl, quinoxalinyl, quinolyl, and the like.

“Bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 5 to 12 ring members and 0-4 heteroatoms independently selected from nitrogen, oxygen, and sulfur. Such bridged bicyclic groups include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

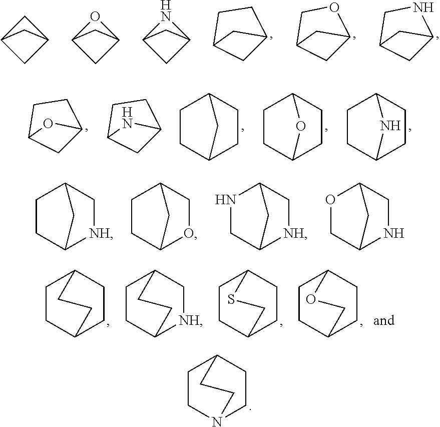

“Fused ring” refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Carbonyl” refers to —C(O)—. The carbonyl group may be further substituted with a variety of substituents to form different carbonyl groups including acids, acid halides, aldehydes, amides, esters, and ketones. For example, an —C(O)R′, wherein R′ is an alkyl is referred to as an alkylcarbonyl. In some embodiments, R′ is selected from an optionally substituted: alkyl, cycloalkyl, cycloalkylalkyl, heterocycloalkyl, heterocycloalkylalkyl, aryl, arylalkyl, heteroaryl, and heteroarylalkyl.

“Halogen” or “halo” refers to fluorine, chlorine, bromine and iodine.

“Haloalkyl” refers to an alkyl substituted with 1 or more halogen atoms. Preferably, the alkyl is substituted with 1 to 3 halogen atoms.

“Hydroxy” refers to —OH.

“Oxy” refers to group —O—, which may have various substituents to form different oxy groups, including ethers and esters. In some embodiments, the oxy group is an —OR′, wherein R′ is selected from an optionally substituted: alkyl, cycloalkyl, cycloalkylalkyl, heterocycloalkyl, heterocycloalkylalkyl, aryl, arylalkyl, heteroaryl, and heteroarylalkyl.

“Acyl” refers to —C(O)R′, where R is hydrogen, or an optionally substituted alkyl, heteroalkyl, cycloalkyl, heterocycloalkyl, heterocycloalkylalkyl, aryl, arylalkyl, heteroaryl, or heteroarylalkyl as defined herein. Exemplary acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to —OR′, wherein R′ is an optionally substituted alkyl.

“Aryloxy” refers to —OR′, wherein R′ is an optionally substituted aryl.

“Carboxy” refers to —COO— or COOM, wherein H or a M⁺ counterion.

“Carbamoyl” refers to —C(O)NR′R′, wherein each R′ is independently selected from H or an optionally substituted alkyl, cycloalkyl, cycloalkylalkyl, heterocycloalkyl, heterocylcoalkylalkyl, aryl, arylalkyl, heteroaryl, or heteroarylalkyl.

“Cyano” refers to —CN.

“Ester” refers to a group such as —C(═O)OR′, alternatively illustrated as —C(O)OR′, wherein R′ is selected from an optionally substituted: alkyl, cycloalkyl, cycloalkylalkyl, heterocycloalkyl, heterocyclolalkylalkyl, aryl, arylalkyl, heteroaryl, and heteroarylalkyl.

“Silyl” refers to Si, which may have various substituents, for example —SiR′R′R′, where R′ is as defined in the specification. For example, each R′ is independently selected from alkyl, cycloalkyl, cycloalkylalkyl, heterocyloalkyl, heterocycloalkylalkyl, aryl, arylalkyl, heteroaryl, and heteroarylalkyl. As defined herein, any heterocyloalkyl or heteroaryl group present in a silyl group has from 1 to 3 heteroatoms selected independently from O, N, and S.

“Thiol” refers to —SH.

“Sulfanyl” refers to —SR′, wherein R′ is selected from an optionally substituted: alkyl, cycloalkyl, cycloalkylalkyl, heterocyloalkyl, heterocycloalkylalkyl, aryl, arylalkyl, heteroaryl, and heteroarylalkyl. For example, —SR, wherein R is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R′, wherein R′ is an alkyl refers to an alkylsulfonyl. In some embodiments of —S(O)₂R′, R′ is selected from an optionally substituted: alkyl, cycloalkyl, cycloalkylalkyl, heterocyloalkyl, heterocycloalkylalkyl, aryl, arylalkyl, heteroaryl, and heteroarylalkyl.

“Amino” or “amine” refers to the group —NR′R′ or —NR′R′R′, wherein each R′ is independently selected from H and an optionally substituted: alkyl, cycloalkyl, heterocycloalkyl, alkyloxy, aryl, heteroaryl, heteroarylalkyl, acyl, alkyloxycarbonyl, sulfanyl, sulfinyl, sulfonyl, and the like. Exemplary amino groups include, but are not limited to, dimethylamino, diethylamino, trimethylammonium, triethylammonium, methylysulfonylamino, furanyl-oxy-sulfamino, and the like.

“Amide” refers to a group such as, —C(═O)NR′R′, wherein each R′ is independently selected from H and an optionally substituted: alkyl, cycloalkyl, cycloalkylalkyl, heterocycloalkyl, heterocycloalkylalkyl, aryl, arylalkyl, heteroaryl, and heteroarylalkyl.

“Spiroalkyl” as used herein refers to a monospiro compound having two alicyclic rings attached together through a single common carbon atom. In some embodiments, the spiro compounds have 5 to 12 total ring atoms (e.g., C₅-C₁₂ or C₅₋₁₂). In some embodiments, one or more of the carbon atoms can be replaced with a heteroatom, such as oxygen, nitrogen or sulfur. Exemplary spiroalkyl compounds include, among others, spiro[3,3]heptyl, spiro[3.4]octyl, and spiro[3,5]decyl.

“Adamantyl” refers to a compound of structural formula:

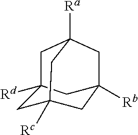

where optional substitutions can be present on one or more of R^{a}, R^{b}, R^{c}, and R^{d}. Adamantyl includes substituted adamantyl, e.g., 1- or 2-adamantyl, substituted by one or more substituents, including alkyl, halo, OH, NH₂, and alkoxy. Exemplary derivatives include methyladamatane, haloadamantane, hydroxyadamantane, and aminoadamantane (e.g., amantadine).

“N-protecting group” as used herein refers to those groups intended to protect a nitrogen atom against undesirable reactions during synthetic procedures. Exemplary N-protecting groups include, but is not limited to, acyl groups such acetyl and t-butylacetyl, pivaloyl, alkoxycarbonyl groups such as methyloxycarbonyl and t-butyloxycarbonyl (Boc), aryloxycarbonyl groups such as benzyloxycarbonyl (Cbz) and fluorenylmethoxycarbonyl (Fmoc and aroyl groups such as benzoyl. N-protecting groups are described in Greene's Protective Groups in Organic Synthesis, 5^{th} Edition, P. G. M. Wuts, ed., Wiley (2014).

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, OR′ (e.g., hydroxyl, alkyloxy (e.g., methoxy, ethoxy, and propoxy), aryloxy, heteroaryloxy, arylalkyloxy, ether, ester, carbamate, etc.), hydroxyalkyl, alkyloxycarbonyl, alkyloxyalkyloxy, perhaloalkyl, alkyloxyalkyl, SR (e.g., thiol, alkylthio, arylthio, heteroarylthio, arylalkylthio, etc.), S⁺R′₂, S(O)R′, SO₂R′, NR′R″ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, thiol, carboxy, aldehyde, keto, carboxylic acid, ester, amide, imine, and imide, including seleno and thio derivatives thereof, wherein each of the substituents can be optionally further substituted. In embodiments in which a functional group with an aromatic carbon ring is substituted, such substitutions will typically number less than about 10 substitutions, more preferably about 1 to 5, with about 1 or 2 substitutions being preferred.

“Stereoisomer” refers to a compound made up of the same atoms bonded by the same bonds but having different three-dimensional structures, which are not interchangeable. Thus, “stereoisomer thereof” with respect to a compound includes any stereoisomer of the compound and mixtures of stereoisomers, and includes “enantiomers,” which refers to two stereoisomers whose molecules are nonsuperimposable mirror images of one another. A compound may have more than one chiral center such that the compound may exist as either an individual diastereomer or as a mixture of diastereomers.

“Tautomer” refers to a proton shift from one atom of a molecule to another atom of the same molecule. Thus, “tautomers thereof” with respect to a compound includes any tautomers of the compound.

“Prodrug” refers to a derivative of an active compound (e.g., drug) that requires a transformation under the conditions of use, such as within the body or appropriate in vitro conditions, to release the active drug. Prodrugs are frequently, but not necessarily, pharmacologically inactive until converted into the active drug. Prodrugs can be obtained by masking a functional group in the drug believed to be in part required for activity with a progroup to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

Various progroups, as well as the resultant promoieties, suitable for masking functional groups in the active drugs to yield prodrugs can be used. For example, a hydroxyl functional group may be masked as a sulfonate, ester or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed, e.g., in vivo or under appropriate in vitro conditions, to provide the amino group. A carboxyl group may be masked as an ester (including silyl esters and thioesters), amide or hydrazide promoiety, which may be hydrolyzed in vivo to provide the carboxyl group. Included within the scope of prodrugs are, among others, “biohydrolyzable carbonate,” “biohydrolyzable ureide,” “biohydrolyzable carbamate,” “biohydrolyzable ester,” “biohydrolyzable amide,” and “biohydrolyzable phosphate” groups.

“Solvate” refers to a complex of variable stoichiometry formed by a solute, such as a PKC activator compound, and a solvent. Such solvents are selected to minimally interfere with the biological activity of the solute. Solvents may be, by way of example and not limitation, water, ethanol, or acetic acid.

“Hydrate” refers to a combination of water with a solute, such as a PKC activator compound, wherein the water retains its molecular state as water and is either absorbed, adsorbed or contained within a crystal lattice of the solute (e.g., PKC activating compound).

“Pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17^{th} Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” refers to an excipient, carrier or adjuvant that can be administered to a subject, together with at least one therapeutic agent, and which does not destroy the pharmacological activity thereof and is generally safe, nontoxic and neither biologically nor otherwise undesirable when administered in doses sufficient to deliver a therapeutic amount of the agent.

“Adjuvant” refers to a substance or combination of substances that enhances a subject's response to a therapeutic agent (e.g., by stimulating a stronger immune response in people receiving a vaccine). Thus, a vaccine adjuvant assists the vaccine, allowing it to achieve the same efficacy even when the dosage amount and/or frequency is reduced, thereby helping to minimize any potential side effects. Adjuvanted vaccines can, in some cases, cause more local reactions (such as redness, swelling, and pain at the injection site) and more systemic reactions (such as fever, chills and body aches) than non-adjuvanted vaccines. Some vaccines made from weakened or killed pathogens contain naturally occurring adjuvants and help the body produce a strong protective immune response.

“Subject” as used herein refers to a mammal, for example a dog, a cat, a horse, or a rabbit. In some embodiments, the subject is a non-human primate, for example a monkey, chimpanzee, or gorilla. In some embodiments, the subject is a human, sometimes referred to herein as a patient.

### 5.2. USES AND METHODS

PKC enzymes may be good adjuvant targets since they modulate both innate and adaptive immune responses (Isakov et al., Front Immunol., 2013, 4: 384). In addition to the involvement of a few PKC isoforms (PKD/PKCμ, PKCε, and PKCα) in the innate immunity describe above, PKCG is expressed in T cells and is required for T cell activation (Sun et al., Nature, 2000, 404:402-407; Anderson et al., Autoimmunity, 2006, 39(6):469-478). PKCG inhibits regulatory T cell-mediated immune suppression (Zanin-Zhorov et al., Science, 2010, 328:372). Other PKC isoforms such as PKCδ and PKCβ are involved in B cell functions (Mecklenbrauker et al., Nature, 2002, 416:860-865; Salzer et al., Blood, 2013, 121(16):3112-3116; Lim et al., Immunology, 2015, 146:508-522). Moreover, some PKC isoforms (e.g. PKC$) serve as important signaling nodes for differentiation and/or maturation of antigen presenting immune cells (e.g. DCs and macrophages) (Cejas et al., J Biol Chem., 2005, 280(31):28412; Tonetti et al., J Biol Chem., 1994, 269(37):23230). Compounds activating one or more PKC isoforms may be promising adjuvant candidates. Bryostatin, a PKC activator, induces chemokine expression in DC cells and stimulates an antigen-specific T cell responses from a peptide-based vaccine (Yan et al., Cytokine, 2010, 52(3):238).

Accordingly, in one aspect, the present disclosure provides a method of generating an immune response against an antigenic material by use of a PKC activator compound as an adjuvant, where the antigenic material is not a cancer vaccine or cancer antigen. In some embodiments, a method of generating an immune response comprises administering to a subject an antigenic material and an adjuvant comprising an effective amount of a PKC activating compound, with the proviso that the antigenic material is not a cancer vaccine or cancer antigen.

In some embodiments, the antigenic material is, or is from, a viral, bacterial, fungal or protozoal pathogen. In some embodiments, the antigenic material is a virus, bacterium, fungus or protozoan. In some embodiments, the virus, bacteria, fungi or protozoa are attenuated but live virus, bacteria, fungi, or protozoa that eliminates or reduces the occurrence of an infection when administered as a vaccine. In some embodiments, the antigenic material is a killed viral, bacterial, fungal, or protozoal preparation.

In some embodiments, the antigenic material comprises an antigen prepared or isolated from the viral, bacterial, fungal, or protozoal organism. These can be in the form of a protein, glycoprotein, or antigenic oligosaccharide present on the virus, bacteria, fungi, or protozoa. In some embodiments, the antigenic material is a synthetically produced antigenic material, wherein the synthetically produced antigenic material is expressed or present on the virus, bacteria, fungi, or protozoa. In some embodiments, the synthetically produced antigen material is made by recombinant DNA technology. In some embodiments, the antigenic material is an mRNA vaccine, which is administered to a subject to express the encoded protein or antigenic material in a subject.

In some embodiments, the antigenic material is, or is from, a virus. In some embodiments, the antigen material is an attenuated virus, an inactivated virus, a viral protein, recombinant viral protein, or mRNA vaccine of a viral protein. In some embodiments, the antigenic material from a virus is a viral toxin. In some embodiments, the viral toxin is rotavirus NSP4.

In some embodiments, the antigenic material is, or is from, a virus in the: Coronaviridae family, including e.g., the virus SARS-CoV-2 responsible for Coronavirus disease 2019 (COVID-19), Severe acute respiratory syndrome (SARS)-related coronavirus, and Middle East respiratory syndrome-related coronavirus (MERS-CoV); the Orthomyxoviridae family, including e.g., influenza viruses Influenza A virus (IAV), Influenza B virus (IBV), Influenza C virus (ICV), and Influenza D virus (IDV); the Papillomaviridae family, including human papilloma virus (HPV); the Pneumoviridae family, including respiratory syncytial virus (RSV) and human metapneumovirus (HMPV); the family Orthoherpesviridae includes, e.g., human herpesvirus 6A and 6B (HHV-6A and HHV-6B), human herpesvirus 7 (HHV-7), herpes simplex 1 and 2 (HSV-1 and HSV-2, also known as HHV-1 and HHV-2), human cytomegalovirus (HCMV also known as HHV-5), Epstein-Barr virus (EBV or HHV-4), varicella zoster virus (VZV or HHV-3), and the Rhadinovirus genus includes human herpesvirus 8 (HHV-8) or Kaposi's sarcoma-associated herpesvirus (KSHV, also known as HHV-8); the Filoviridae family, including Ebola virus, Marburg virus; the Rhabdoviridae family, including, e.g., rabies virus; the Hepadnaviridae family including, e.g., hepatitis B; the Picornaviridae family e.g., human rhinoviruses (HRVs), poliovirus, hepatitis A virus, coxsackieviruses; the Flaviviridae family, including e.g., hepatitis C virus (HCV) the West Nile virus, dengue virus, tick-borne encephalitis virus, yellow fever virus, Zika virus; the Deltaviridae family, e.g., hepatitis D virus (HDV); the family Retroviridae, e.g., human immunodeficiency virus (HIV) including HIV-1 and HIV-2, simian immunodeficiency virus, equine infectious anemia virus, bovine immunodeficiency virus, rabbit endogenous lentivirus type K (RELIK), e.g., an alpharetrovirus, such as Rous sarcoma virus; a betaretrovirus, such as simian retrovirus; a deltaretrovirus, such as bovine leukemia virus or human T-lymphotrophic virus (HTLV) including HTLV-1, HTLV-2, and HTLV-3; a gammaretrovirus, such as murine leukemia virus or feline leukemia virus; and feline immunodeficiency virus; the Paramyxoviridae family, including, e.g., human parainfluenza viruses (HPIVs), measles virus, mumps virus and Nipah virus (NiV); Adenoviridae family, e.g. human adenovirues (HAdVs); the Astroviridae family; the Matonaviridae family, including, e.g., rubella virus; the Caliciviridae family, including, e.g., norovirus and sapovirus; the Parvoviridae family, including, e.g., parvovirus B19, and human bocavirus 1; the Polyomaviridae family, e.g. BK polyomavirus (BKV); the Sedoreoviridae family, including rotavirus A and rotavirus B; the Togaviridae family, including Chikungunya virus (CHIKV); the Poxviridae family Orthopoxvirus genus including vaccinia virus, cowpox virus, smallpox virus, variola virus, and other animal pathogen poxviruses (e.g. monkeypox virus); or the Arenaviridae family, including the genus Mammarenavirus.

In some embodiments, the antigenic material is, or is from, a bacterium. In some embodiments, the antigenic material is a bacterial toxin. In some embodiments, the antigenic material and/or toxin is, or is from, a from a pathogenic bacterium family or genus including (but not limited to) Acinetobacter, Arcanobacterium, Bacillus (e.g., anthtrax), Bartonella, Bordetella, Borrelia (e.g., lyme disease), Brucella, Burkholderia, Campylobacter, Corynebacterium (e.g., diphtheria), Enterobacteriaceae, Enterococcus, Haemophilus, Heliobacteria, Legionella, Listeria, Neisseria meningitidis, Neisseria gonorrhoeae, Pneumococcus, Pseudomonas, Rickettsia, Salmonella, Shigella, Staphylococcus, Streptococcus, Escherichia, Klebsiella, Mycobacterium (e.g., tuberculosis), Salmonella enterica serovar Typhi, Chlamydia, Streptococcus pyogenes, Treponema, Vibrio (e.g., cholera), Yersinia, Clostridium or Clostridioides. In some embodiments, the antigenic material is an attenuated or killed bacterial vaccine.

In some embodiments, the antigenic material comprises two or more bacterial strains or species. In some embodiments, the two or more bacterial strains are conjugated, where the bacterial strains are inactivated. In some embodiments, the antigenic material is a conjugate of two or more bacterial proteins and/or bacterial polysaccharides.

In some embodiments, the antigenic material from bacteria is a bacterial toxin or subunit vaccines, particularly inactivated bacterial toxins. Example of toxins available in inactivated form include tetanus toxin, diphtheria toxin, and anthrax exotoxins.

In some embodiments, the antigenic material is, or is from, a fungal pathogen. In some embodiments, the antigenic material is, or is from, pathogenic Candida, Coccidioides, Aspergillus, Fusarium, Histoplasma, Blastomyces, Paracoccidioides, Pneumocystis, or Cryptococcus. In some embodiments, the antigenic material is an mRNA vaccine of a fungal protein.

In some embodiments, the antigenic material is a fungal protein or polysaccharide, for example, an invasion protein (agglutinin-like sequence 3 (Als3p), aspartyl proteinase-2, glucuronoxylomannan, Gel1p, Crf1p, Pep1p, Cat1p, Sod1p, Dpp5p, RNUp, Mep1p, 1-3 beta-d-glucan (also known as (1→3)-β-D-Glucan, or “BDG”), β-3/1,6-glucan, GM, glycolipids: GSL, LGM, β-glucans of S. cerevisiae, Adhesin BAD1, gp 43 (P10), C. posadasii Gel-1 (β 1,3, aspartyl proteinase), phospholipase B, or a mannosidase.

In some embodiments, the antigenic material is, or is from, a protozoal pathogen. In some embodiments, the antigenic material is, or is from, Eimeria, Plasmodium, Theileria, Toxoplasma, Tritrichomonas, Trypanosoma, Giardia, Sarcocystis, Neospora, Naegleria fowleri amoeba, Leishmania, or Babesia.

In some embodiments, the antigenic material comprises a carbohydrate/polysaccharide antigen of a protozoal pathogen.

In some embodiments, the PKC activating compound is used in conjunction with a commercially available vaccine, such as, for example: Adenovirus Type 4 and Type 7 Vaccine; Anthrax Vaccine Adsorbed, Adjuvanted (CYFENDUS®); Anthrax Vaccine Adsorbed (Biothrax®); BCG Vaccines (BCG Live and TICE® BCG); Cholera Vaccine (Vaxchora®); COVID-19 mRNA Vaccines (Comirnaty® and SPIKEVAX®); Dengue Tetravalent Vaccine, Live (DENGVAXIA®); Diphtheria & Tetanus Toxoids Adsorbed; Diphtheria & Tetanus Toxoids & Acellular Pertussis Vaccine Adsorbed (Infanrix® and DAPTACEL®); Diphtheria & Tetanus Toxoids & Acellular Pertussis Vaccine Adsorbed, Hepatitis B (recombinant) and Inactivated Poliovirus Vaccine Combined (Pediarix®); Diphtheria and Tetanus Toxoids and Acellular Pertussis Adsorbed and Inactivated Poliovirus Vaccine (KINRIX® and Quadracel®); Diphtheria and Tetanus Toxoids and Acellular Pertussis Adsorbed, Inactivated Poliovirus, Haemophilus b Conjugate [Meningococcal Protein Conjugate] and Hepatitis B [Recombinant] Vaccine (VAXELIS®); Diphtheria and Tetanus Toxoids and Acellular Pertussis Adsorbed, Inactivated Poliovirus and Haemophilus b Conjugate (Tetanus Toxoid Conjugate) Vaccine (Pentacel®); Ebola Zaire Vaccine, Live (ERVEBO®); Haemophilus b Conjugate Vaccine (Meningococcal Protein Conjugate) (PedvaxHIB®); Haemophilus b Conjugate Vaccine (Tetanus Toxoid Conjugate) (ActHIB® and Hiberix®); Hepatitis A Vaccine, Inactivated (Havrix® and VAQTA®); Hepatitis A Inactivated and Hepatitis B (Recombinant) Vaccine (Twinrix®); Hepatitis B Vaccine (Recombinant) (Recombivax HB®, PREHEVBRIO® and Engerix-B®); Hepatitis B Vaccine (Recombinant), Adjuvanted (HEPLISAV-B®); Human Papillomavirus Quadrivalent (Types 6, 11, 16, 18) Vaccine, Recombinant (Gardasil®); Human Papillomavirus 9-valent Vaccine, Recombinant (Gardasil 9®); Human Papillomavirus Bivalent (Types 16, 18) Vaccine, Recombinant (Cervarix®); Influenza A (H1N1) 2009 Monovalent Vaccines (several with no trade name); Influenza Virus Vaccine, H5N1 (for National Stockpile); Influenza A (H5N1) Virus Monovalent Vaccine, Adjuvanted (No Trade Name); Influenza A (H5N1) Monovalent Vaccine, Adjuvanted (AUDENZ®); Influenza Vaccines, Adjuvanted (Fluad® and Fluad® Quadrivalent); Influenza Vaccines (Afluria® Quadrivalent and Afluria Quadrivalent Southern Hemisphere); Influenza Vaccines (Flucelvax® Quadrivalent and Flulaval® Quadrivalent); Influenza Virus Vaccines—Trivalent, Types A and B (Afluria and Afluria Southern Hemisphere, Agriflu™, FluLaval, Fluarix, Fluvirin®, Fluzone®, Fluzone® High-Dose and Fluzone® Intradermal, FlucelvaxR); Influenza Vaccine, Live, Intranasal—Trivalent, Types A and B (FluMistR); Influenza Vaccine Trivalent (Flublok®) and Quadrivalent (Flublok® Quadrivalent); Influenza Vaccine Live, Intranasal (Quadrivalent, Types A and Types B) (FluMistR Quadrivalent); Influenza Virus Vaccine (Quadrivalent, Types A and Types B) (Fluarix Quadrivalent); Influenza Virus Vaccine (Quadrivalent, Types A and Types B) (Fluzone® Quadrivalent, Fluzone® High-Dose Quadrivalent); Japanese Encephalitis Virus Vaccine, Inactivated, Adsorbed (Ixiaro); Measles, Mumps and Rubella Vaccine, Live (PRIORIX); Measles, Mumps, and Rubella Virus Vaccine, Live (M-M-RR II); Measles, Mumps, Rubella and Varicella Virus Vaccine Live (ProQuad®); Meningococcal (Groups A, C, Y, and W-135) Oligosaccharide Diphtheria CRM197 Conjugate Vaccine (MENVEO); Meningococcal (Groups A, C, Y and W-135) Polysaccharide Diphtheria Toxoid Conjugate Vaccine (Menactra®); Meningococcal Group B Vaccine (BEXSERO and TRUMENBA®); Meningococcal Polysaccharide Vaccine, Groups A, C, Y and W-135 Combined (Menomune®-A/C/Y/W-135); Meningococcal (Groups A, C, Y, W) Conjugate Vaccine (MenQuadfi®); Plague Vaccine (No trade name); Pneumococcal Vaccine, Polyvalent (Pneumovax®23); Pneumococcal 13-valent Conjugate Vaccine (Diphtheria CRM197 Protein) (Prevnar 13); Pneumococcal 15-valent Conjugate Vaccine (VAXNEUVANCE™); Pneumococcal 20-valent Conjugate Vaccine (Prevnar 20®); Poliovirus Vaccine Inactivated (Human Diploid Cell) (Poliovax); Poliovirus Vaccine Inactivated (Monkey Kidney Cell) (IPOL®); Rabies Vaccine (Imovax® and RabAvert); Rabies Vaccine Adsorbed (No Trade Name); Rotavirus Vaccine, Live, Oral (ROTARIX); Rotavirus Vaccine, Live, Oral, Pentavalent (RotaTeqR); Respiratory Syncytial Virus Vaccine (ABRYSVO™); Respiratory Syncytial Virus Vaccine, Adjuvanted (AREXVY); Smallpox and Monkeypox Vaccine, Live, Non-Replicating (JYNNEOS); Smallpox (Vaccinia) Vaccine, Live (ACAM2000®); Tetanus & Diphtheria Toxoids, Adsorbed (TDVAX™); Tetanus & Diphtheria Toxoids Adsorbed for Adult Use (TENIVAC®); Tetanus Toxoid Adsorbed (No Trade Name); Tetanus Toxoid, Reduced Diphtheria Toxoid and Acellular Pertussis Vaccine, Adsorbed (Adacel®); Tetanus Toxoid, Reduced Diphtheria Toxoid and Acellular Pertussis Vaccine, Adsorbed (Boostrix); Tick-Borne Encephalitis Vaccine (TICOVAC); Typhoid Vaccine Live Oral Ty21a (Vivotif®); Typhoid Vi Polysaccharide Vaccine (TYPHIM Vi®); Varicella Virus Vaccine Live (VarivaxR); Yellow Fever Vaccine (YF-VaxR); Zoster Vaccine, Live, (Oka/Merck) (Zostavax®); Zoster Vaccine Recombinant, Adjuvanted (SHINGRIX).

In some embodiments, the PKC activating compound is administered prior to, concurrently with, or subsequent to administration of the antigenic material. In some embodiments, the PKC activating compound is administered concurrently with the antigenic material, either separately by the same or different administration route, or together as a single composition.

In some embodiments, the PKC activating compound is administered subsequent to administration of the antigenic material. In some embodiments, the administration of the PKC activating compound is sufficiently spaced apart following the administration of the antigenic material to stimulate the immune response to the antigenic material.

In some embodiments, the PKC activating compound is administered prior to administration of the antigenic material, for example to prime the immune system for response to the subsequent exposure to the antigenic material. In some embodiments, the administration of the antigenic material following administration of the PKC activating compound is sufficiently spaced apart to stimulate the immune response to the antigenic material.

In some embodiments, the PKC activating compound is subsequently administered to the subject one or more times following an initial administration or vaccination with the PKC activating compound and antigenic material. In some embodiments, the PKC activating compound is administered once every two days, once every 4 days, once every 7 days (per week), once every 14 days (per two weeks), once every 21 days (per three weeks), or once every month, or at sufficient intervals to further stimulate the immune response against the antigenic material.

In some embodiments, the subsequent administration of the PKC activating compound is in conjunction with administration of a subsequent dose of antigenic material. In some embodiments, the subsequent administration of the PKC activating compound is done without a subsequent dose of antigenic material. In some embodiments, following initial vaccination with antigenic material and PKC activating compound, one or more doses of PKC activating compound is administered without antigenic material followed by a second vaccination with antigenic material, with or without the PKC activating compound.

### 5.3. PKC ACTIVATING COMPOUNDS AS ADJUVANTS

In some embodiments, the compounds for use as an adjuvant are protein kinase C (PKC) modulating compounds. In particular, the compounds are diterpenoid PKC modulating compounds displaying potent PKC activating activity as well as displaying enhanced solubility and pharmacokinetic profiles.

In some embodiments, the diterpenoid compounds with PKC activating activity are disclosed in U.S. Pat. Nos. 6,432,452; 8,022,103; 8,067,632; 8,431,612; 8,536,378; 8,816,122; US20090187046; US20110014699; US20120101283; US2011/0224297; WO2017083783; WO2017156350; Wender, et al., 2008, “Practical Synthesis of Prostratin, DPP, and Their Analogs, Adjuvant Leads Against Latent HIV,” Science. 320(5876):649-652; Beans et al., 2013, “Highly potent, synthetically accessible prostratin analogs induce latent HIV expression in vitro and ex vivo,” Proc Natl Acad Sci USA 110(29):11698-11703; Tsai et al., 2016, “Isolation of Phorbol Esters from Euphorbia grandicornis and Evaluation of Protein Kinase C- and Human Platelet-Activating Effects of Euphorbiaceae Diterpenes,” J Nat Prod. 79(10):2658-2666; Duran-Pena et al., 2014, Natural Product Reports 31:940-952; Shi et al., 2008, Chem. Rev. 108:4295-4327; and U.S. Pat. No. 10,183,922 (e.g., wound healing); all publications incorporated herein by reference.

In some embodiments, the diterpenoid compounds with PKC activating activity are based on ingenane or ingenol structure, such as those disclosed in U.S. Pat. Nos. 6,432,452; 8,022,103; 8,106,092; 8,431,612; 8,901,356; 9,102,687; US 20080069809; US 2010204318; US 20130324600; US 20130331446; US 20140371311; US 20150175622; WO20130182688; WO2014066967; Jorgensen et al., 2013, “14-Step Synthesis of (+)-Ingenol from (+)-3-Carene,” Science 341(6148):878-882; McKerral et al., 2014, “Development of a Concise Synthesis of (+)-Ingenol,” J. Am Chem Soc. 136 (15):5799-5810; Liang et al., 2013, Bioorg Med Chem Lett. 23:5624-5629; Grue-Sorensen et al., 2014, “Synthesis, biological evaluation and SAR of 3-benzoates of ingenol for treatment of actinic keratosis and non-melanoma skin cancer,” Bioorg Med Chem Lett. 24:54-60; Duran-Pena et al., 2014, Natural Product Reports 31:940-952; Shi et al., 2008, Chem. Rev. 108:4295-4327; and Yang et al., 2014, Fitoterapia 97:211-218; all of which are incorporated herein by reference.

In some embodiments, the present invention relates to the compounds disclosed herein. In some embodiments, the compounds are for use in the methods described herein. In some embodiments, the compound is a compound of formula (I):

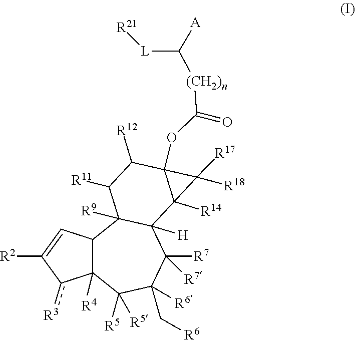

or a pharmaceutically acceptable salt, tautomer, or stereoisomer thereof;
   wherein
A is —OH, —C(O)OR′, or —NR¹³R¹³
R¹ is H or a M⁺ counterion;
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R^{5′} and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

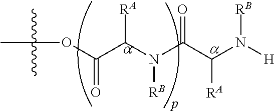

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments of the compound of formula (I), the carbon atom marked with “*”

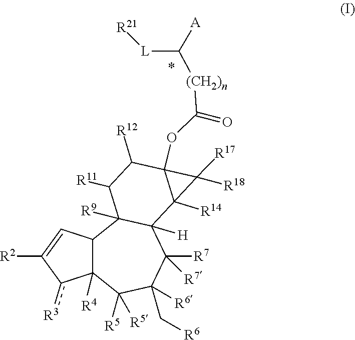

is chiral and thus can be present in S or R stereochemical configuration. In some embodiments, the stereochemical configuration is S isomer. In some embodiments, the stereochemical configuration is R isomer.

In some embodiments, A is —OH. In some embodiments, A is —C(O)OR¹, wherein R¹ is H or a M⁺ counterion. In some embodiments, A is —NR¹³R^{13′}, wherein R¹³ and R^{13′} are each independently H or C₁-C₄alkyl.

In the embodiments herein, M⁺ is a metal cation, an ammonium group, or a suitable organic cation. In some embodiments, M⁺ is a cation of an alkaline or alkaline earth metal, for example, K⁺, Na⁺, Li⁺, or Ca⁺². In some embodiments, M⁺is an ammonium ion NH₄⁺, or an organic cation derived from an amine.

In some embodiments, the compound has the structure of formula (Ia):

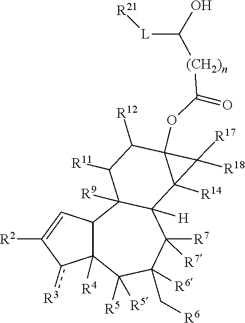

or a pharmaceutically acceptable salt, tautomer, or stereoisomer thereof;
   wherein
A is —OH;
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is

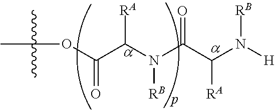

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to
form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
R^{k} is H or M⁺ counterion;
J¹ is selected from OH, CN, halo, C₁-C₄alkyl, and haloC₁-C₄alkyl; and
n is 0 or 1.

In some embodiments of the compound of formula (Ta), the carbon atom marked with “*”

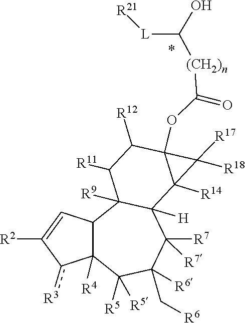

is chiral and thus can be present in S or R stereochemical configuration. In some embodiments, the stereochemical configuration is S isomer. In some embodiments, the stereochemical configuration is R isomer.

In some embodiments, the compound has the structure of formula (Ib):

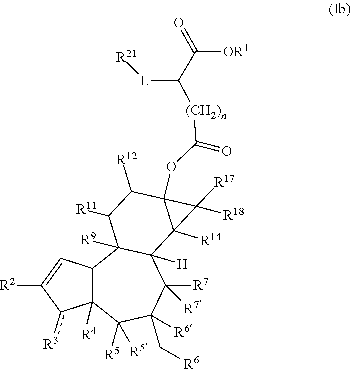

or a pharmaceutically acceptable salt, tautomer, or stereoisomer thereof;
   wherein
A is —C(O)OR′;
R¹ is H or a M⁺ counterion;
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c1} together with the N atom form a 5 to 7 membered heterocyclyl containing 1 to 3 heteroatoms selected from N, O, and S; or
R⁶ is

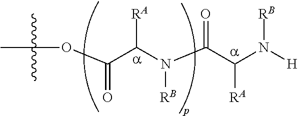

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷ is H, or R⁶′ and R⁷ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
R^{k} is H or M⁺ counterion;
J¹ is selected from OH, CN, halo, C₁-C₄alkyl, and haloC₁-C₄alkyl; and
n is 0 or 1.

In some embodiments of the compound of formula (Ib), the carbon atom marked with “*”

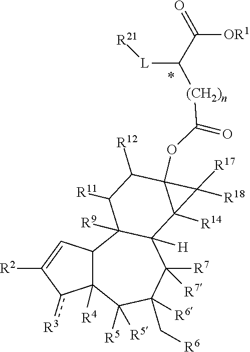

is chiral and thus can be present in S or R stereochemical configuration. In some embodiments, the stereochemical configuration is S isomer. In some embodiments, the stereochemical configuration is R isomer.

In some embodiments, the compound is a compound of formula (II):

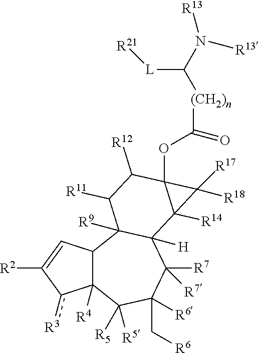

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

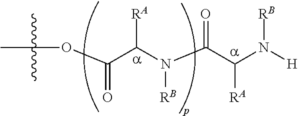

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷ is H, or R⁶′ and R⁷ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments of the compound of formula (II), the carbon atom marked with “*”

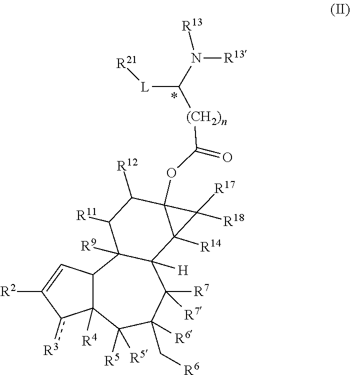

is chiral and thus compound may be present in S or R stereochemical configuration. In some embodiments, the stereochemical configuration is S isomer. In some embodiments, the stereochemical configuration is R isomer. In some embodiments, the compound of formula (II) has the structure of formula (II′):

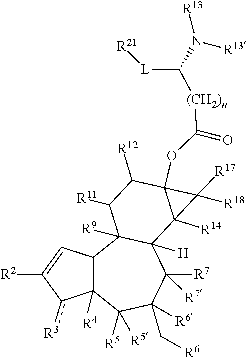

or a pharmaceutically acceptable salt, tautomer, or stereoisomer thereof, wherein R², R³, R⁴, R⁵, R⁵′, R⁶, R⁶′, R⁷, R^{7′}, R⁹, R¹¹, R¹², R¹³, R^{13′}, R¹⁴, R¹⁷, R¹⁸, L and R²¹ are as defined for formula (II).

In some embodiments, the compound of formula (II) has the structure of formula (II″):

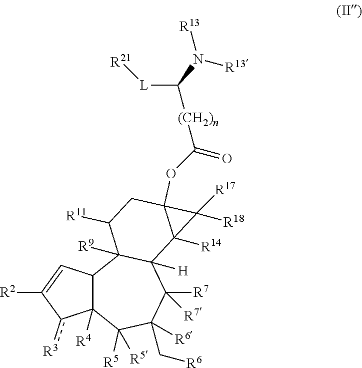

or a pharmaceutically acceptable salt, tautomer, or stereoisomer thereof, wherein R², R³, R⁴, R⁵, R⁵′, R⁶, R⁶′, R⁷, R⁷′, R⁹, R¹¹, R¹², R¹³, R^{13′}, R¹⁴, R¹⁷, R¹⁸, L and R²¹ are as defined for formula (II).

In some embodiments, the compound has the structure of formula (IIa):

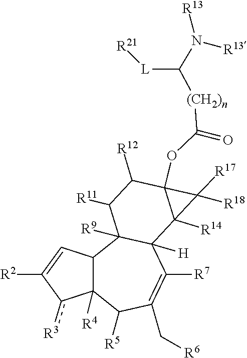

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; or
R⁶ is

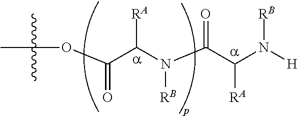

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR⁹; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments, the compound of formula (IIa) has the structure for formula (IIa′):

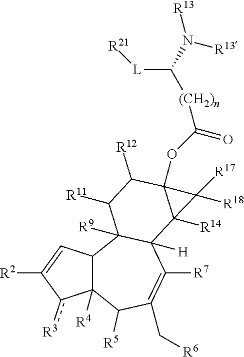

or a pharmaceutically acceptable salt, tautomer, or stereoisomer thereof, wherein R², R³, R⁴, R⁵, R⁶, R⁷, R⁹, R¹¹, R¹², R¹³, R^{13′}, R¹⁴, R¹⁷, R¹⁸, L and R²¹ are as defined for formula (II).

In some embodiments, the compound of formula (IIa) has the structure for formula (IIa″):

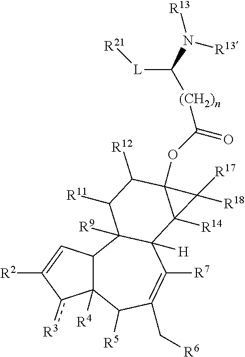

or a pharmaceutically acceptable salt, tautomer, or stereoisomer thereof, wherein R², R³′ R⁴, R⁵, R⁶, R⁷, R⁹, R¹¹, R¹², R¹³, R^{13′}, R¹⁴, R¹⁷, R¹⁸, L and R²¹ are as defined for formula (II).

In some embodiments, the compound has the structure of formula (IIb):

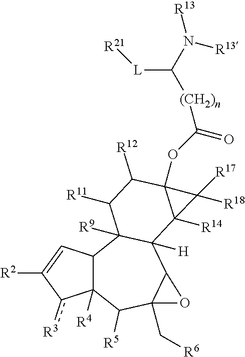

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

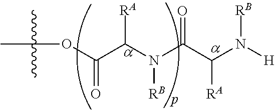

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (IIb′):

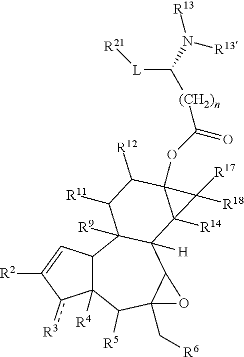

or a pharmaceutically acceptable salt, tautomer, or stereoisomer thereof, wherein R², R³, R⁴, R⁵, R⁶, R⁹, R¹¹, R¹², R¹³, R^{13′}, R¹⁴, R¹⁷, R¹⁸, L and R²¹ are as defined for formula (II).

In some embodiments, the compound has the structure of formula (IIb″):

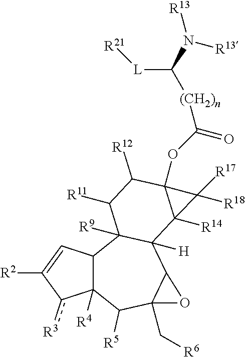

or a pharmaceutically acceptable salt, tautomer, or stereoisomer thereof, wherein R², R³′ R⁴, R⁵, R⁶, R⁹, R¹¹, R¹², R¹³, R^{13′}, R¹⁴, R¹⁷, R¹⁸, L and R²¹ are as defined for formula (II).

In some embodiments of the compound of formula (I), (Ia), (Ib), (II), (II′), (II″), (IIa), (IIa′), (IIa″), (IIb), (IIb′), and (IIb″), R³ is —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl. In some embodiments, the aryl of C₀-C₆alkylaryl is phenyl. In some embodiments, the aryl of C₀-C₆alkylaryl is optionally substituted with 1 to 3 of OH, CN, halo, C₁-C₄alkyl, and haloC₁-C₄alkyl. In some embodiments, R^{a1} is selected from:

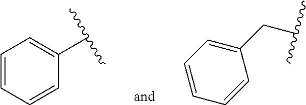 [SMILES: *c1ccccc1 ; *Cc1ccccc1]

In some embodiments, R^{a1} is selected from:

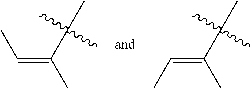

In some embodiments of the compound of formula (I), (Ia), (Ib), (II), (II′), (II″), (IIa), (IIa′), (IIa″), (IIb), (IIb′), and (IIb″),
R³ is O double bonded to the carbon atom.

In some embodiments of the compound of formula (I), (Ia), (Ib), (II), (II′), (II″), (IIa), (IIa′), (IIa″), (IIb), (IIb′), and (IIb″), one or more of R², R¹¹, R¹⁷, and R¹⁸ are —CH₃. In some embodiments, each of R², R¹¹, R¹⁷, and R¹⁸ is —CH₃.

In some embodiments of the compound of formula (I), (Ia), (Ib), (II), (II′), (II″), (IIa), (IIa′), (IIa″), (IIb), (IIb′), and (IIb″), R⁴ and R⁵ are each independently H or —OH.

In some embodiments of the compound of formula (I), (Ia), (Ib), (II), (II′), (II″), (IIa), (IIa′), (IIa″), (IIb), (IIb′), and (IIb″),
R², R¹¹, R¹⁷, and R¹⁸ are —CH₃;
R³ is O double bonded to the carbon atom; and
R⁴ and R⁵ are each independently H or —OH.

In some embodiments, the compound has the structure of formula (IIc):

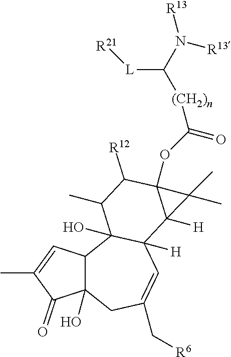

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

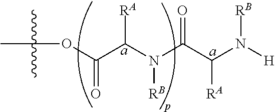

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
   each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
   R^{k} is H or M⁺ counterion;
   J¹ is selected from OH, CN, halo, C₁-C₄alkyl, and haloC₁-C₄alkyl; and
   n is 0 or 1.

In some embodiments, the compound has the structure of formula (IIc′):

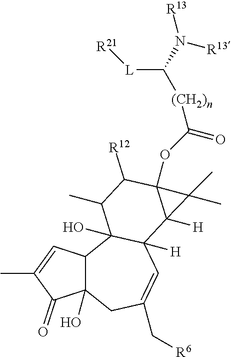

or a pharmaceutically acceptable salt, tautomer, or stereoisomer thereof, wherein R⁶, R¹², R¹³, R^{13′}, L and R²¹ are as defined for formula (IIc).

In some embodiments, the compound has the structure of formula (IIc″):

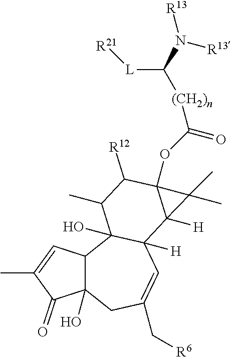

or a pharmaceutically acceptable salt, tautomer, or stereoisomer thereof, wherein R⁶, R¹², R¹³, R^{13′}, L and R²¹ are as defined for formula (IIc).

In some embodiments, the compound has the structure of formula (IId):

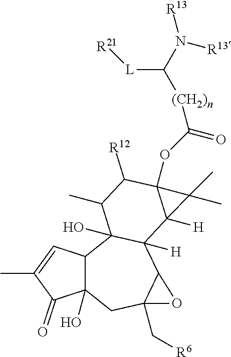

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

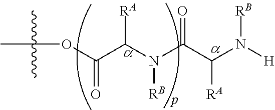

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (IId′):

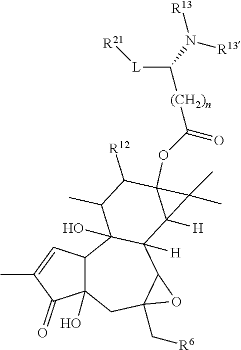

or a pharmaceutically acceptable salt, tautomer, or stereoisomer thereof, wherein R⁶, R¹², R¹³, R^{13′}, L and R²¹ are as defined for formula (IId).

In some embodiments, the compound has the structure of formula (IId″):

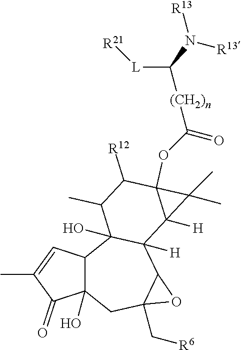

or a pharmaceutically acceptable salt, tautomer, or stereoisomer thereof, wherein R⁶, R¹², R¹³, R^{13′}, L and R²¹ are as defined for formula (IId).

In some embodiments of the compound of formula (I), (Ia), (Ib), (II), (II′), (II″), (IIa), (IIa′), (IIa″), (IIb), (IIb′), (IIb″), (IIc), (IIc″), (IId), (IId′) and (IId″), R¹² is —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl. In some embodiments, R^{f} is selected from

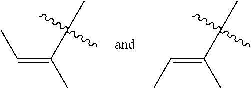

In some embodiments, R^{f} is selected from:

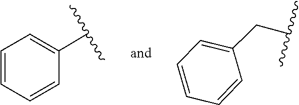 [SMILES: *c1ccccc1 ; *Cc1ccccc1]

In some embodiments, the compound has the structure of formula (III):

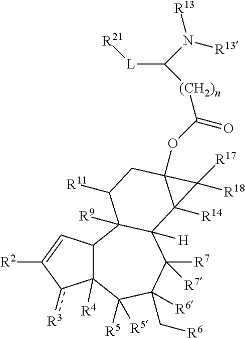

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

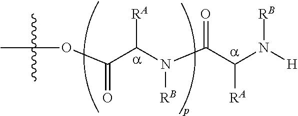

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷ is H, or R⁶′ and R⁷ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (III′):

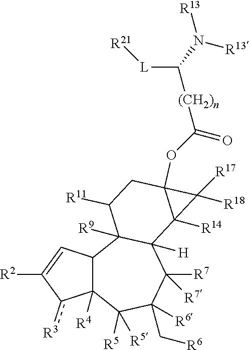

or a pharmaceutically acceptable salt, tautomer, or stereoisomer thereof, wherein R², R³, R⁴, R⁵, R⁵′, R⁶′, R⁶, R⁶′, R^{7′}, R⁷, R⁹, R¹¹, R¹³, R^{13′}, R¹⁴, R¹⁷, R¹⁸, L and R²¹ are as defined for formula (III).

In some embodiments, the compound has the structure of formula (III″):

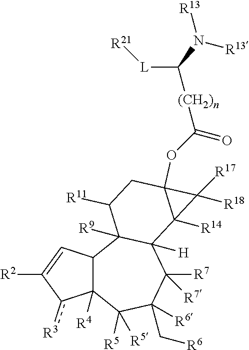

or a pharmaceutically acceptable salt, tautomer, or stereoisomer thereof, wherein R², R³′ R⁴, R⁵, R⁵′, R⁶′, R⁶, R⁶′, R^{7′}, R⁷, R⁹, R¹¹, R¹³, R^{13′}, R¹⁴, R¹⁷, R¹⁸, L and R²¹ are as defined for formula (III).

In some embodiments, the compound has the structure of formula (IIIa):

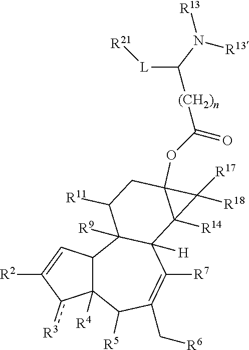

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; or
R⁶ is

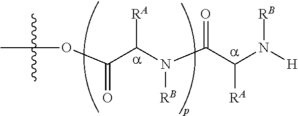

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹³ and R^{13′} are each independently H or C₁-C₄alkyl;
R¹⁴ is H or OR⁹; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (IIIa′):

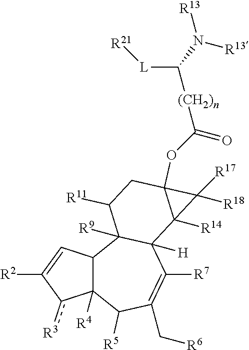

or a pharmaceutically acceptable salt, tautomer, or stereoisomer thereof, wherein R², R³, R⁴, R⁵, R⁶, R⁷, R⁹, R, R¹², R¹³, R^{13′}, R¹⁴, R¹⁷, R¹⁸, L and R²¹ are as defined for formula (III).

In some embodiments, the compound has the structure of formula (IIIa″):

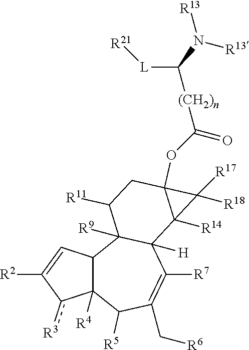

or a pharmaceutically acceptable salt, tautomer, or stereoisomer thereof, wherein R², R³′ R⁴, R⁵, R⁶, R, R⁹, R¹¹, R¹², R¹³, R^{13′}, R¹⁴, R¹⁷, R¹⁸, L and R²¹ are as defined for formula (III).

In some embodiments, the compound has the structure of formula (IIIb):

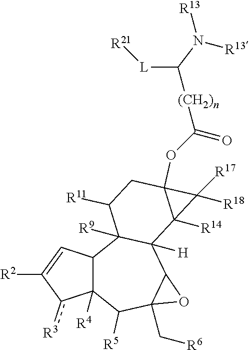

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c} is H, C₁-C₆alkyl, or two R¹ together with the N atom form a 5 to 7 membered heterocyclyl containing 1 to 3 heteroatoms selected from N, O, and S; or
R⁶ is

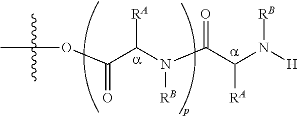

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁹ is OR^{e}, wherein R^{e} is H, C₁-C₆alkyl, or aryl;
R¹¹ is C₁-C₄alkyl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (IIIb′):

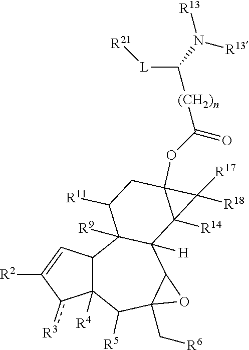

or a pharmaceutically acceptable salt, tautomer, or stereoisomer thereof, wherein R², R³, R⁴, R⁵, R⁶, R⁹, R¹¹, R¹³, R^{13′}, R¹⁴, R¹⁷, R¹⁸, L and R²¹ are as defined for formula (III).

In some embodiments, the compound has the structure of formula (IIIb″):

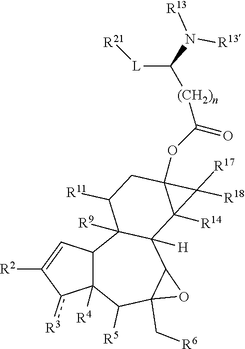

or a pharmaceutically acceptable salt, tautomer, or stereoisomer thereof, wherein R², R³′ R⁴, R⁵, R⁶, R⁹, R¹¹, R¹³, R^{13′}, R¹⁴, R¹⁷, R¹⁸, L and R²¹ are as defined for formula (III).

In some embodiments of the compound of formula (III), (III′), (III″), (IIIa), (IIIa′), (IIIa″), (IIIb), (IIIb′) and (IIIb″), R³ is —OR^{a}; wherein R^{a} is H or —C(O)R^{a1}, wherein Ral is C₁-C₆alkyl, C₂-C₆alkenyl, C₀-C₆alkylaryl, or C₀-C₆alkylheteroaryl. In some embodiments, the aryl of C₀-C₆alkylaryl is phenyl. In some embodiments, the aryl of C₀-C₆alkylaryl is optionally substituted with 1 to 3 of OH, CN, halo, C₁-C₄alkyl, and haloC₁-C₄alkyl. In some embodiments, R^{a1} is selected from:

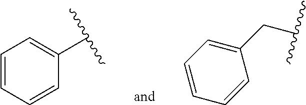 [SMILES: *c1ccccc1 ; *Cc1ccccc1]

In some embodiments, R^{a1} is selected from:

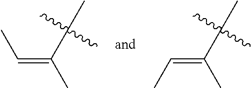

In some embodiments of the compound of formula (III), (III′), (III″), (IIIa), (IIIa′), (IIIa″), (IIIb), (IIIb′) and (IIIb″),
R³ is O double bonded to the carbon atom.

In some embodiments of the compound of formula (III), (III′), (III″), (IIIa), (IIIa′), (IIIa″), (IIIb), (IIIb′) and (IIIb″), one or more of R², R¹¹, R¹⁷, and R¹⁸ are —CH₃. In some embodiments, each of R², R¹¹, R¹⁷, and R¹⁸ is —CH₃.

In some embodiments of the compound of formula (III), (III′), (IIIa), (IIIa′), (IIIb), and (IIIb′), R⁴ and R⁵ are each independently H or —OH.

In some embodiments of the compound of formula (III), (III′), (IIIa), (IIIa′), (IIIb), and (IIIb′),
R², R¹¹, R¹⁷, and R¹⁸ are —CH₃;
R³ is O double bonded to the carbon atom; and
R⁴ and R⁵ are each independently H or —OH.

In some embodiments, the compound has the structure of formula (IIIc):

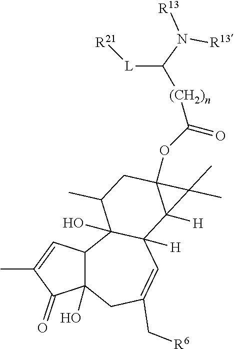

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

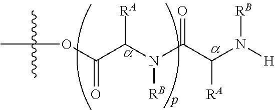

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (IIIc′):

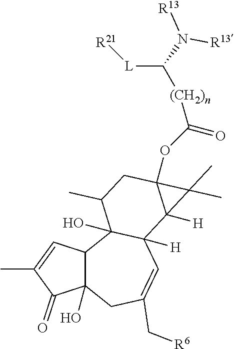

or a pharmaceutically acceptable salt, tautomer, or stereoisomer thereof, wherein R⁶, R¹³, R^{13′}, L and R²¹ are as defined for formula (IIIc).

In some embodiments, the compound has the structure of formula (IIIc″):

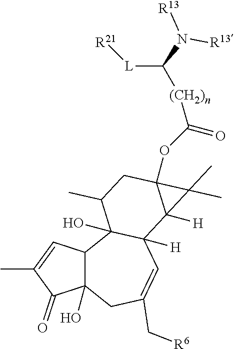

or a pharmaceutically acceptable salt, tautomer, or stereoisomer thereof, wherein R⁶, R¹³, R^{13′}, L and R²¹ are as defined for formula (IIIc).

In some embodiments, the compound has the structure of formula (IIId):

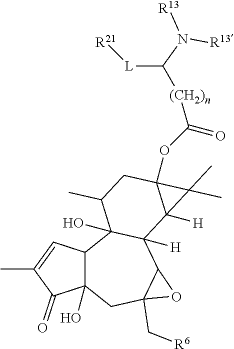

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

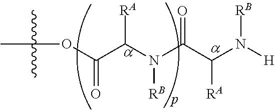

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
R^{k} is H or M⁺ counterion;
J¹ is selected from OH, CN, halo, C₁-C₄alkyl, and haloC₁-C₄alkyl; and
n is 0 or 1.

In some embodiments, the compound has the structure of formula (IIId′):

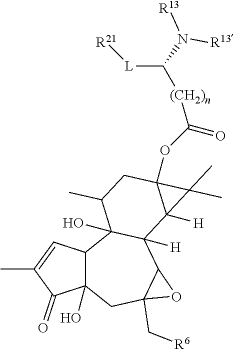

or a pharmaceutically acceptable salt, tautomer, or stereoisomer thereof, wherein R⁶, R¹³, R^{13′}, L and R²¹ are as defined for formula (IIId).

In some embodiments, the compound has the structure of formula (IIId″):

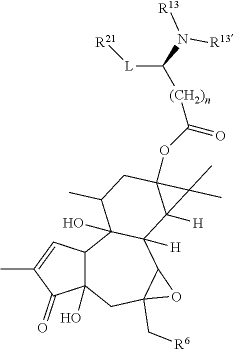

or a pharmaceutically acceptable salt, tautomer, or stereoisomer thereof, wherein R⁶, R¹³, R^{13′}, L and R²¹ are as defined for formula (IIId).

In some embodiments for the compound of any of the foregoing embodiments, n is 0.

In some embodiments for the compound of any of the foregoing embodiments, each of R¹³ and R¹³′ is H.

In some embodiments, the compound has the structure of formula (IIIe):

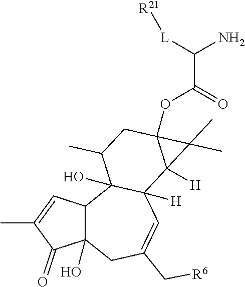

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

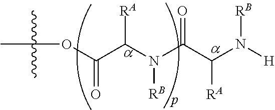

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion; and
J¹ is selected from OH, CN, halo, C₁-C₄alkyl, and haloC₁-C₄alkyl.

In some embodiments, the compound has the structure of formula (IIIe′):

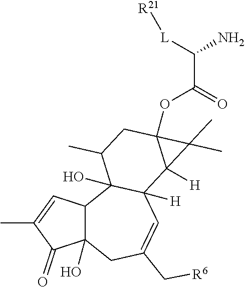

or a pharmaceutically acceptable salt, tautomer, or stereoisomer thereof, wherein R⁶, L and R²¹ are as defined for formula (IIIe).

In some embodiments, the compound has the structure of formula (IIIe″):

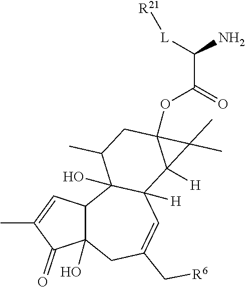

or a pharmaceutically acceptable salt, tautomer, or stereoisomer thereof, wherein R⁶, L and R²¹ are as defined for formula (IIIe).

In some embodiments, the compound has the structure of formula (IIIf):

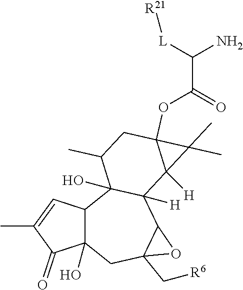

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

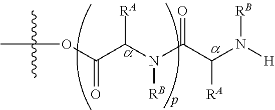

wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion; and
J¹ is selected from OH, CN, halo, C₁-C₄alkyl, and haloC₁-C₄alkyl.

In some embodiments, the compound has the structure of formula (IIIf′):

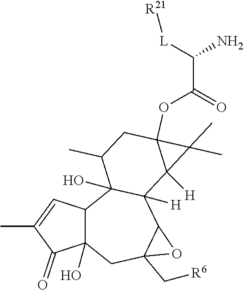

or a pharmaceutically acceptable salt, tautomer, or stereoisomer thereof,
wherein R⁶, L and R²¹ are as defined for formula (IIIf).

In some embodiments, the compound has the structure of formula (IIIf′):

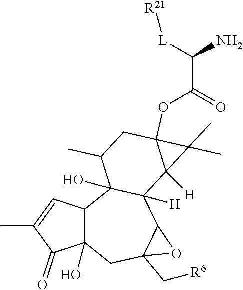

or a pharmaceutically acceptable salt, tautomer, or stereoisomer thereof, wherein R⁶, L and R²¹ are as defined for formula (IIIf).

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is C₃-C₇cycloalkyl, wherein the C₃-C₇cycloalkyl is optionally substituted with 1 to 3 of J¹, wherein J¹ is selected from OH, CN, halo, C₁-C₄alkyl, and haloC₁-C₄alkyl. In some of the foregoing embodiments, the C₃-C₇cycloalkyl is selected from the group consisting of cyclobutyl, cyclopentyl, cyclohexyl, and cycloheptyl.

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is a heterocyclyl, wherein the heterocyclyl is optionally substituted with 1 to 3 of J¹, wherein J¹ is selected from OH, CN, halo, C₁-C₄alkyl, and haloC₁-C₄alkyl. In some embodiments, the heterocyclyl is selected from oxiranyl, oxetanyl, azetidynyl, oxazolyl, thiazolidinyl, thiazolyl, morpholinyl, pyrrolidinonyl, pyrrolidinyl, piperidinyl, piperazinyl, 2,3-dihydrofuranyl, dihydropyranyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and azapanyl, wherein the heterocyclyl is optionally substituted with 1 to 3 of OH, CN, halo, C₁-C₄alkyl, and haloC₁-C₄alkyl.

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is aryl, wherein the aryl is optionally substituted with 1 to 3 of J¹, wherein J¹ is selected from OH, CN, halo, C₁-C₄alkyl, and haloC₁-C₄alkyl. In some of the foregoing embodiments, R²¹ is a phenyl or naphthyl, wherein the phenyl or naphthyl is optionally substituted with 1 to 3 of OH, CN, halo, C₁-C₄alkyl, and haloC₁-C₄alkyl.

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is heteroaryl, wherein the heteroaryl is optionally substituted with 1 to 3 of J¹, wherein J¹ is selected from OH, CN, halo, C₁-C₄alkyl, and haloC₁-C₄alkyl. In some of the foregoing embodiments, the heteroaryl is selected from the group consisting of pyrrolyl, pyrazolyl, imidazolyl, pyrazinyl, oxazolyl, isoxazolyl, thiazolyl, furyl, thienyl, pyridyl, pyrimidyl, benzoxazolyl, benzothiazolyl, purinyl, benzimidazolyl, indolyl, isoquinolyl, quinoxalinyl, and quinolyl, wherein the heteroaryl is optionally substituted with 1 to 3 of OH, CN, halo, C₁-C₄alkyl, and haloC₁-C₄alkyl.

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is adamantyl, wherein the adamantyl is optionally substituted with J¹, wherein J¹ is selected from OH, halo, C₁-C₄alkyl, and haloC₁-C₄alkyl.

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is spiroC₅-C₁₂cycloalkyl, wherein the spiroC₅-C₁₂ cycloalkyl has 0-2 carbon atoms replaced with 0-2 heteroatoms selected from N, O and S, and is optionally substituted with 1 to 3 of J¹, wherein J¹ is selected from OH, CN, halo, C₁-C₄alkyl, and haloC₁-C₄alkyl, or when an N atom is present, is optionally substituted with an N-protecting group.

In some embodiments of the compounds of (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is 5 to 12 membered bridged bicyclyl, wherein the bridged bicyclyl has 0-2 carbon atoms replaced with 0-2 heteroatoms selected from N, O and S, and is optionally substituted with 1 to 3 of J¹, wherein J¹ is selected from OH, CN, halo, C₁-C₄alkyl, and haloC₁-C₄alkyl, or when an N atom is present, is optionally substituted with an N-protecting group.

In some embodiments of the compounds of formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R²¹ is selected from the following:

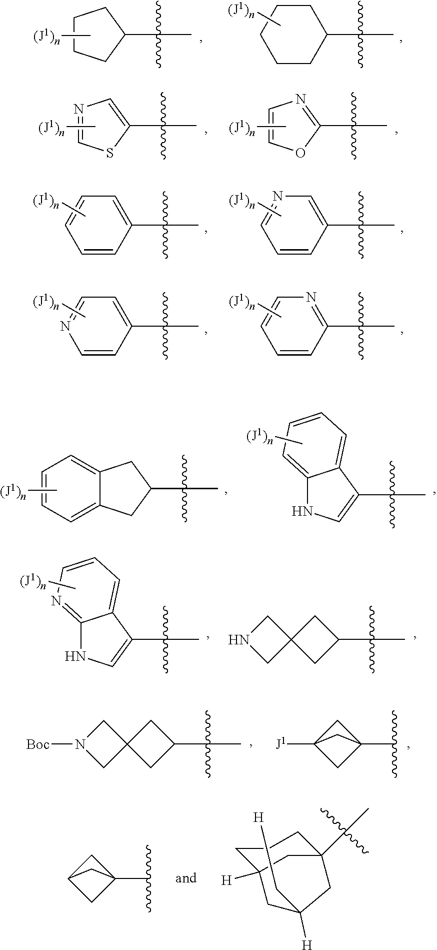

wherein J¹ is OH, CN, halo, C₁-C₄alkyl, and haloC₁-C₄alkyl, and n is 0-3. In some embodiments, n is 0. In some embodiments, n is 1. In some embodiments, n is 2. In some embodiments, haloC₁-C₄alkyl is —CH₂F, —CHF₂, or —CF₃.

In some embodiments of the compounds of formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R⁶′ is OH. In some such embodiments, R⁶ is OH. In other such embodiments, R⁶ is OH, R⁵ is H, and R⁵′ is H. In other such embodiments, R⁶ is OH, R⁵ is H, R⁵′ is H, and R⁴ is OH. In other such embodiments, the compound is not:

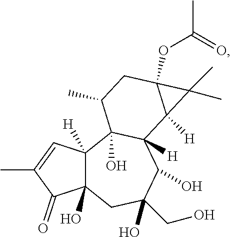

or a pharmaceutically acceptable salt thereof.

In some embodiments of the compounds of formula (I), (Ia), (Ib), (II), (II′), (II″), (IIa), (IIa′), (IIa″), (IIb), (IIb′), (IIb″), (III), (III′), (II″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R⁵ is OH. In some such embodiments, R⁵′ is OH. In other such embodiments, R⁶′ is OH. In other such embodiments, R⁶′ is H.

In some embodiments of the compounds of formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R⁶ is OP(O)(OR^{b}′)₂, wherein each R^{b}′ is independently H, C₁-C₆alkyl, C₂-C₆alkenyl, C₀-C₆alkylaryl, or C₀-C₆alkylheteroaryl. In some embodiments, R⁶ is —OP(O)(OR^{b}′)₂, wherein each R^{b}′ is independently C₁-C₆alkyl. In some embodiments, R⁶ is OP(O)(OR^{b}′)₂, wherein each R^{b}′ is independently C₂-C₆alkenyl, C₀-C₆alkylaryl. In some embodiments, R⁶ is OP(O)(OR^{b}′)₂, wherein each R^{b}′ is independently C₀-C₆alkylheteroaryl. In some such foregoing embodiments, R⁶′ is H. In other such embodiments, R⁶′ is OH.

In some embodiments of the compounds of formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), R⁶ is —OC(O)R^{c}, wherein R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c1} together with the N atom form a 5 to 7 membered heterocyclyl containing 1 to 3 heteroatoms selected from N, O, and S; and R^{k} is H or a M⁺ counterion. In some such foregoing embodiments, R⁶′ is H. In other such embodiments, R⁶′ is OH.

In some embodiments, for any of the compounds herein, L is C₃-C₁₂alkylene. In some embodiments, for any of the compounds herein, L is C₃-C₆alkylene. In some embodiments, for any of the compounds herein, L is C₁-C₆alkylene.

In some embodiments, for any of the compounds herein, L is C₃-C₁₂alkenylene. In some embodiments, for any of the compounds herein, L is C₃-C₆alkenylene. In some embodiments, for any of the compounds herein, L is C₁-C₆alkenylene.

In some embodiments, L is C₁-C₁₂alkylene, or C₂-C₁₂alkenylene, wherein the C₁-C₁₂alkylene or C₂-C₁₂alkenylene is optionally substituted with C₁-C₄alkyl; and R²¹ is H.

In some embodiments of the compounds of formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf), -L-R²¹ is a C₂-C₆alkenyl selected from:

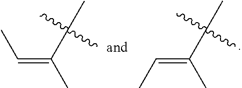

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf′), wherein R⁶ is

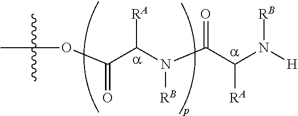

each occurrence of R^{A} is independently hydrogen (glycine), methyl (alanine), propan-2-yl (valine), propan-1-y1 (norvaline), 2-methylpropan-1-y1 (leucine), 1-methylpropan-1-y1 (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each occurrence of R^{B} is independently H, or R^{B} together with the adjacent R_{A} and the N atom form a prolyl side chain:

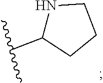 [SMILES: *C1CCCN1]

and
p is 0, 1 or 2.

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), wherein R⁶ is

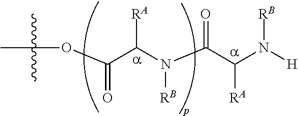

each occurrence of R^{A} is independently methyl (alanine), propan-2-y1 (valine), 2-methylpropan-1-y1 (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-y1 (arginine), benzyl (phenylalanine), or 4-aminobutan-1-y1 (lysine);
R^{B} is H; and
p 0, 1, or 2.

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf″), wherein R⁶ is

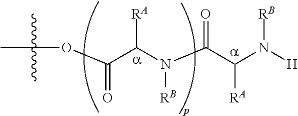

each occurrence of R^{A} is independently propan-2-y1 (valine), 2-methylpropan-1-y1 (leucine), carboxymethyl (aspartic acid), benzyl (phenylalanine), or 4-aminobutan-1-y1 (lysine);
each R^{B} is H; and
p is 0, 1, or 2.

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf′), p is 0.

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf′), and (IIIf′), p is 1.

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf), and (IIIf′), wherein R⁶ is

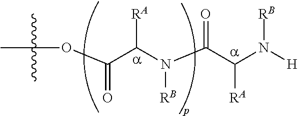

and p is 1;
first of R^{A} is propan-2-y1 (valine) and second of R^{A} is propan-2-y1 (valine); and each of R^{B} is H (i.e., dipeptide Val-Val); or
first of R^{A} is 2-methylpropan-1-y1 (leucine), and second of R^{A} is 2-methylpropan-1-y1 (leucine); and each of R^{B} is H (i.e., dipeptide Leu-Leu); or
first of R^{A} is methyl (alanine) and second of R^{A} is methyl (alanine); and each of R^{B} is H (i.e., dipeptide Ala-Ala); or
first of R^{A} is 4-aminobutan-1-y1 (lysine); second of R^{A} is 4-aminobutan-1-y1 (lysine); and each of R^{B} is H (i.e., dipeptide Lys-Lys); or
first of R^{A} is hydrogen; second of R^{A} is 4-aminobutan-1-y1, and each of R^{B} is H (i.e., dipeptide Gly-Lys).

In some embodiments for any of the foregoing compounds, e.g., formula (I), (Ia), (Ib), (II), (II′), (II″), (IIa), (IIa′), (IIa″), (IIb), (IIb′), (IIb″), (III), (III′), (III″), (IIIa), (IIIa′), (IIIa″), (IIIb), (IIIb′), (IIIb″), (IIIc), (IIIc′), (IIIc″), (IIId), (IIId′), (IIId″), (IIIe), (IIIe′), (IIIe″), (IIIf), (IIIf), and (IIIf′), wherein R⁶ is

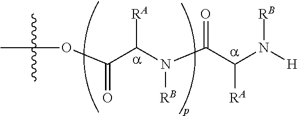

each of the α-carbon of the amino acid other than glycine is in the L or D configuration. In some embodiments, the α-carbon of the amino acid other than glycine is in the L configuration

In some embodiments, the compound is selected from the group consisting of the compounds or a pharmaceutical salt thereof in Table 1:

TABLE 1

| | |
|---|---|
| 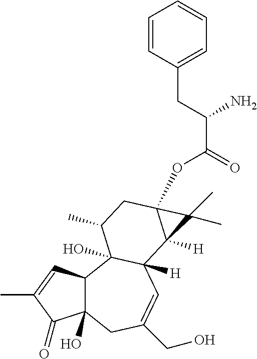 [SMILES: CC1=C[C@H]2[C@@]3(O)[C@H](C)C[C@]4(OC(=O)[C@@H](N)Cc5ccccc5)[C@H]([C@@H]3C=C(CO)C[C@]2(O)C1=O)C4(C)C] | K101-C1303 |
| 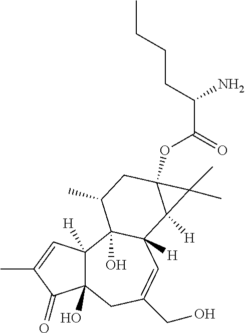 | K101-C1317 |
| 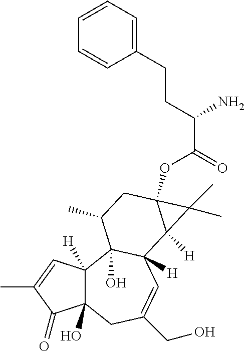 | K101-C1318 |
| 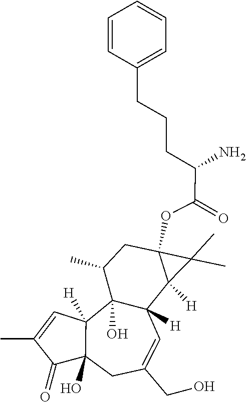 | K101-C1319 |
| 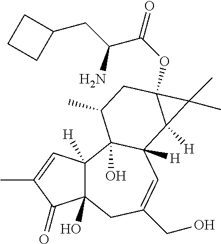 | K101-C1325 |
| 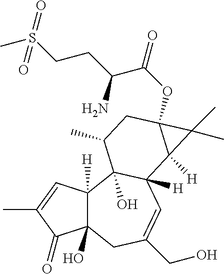 | K101-C1326 |
| 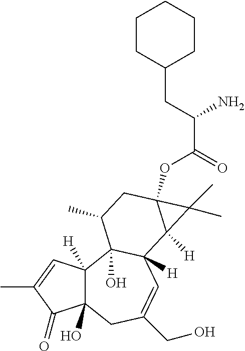 | K101-C1327 |
| 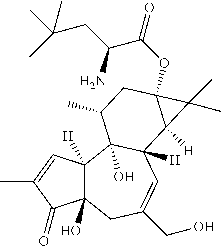 | K101-C1328 |
| 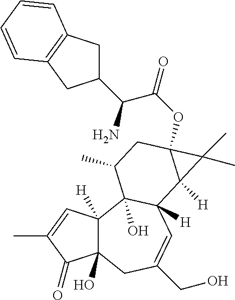 | K101-C1329 |
| 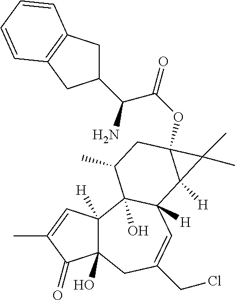 | K101-C1329-C1 |
| 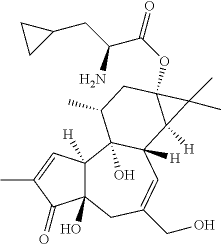 | K101-C1330 |
| 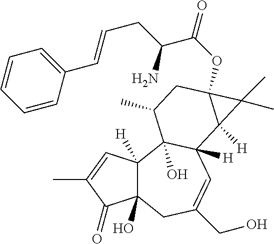 | K101-C1331 |
| 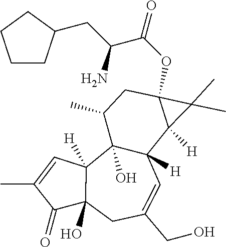 | K101-C1332 |
| 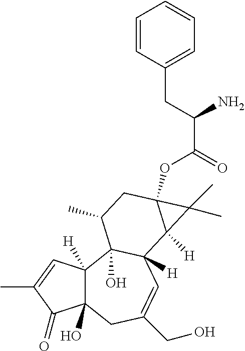 | K101-C1336 |
| 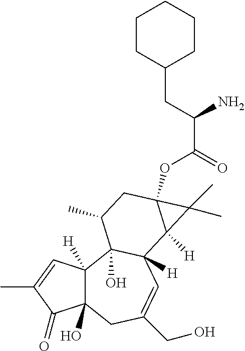 | K101-C1337 |
| 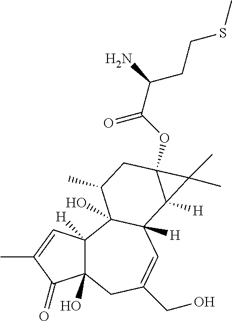 | K101-C1338 |
| 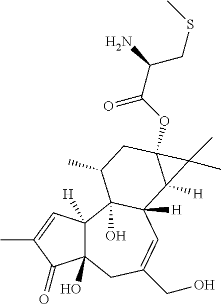 | K101-C1339 |
| 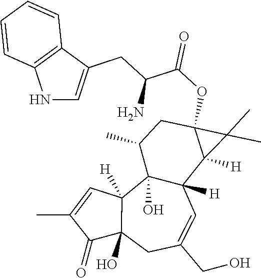 | K101-C1340 |
| 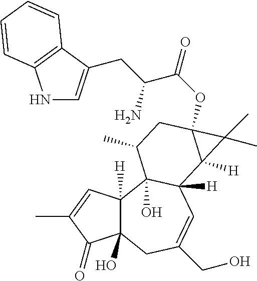 | K101-C1341 |
| 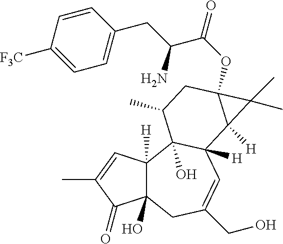 | K101-C1342 |
| 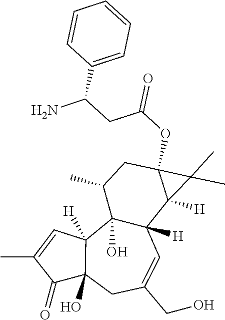 | K101-C1343 |
| 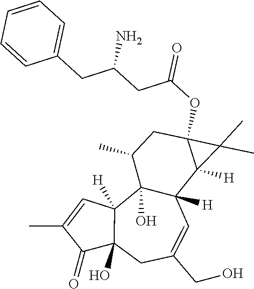 | K101-C1344 |
| 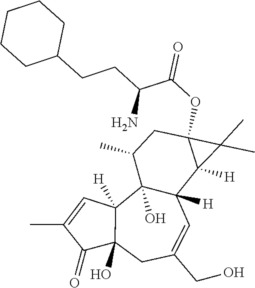 | K101-C1345 |
| 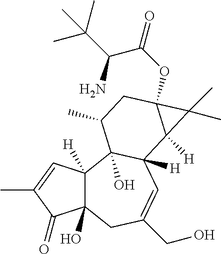 | K101-C1346 |
| 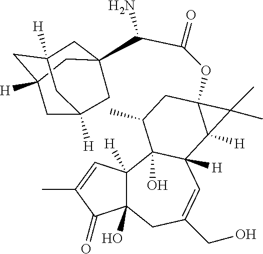 | K101-C1347 |
| 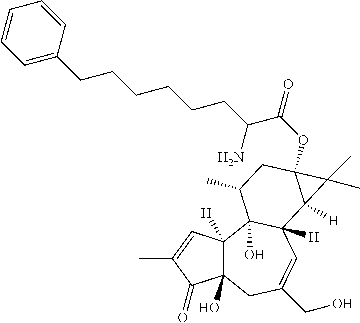 | K101-C1348 |
| 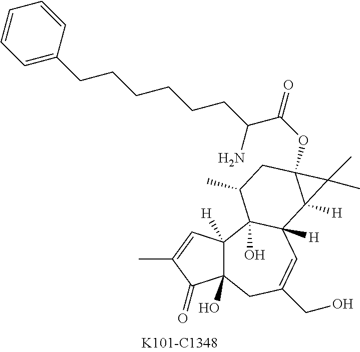 | K101-C134802 |
| 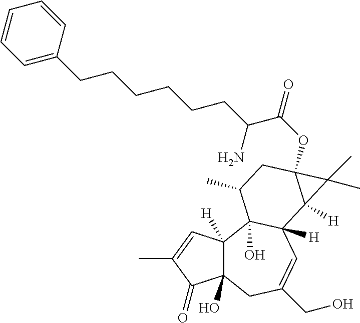 | K101-C134801 |
| 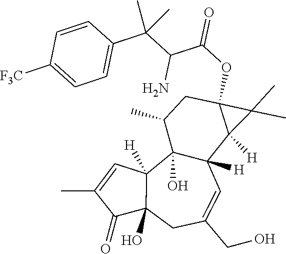 | K101-C1349 |
| 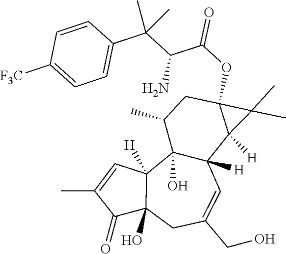 | K101-C134901 |
| 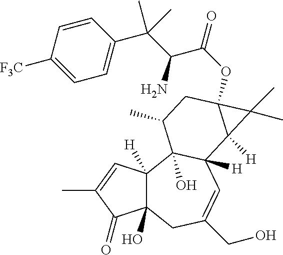 | K101-C134902 |
| 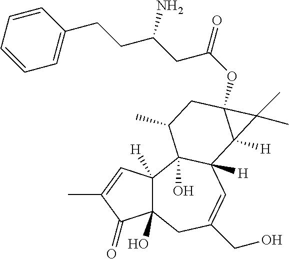 | K101-C1350 |
| 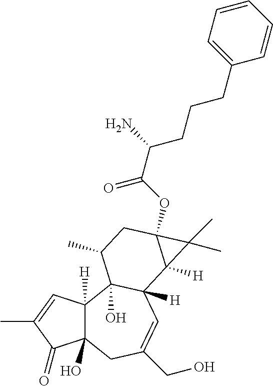 | K101-C1351 |
| 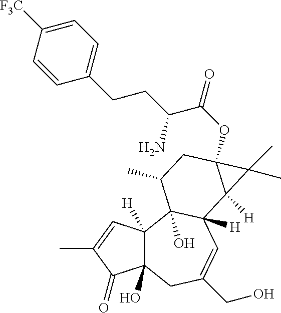 | K101-C135601 |
| 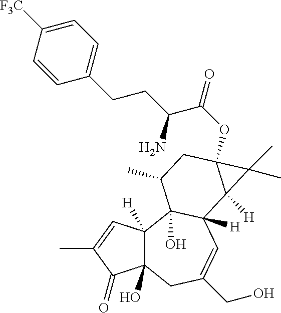 | K101-C135602 |
| 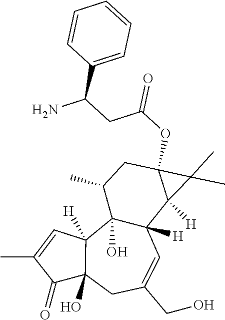 | K101-C1357 |
| 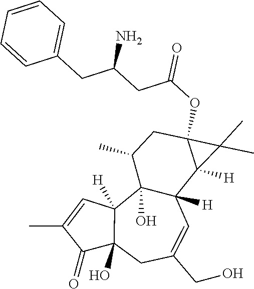 | K101-C1358 |
| 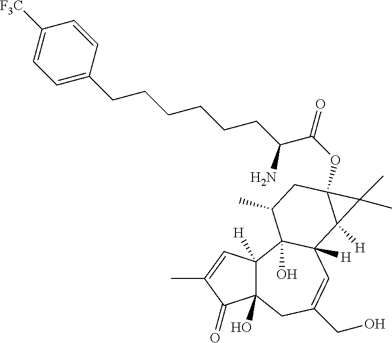 | K101-C1359 |
| 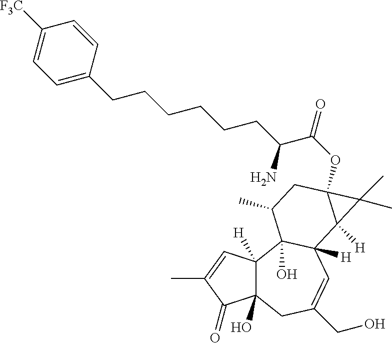 | K101-C135901 |
| 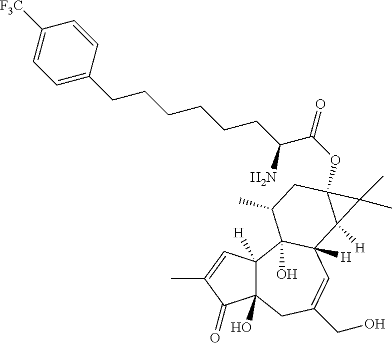 | K101-C135902 |
| 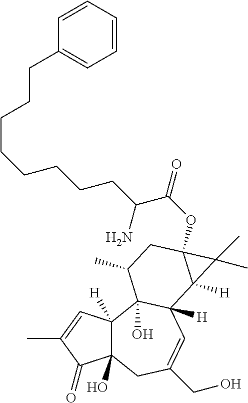 | K101-C1361 |
| 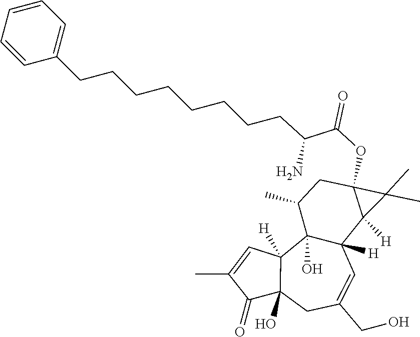 | K101-C136101 |
| 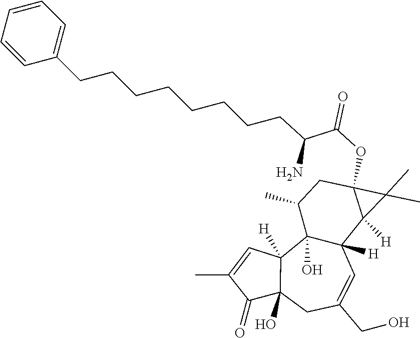 | K101-C136102 |
| 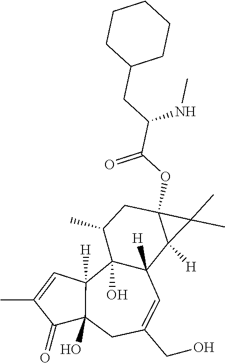 | K101-C1364 |
| 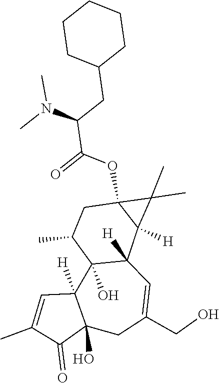 | K101-C1365 |
| 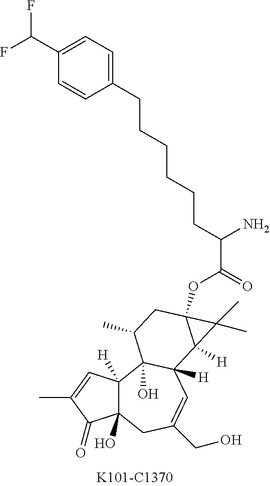 | K101-C1370 |
| 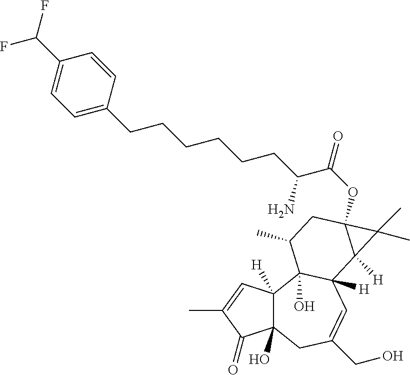 | K101-C137001 |
| 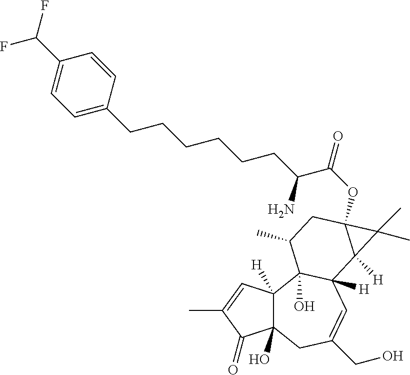 | K101-C137002 |
| 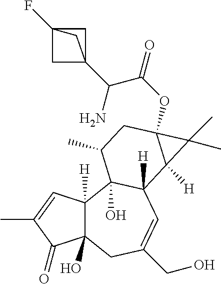 | K101-C1373 |
| 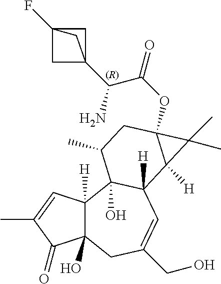 | K101-C137301 |
| 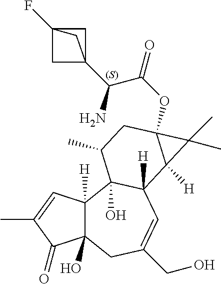 | K101-C137302 |
| 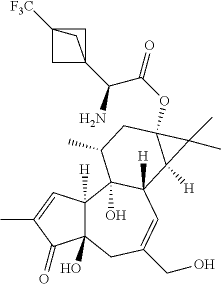 | K101-C1375 |
| 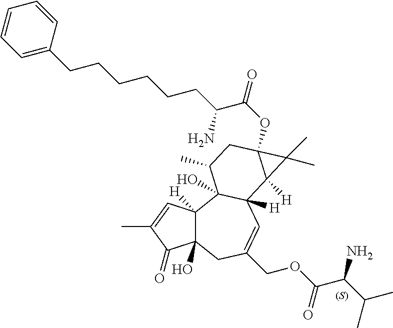 | K101-C134801C2003 |

In some embodiments, for each of the compounds of Table 1, the substituent on the C₂₀ carbon atom, for example —OH, -halo, or amino acid progroup, is replaced with —OP(O)(OR^{b}′)₂, wherein each R^{b}′ is independently H, C₁-C₆alkyl, C₂-C₆alkenyl, C₀-C₆alkylaryl, or C₀-C₆alkylheteroaryl. In some embodiments, each R^{b}′ is H. In some embodiments, each R^{b}′ is independently C₁-C₆alkyl. In some embodiments, each R^{b}′ is independently C₂-C₆alkenyl. In some embodiments, each R^{b}′ is independently C₀-C₆alkylaryl. In some embodiments, each R^{b}′ is independently C₀-C₆alkylheteroaryl.

In some embodiments, for each of the compounds of Table 1, the substituent on the C₂₀ carbon atom, for example —OH, -halo, or amino acid progroup, is replaced with —OC(O)R^{c}, wherein R^{c} is —C₁-C₆alkyl, —C₁-C₆alkyl-(NR^{c1})₂ or —C₁-C₆alkylC(O)OR^{k}; R¹ is H, C₁-C₆alkyl, or two R^{c1} together with the N atom form a 5 to 7 membered heterocyclyl containing 1 to 3 heteroatoms selected from N, O, and S; and R^{k} is H or a M⁺ counterion.

In some embodiments, for each of the compounds of Table 1, the substituent on the C20 carbon atom, where appropriate, for example —OH or -halo, is replaced with

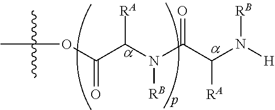

wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

In some embodiments of the amino acid moiety on the C20 carbon,
each occurrence of R^{A} is independently hydrogen (glycine), methyl (alanine), propan-2-yl (valine), propan-1-y1 (norvaline), 2-methylpropan-1-y1 (leucine), 1-methylpropan-1-y1 (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each occurrence of R^{B} is independently H, or R^{B} together with the adjacent R_{A} and the N atom form a prolyl side chain:

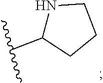 [SMILES: *C1CCCN1]

and
p is 0, 1 or 2.

In some embodiments, each occurrence of R^{A} is independently methyl (alanine), propan-2-y1 (valine), 2-methylpropan-1-y1 (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-y1 (arginine), benzyl (phenylalanine), or 4-aminobutan-1-y1 (lysine);
R^{B} is H; and
p 0, 1, or 2.

In some embodiments, each occurrence of R^{A} is independently propan-2-y1 (valine), 2-methylpropan-1-y1 (leucine), carboxymethyl (aspartic acid), benzyl (phenylalanine), or 4-aminobutan-1-y1 (lysine);
each R^{B} is H; and
p is 0, 1, or 2.

In some embodiments, p is 0.

In some embodiments, p is 1.

In some embodiments, p is 1;
first of R^{A} is propan-2-y1 (valine) and second of R^{A} is propan-2-y1 (valine); and each of R^{B} is H (i.e., dipeptide Val-Val); or
first of R^{A} is 2-methylpropan-1-y1 (leucine), and second of R^{A} is 2-methylpropan-1-y1 (leucine); and each of R^{B} is H (i.e., dipeptide Leu-Leu); or
first of R^{A} is methyl (alanine) and second of R^{A} is methyl (alanine); and each of R^{B} is H (i.e., dipeptide Ala-Ala); or
first of R^{A} is 4-aminobutan-1-y1 (lysine); second of R^{A} is 4-aminobutan-1-y1 (lysine); and each of R^{B} is H (i.e., dipeptide Lys-Lys); or
first of R^{A} is hydrogen; second of R^{A} is 4-aminobutan-1-y1, and each of R^{B} is H (i.e., dipeptide Gly-Lys).

In some embodiments, each of the α-carbon of the amino acid other than glycine is in the L or D configuration. In some embodiments, each of the α-carbon of the amino acid other than glycine is in the L configuration.

In another aspect, the present disclosure provides a compound of formula (IV):

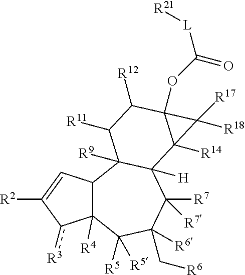

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

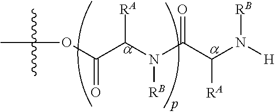

wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR⁹; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, excluded from the compounds of formula (IV) are compounds in which:
-L-R²¹ is

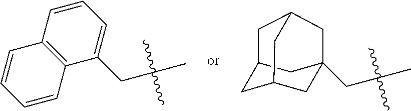 [SMILES: *Cc1cccc2ccccc12 ; *CC12CC3CC(CC(C3)C1)C2]

R² and R¹¹ are CH₃;
R³ is ═O;
R⁴ is OH;
R⁵, R^{5′}, R⁷ and R¹⁴ are H;
R⁶′ and R⁷′ together form a bond;
R⁹ is OH;
R¹² is H;
R¹⁷ and R¹⁸ are —CH₃;
R^{A} is propan-2-yl (valine);
R^{B} is H; and
p is 0.

In some embodiments, the compound has the structure of formula (IVa):

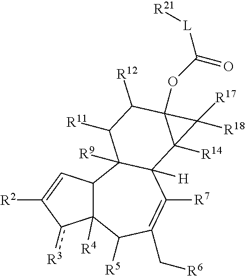

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c} together with the N atom form a 5 to 7 membered heterocyclyl containing 1 to 3 heteroatoms selected from N, O, and S; or
R⁶ is

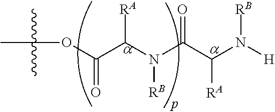

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, excluded from the compounds of formula (IVa) are compounds in which:
-L-R²¹ is

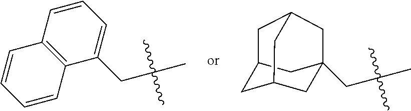 [SMILES: *Cc1cccc2ccccc12 ; *CC12CC3CC(CC(C3)C1)C2]

R² and R¹¹ are CH₃;
R³ is ═O;
R⁴ is OH;
R⁵, R⁷ and R¹⁴ are H;
R⁹ is OH;
R¹² is H;
R¹⁷ and R¹⁸ are —CH₃;
R^{A} is propan-2-yl (valine);
R^{B} is H; and
p is 0.

In some embodiments, the compound has the structure of formula (IVb)

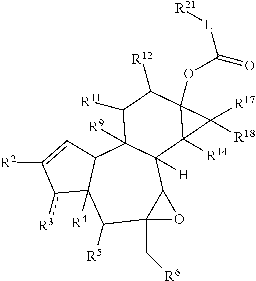

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

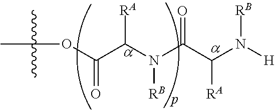

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, the compound has the structure of formula (IVc):

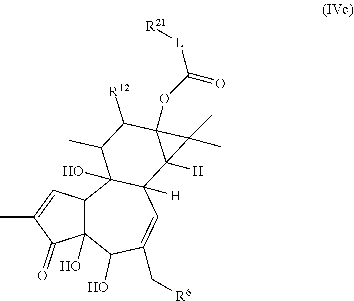

or a pharmaceutically acceptable salt, tautomer, or stereoisomer thereof;
   wherein
R⁶ is OH, halo, or —OC(O)R^{c}, wherein R^{c} is —C₁-C₆alkyl, —C₁-C₆alkyl-(NR^{c1})₂ or —C₁-C₆alkylC(O)OR^{k}, R^{c1} is H, C₁-C₆alkyl, or two R^{c} together with the N atom form a 5 to 7 membered heterocyclyl containing 1 to 3 heteroatoms selected from N, O, and S; or
R⁶ is

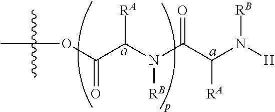

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
J¹ is selected from OH, CN, halo, C₁-C₄alkyl, and haloC₁-C₄alkyl; and
R^{k} is H or M⁺ counterion.

In some embodiments, the compound has the structure of formula (IVd):

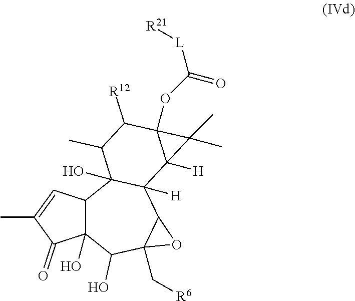

or a pharmaceutically acceptable salt, tautomer, or stereoisomer thereof;
   wherein
R⁶ is OH, halo, or —OC(O)R^{c}, wherein R^{c} is —C₁-C₆alkyl, —C₁-C₆alkyl-(NR^{c1})₂ or —C₁-C₆alkylC(O)OR^{k}, R^{c1} is H, C₁-C₆alkyl, or two R^{c} together with the N atom form a 5 to 7 membered heterocyclyl containing 1 to 3 heteroatoms selected from N, O, and S; or
R⁶ is
wherein,

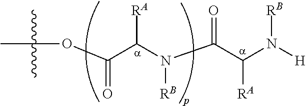

each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, the compound has the structure of formula IVe or IVf, or an amino acid prodrug thereof:

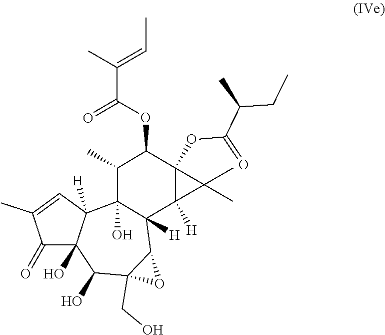

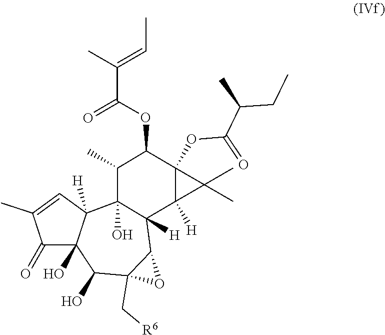

wherein for the structural formula IVf, R⁶ is

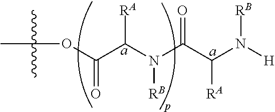

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2.

In some embodiments of the compound of formula (IV), (IVa), (IVb), (IVc), and (IVd), the C₂-C₆alkenyl of R^{a1} is independently selected from:

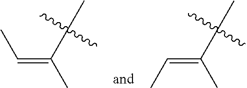

In some embodiments, R^{a1} is independently selected from:

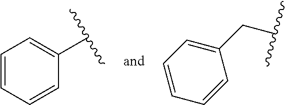 [SMILES: *c1ccccc1 ; *Cc1ccccc1]

In some embodiments, R³ is —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl. In some embodiments, the aryl of C₀-C₆alkylaryl is phenyl. In some embodiments, the aryl of C₀-C₆alkylaryl is optionally substituted with 1 to 3 of OH, CN, halo, C₁-C₄alkyl, and haloC₁-C₄alkyl.

In some embodiments of the compound of formula (IV), (IVa), and (IVb),
R³ is O double bonded to the carbon atom.

In some embodiments of the compound of formula (IV), (IVa), and (IVb), the C₂-C₁₂alkenyl of R^{f} is independently selected from:

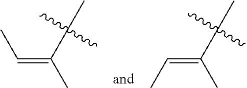

In some embodiments, R^{f} is independently selected from:

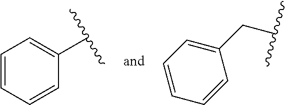 [SMILES: *c1ccccc1 ; *Cc1ccccc1]

In some embodiments of the compound of formula (IV), (IVa), and (IVb), one or more of R², R¹¹, R¹⁷, and R¹⁸ are —CH₃. In some embodiments, each of R², R¹¹, R¹⁷, and R¹⁸ is —CH₃.

In some embodiments of the compound of formula (IV), (IVa), and (IVb), R⁴ and R⁵ are each independently H or —OH.

In some embodiments of the compound of formula (IV), (IVa), and (IVb),
R², R¹¹, R¹⁷, and R¹⁸ are —CH₃;
R³ is O double bonded to the carbon atom; and
R⁴ and R⁵ are each independently H or —OH.

In some embodiments, the compound has the structure of formula (V):

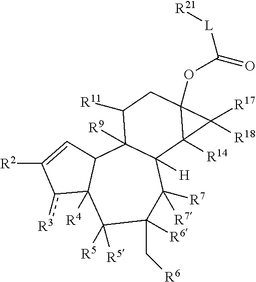

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is

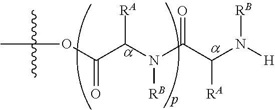

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, excluded from the compounds of formula (V) are compounds in which:
-L-R²¹ is

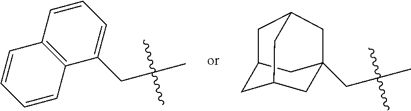 [SMILES: *Cc1cccc2ccccc12 ; *CC12CC3CC(CC(C3)C1)C2]

R² and R¹¹ are CH₃;
R³ is ═O;
R⁴ is OH;
R⁵, R^{5′}, R⁷ and R¹⁴ are H;
R⁶′ and R⁷′ together form a bond;
R⁹ is OH;
R¹⁷ and R¹⁸ are —CH₃;
R^{A} is propan-2-yl (valine);
R^{B} is H; and
p is 0.

In some embodiments, the compound has the structure of formula (Va):

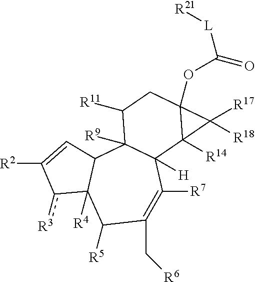

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR′1) or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is

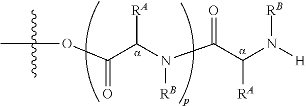

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, excluded from the compounds of formula (V) are compounds in which:
-L-R²¹ is

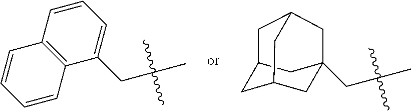 [SMILES: *Cc1cccc2ccccc12 ; *CC12CC3CC(CC(C3)C1)C2]

R² and R¹¹ are CH₃;
R³ is ═O;
R⁴ is OH;
R⁵, R⁷ and R¹⁴ are H;
R⁹ is OH;
R¹⁷ and R¹⁸ are —CH₃;
R^{A} is propan-2-yl (valine);
R^{B} is H; and
p is 0.

In some embodiments, the compound has the structure of formula (Vb):

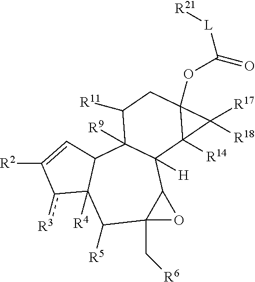

or a pharmaceutically acceptable salt, tautomer, or stereoisomer thereof;
   wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl; R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c1} together with the N atom form a 5 to 7 membered heterocyclyl containing 1 to 3 heteroatoms selected from N, O, and S; or
R⁶ is

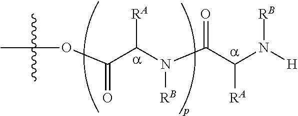

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
R⁹ is OR^{e}, wherein R^{e} is H, C₁-C₆alkyl, or aryl;
R¹¹ is C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments of the compound of formula (V), (Va), and (Vb), R³ is —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl. In some embodiments, the aryl of C₀-C₆alkylaryl is phenyl. In some embodiments, the aryl of C₀-C₆alkylaryl is optionally substituted with 1 to 3 of OH, CN, halo, C₁-C₄alkyl, and haloC₁-C₄alkyl. In some embodiments, R^{a1} is selected from:

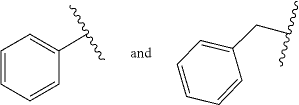 [SMILES: *c1ccccc1 ; *Cc1ccccc1]

In some embodiments, R^{a1} is selected from:

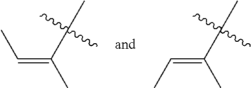

In some embodiments of the compound of formula (V), (Va), and (Vb),
R³ is O double bonded to the carbon atom.

In some embodiments of the compound of formula (V), (Va), and (Vb), one or more of R², R¹¹, R¹⁷, and R¹⁸ are —CH₃. In some embodiments, each of R², R¹¹, R¹⁷, and R¹⁸ is —CH₃.

In some embodiments of the compound of formula (V), (Va), and (Vb), R⁴ and R⁵ are each independently H or —OH.

In some embodiments of the compound of formula (V), (Va), and (Vb),
R², R¹¹, R¹⁷, and R¹⁸ are —CH₃;
R³ is O double bonded to the carbon atom; and
R⁴ and R⁵ are each independently H or —OH.

In some embodiments, the compound has the structure of formula (Vc):

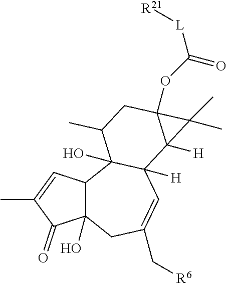

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c} is H, C₁-C₆alkyl, or two R¹ together with the N atom form a 5 to 7 membered heterocyclyl containing 1 to 3 heteroatoms selected from N, O, and S; or
R⁶ is

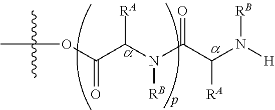

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments, excluded from the compounds of formula (Vc) are compounds in which:
-L-R²¹ is

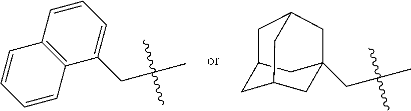 [SMILES: *Cc1cccc2ccccc12 ; *CC12CC3CC(CC(C3)C1)C2]

R^{A} is propan-2-yl (valine);
R^{B} is H; and
p is 0.

In some embodiments, specifically excluded from the compounds of formula (IV), (IVa), (V), (Va) and (Vc) are compounds of the following structure:

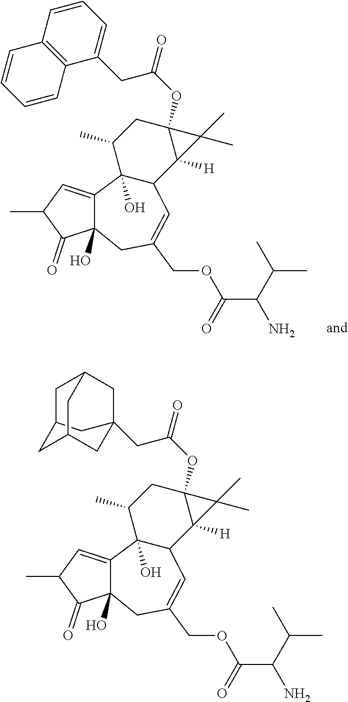

In some embodiments, the compound has the structure of formula (Vd):

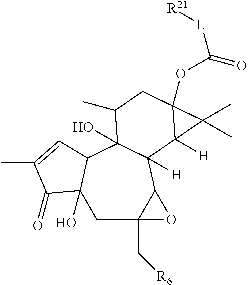

or a pharmaceutically acceptable salt, tautomer, or stereoisomer thereof;
   wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is

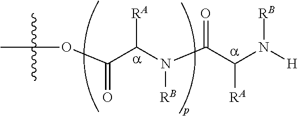

wherein,
   each occurrence of R^{A} is independently selected from a side chain of a natural or non-natural amino acid, wherein each occurrence of R^{A} is same or different;
   each occurrence of R^{B} is independently H, or R^{B} together with R^{A} and the N atom to which it is attached form a heterocyclic ring of a natural or non-natural amino acid, wherein each occurrence of R^{B} is same or different; and
   p is 0, 1, or 2;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

In some embodiments of the compounds of (IV), (IVa), (IVb), (V), (Va), (Vb), (Vc) and (Vd), R²¹ is C₃-C₇cycloalkyl, wherein the C₃-C₇cycloalkyl is optionally substituted with 1 to 3 of J¹. In some of the foregoing embodiments, the C₃-C₇cycloalkyl is selected from the group consisting of cyclobutyl, cyclopentyl, cyclohexyl, and cycloheptyl.

In some embodiments of the compounds of (IV), (IVa), (IVb), (V), (Va), (Vb), (Vc) and (Vd), R²¹ is a heterocyclyl, wherein the heterocyclyl is optionally substituted with 1 to 3 of J¹. In some embodiments, the heterocycloalkyl is selected from oxiranyl, oxetanyl, azetidynyl, oxazolyl, thiazolidinyl, thiazolyl, morpholinyl, pyrrolidinonyl, pyrrolidinyl, piperidinyl, piperazinyl, 2,3-dihydrofuranyl, dihydropyranyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and azapanyl.

In some embodiments of the compounds of (IV), (IVa), (IVb), (V), (Va), (Vb), (Vc), and (Vd), R²¹ is aryl, wherein the aryl is optionally substituted with 1 to 3 of J¹. In some of the foregoing embodiments, R²¹ is a phenyl, wherein the phenyl is optionally substituted with 1 to 3 of OH, CN, halo, C₁-C₄alkyl, and haloC₁-C₄alkyl.

In some embodiments of the compounds of (IV), (IVa), (IVb), (V), (Va), (Vb), (Vc), and (Vd), R²¹ is heteroaryl, wherein the heteroaryl is optionally substituted with 1 to 3 of J¹. In some of the foregoing embodiments, the heteroaryl is selected from the group consisting of pyrrolyl, pyrazolyl, imidazolyl, pyrazinyl, oxazolyl, isoxazolyl, thiazolyl, furyl, thienyl, pyridyl, pyrimidyl, benzoxazolyl, benzothiazolyl, purinyl, benzimidazolyl, indolyl, isoquinolyl, quinoxalinyl, and quinolyl, wherein the heteroaryl is optionally substituted with 1 to 3 of OH, CN, halo, C₁-C₄alkyl, and haloC₁-C₄alkyl.

In some embodiments of the compounds of (IV), (IVa), (IVb), (V), (Va), (Vb), (Vc), and (Vd), R²¹ is adamantyl, wherein the adamantyl is optionally substituted with OH, halo, or C₁-C₄alkyl.

In some embodiments of the compounds of (IV), (IVa), (IVb), (V), (Va), (Vb), (Vc), and (Vd), R²¹ is spiroC₅-C₁₂ cycloalkyl, wherein the spiroC₅-C₁₂ cycloalkyl has 0-2 carbon atoms replaced with 0-2 heteroatoms selected from N, O and S, and is optionally substituted with 1 to 3 of OH, CN, halo, C₁-C₄alkyl, and haloC₁-C₄alkyl, or when an N atom is present an N-protecting group.

In some embodiments of the compounds of (IV), (IVa), (IVb), (V), (Va), (Vb), (Vc) and (Vd), R²¹ is 5 to 12 membered bridged bicyclyl, wherein the bridged bicyclyl has 0-2 carbon atoms replaced with 0-2 heteroatoms selected from N, O and S, and is optionally substituted with 1 to 3 of OH, CN, halo, C₁-C₄alkyl, and haloC₁-C₄alkyl, or when an N atom is present an N-protecting group.

In some embodiments of the compounds of formula (IV), (IVa), (IVb), (V), (Va), (Vb), (Vc) and (Vd), R²¹ is selected from the following:

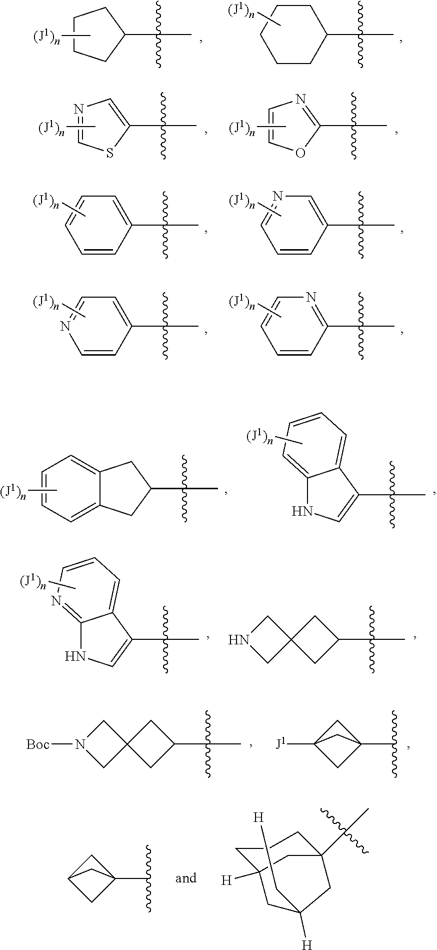

wherein J¹ is OH, CN, halo, C₁-C₄alkyl, and haloC₁-C₄alkyl, and n is 0-3. In some embodiments, n is 0. In some embodiments, n is 1. In some embodiments, n is 2. In some embodiments, haloC₁-C₄alkyl is —CH₂F, —CHF₂, or —CF₃.

In some embodiments, for any of the compounds herein, L is C₃-C₁₂alkylene. In some embodiments, for any of the compounds herein, L is C₃-C₆alkylene. In some embodiments, for any of the compounds herein, L is C₁-C₆alkylene.

In some embodiments, for any of the compounds herein, L is C₃-C₁₂alkenylene. In some embodiments, for any of the compounds herein, L is C₃-C₆alkenylene. In some embodiments, for any of the compounds herein, L is C₁-C₆alkenylene.

In some embodiments of the compounds of formula (IV), (IVa), (IVb), (V), (Va), (Vb), (Vc) and (Vd), -L-R²¹ is a C₂-C₆alkenyl selected from:

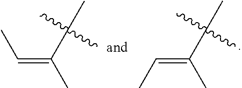

In some embodiments for any of the foregoing compounds, e.g., formula (IV), (IVa), (IVb), (V), (Va), (Vb), (Vc) and (Vd), wherein
R⁶ is

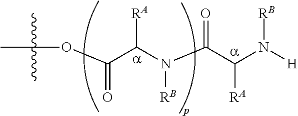

each occurrence of R^{A} is independently hydrogen (glycine), methyl (alanine), propan-2-yl (valine), propan-1-y1 (norvaline), 2-methylpropan-1-y1 (leucine), 1-methylpropan-1-y1 (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each occurrence of R^{B} is H, or R^{B} together with the adjacent R_{A} and the N atom form a prolyl side chain:

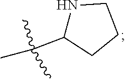 [SMILES: *C1CCCN1]

and
p is 0, 1 or 2.

In some embodiments for any of the foregoing compounds, e.g., formula (IV), (IVa), (IVb), (V), (Va), (Vb), (Vc) and (Vd), each R^{A} is independently methyl (alanine), propan-2-y1 (valine), 2-methylpropan-1-yl (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-yl (arginine), benzyl (phenylalanine), or 4-aminobutan-1-y1 (lysine);
R^{B} is H; and
p 0, 1, or 2.

In some embodiments for any of the foregoing compounds, e.g., formula (IV), (IVa), (IVb), (V), (Va), (Vb), (Vc) and (Vd), wherein
R⁶ is

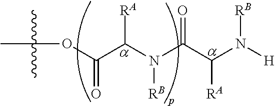

each occurrence of R^{A} is independently propan-2-y1 (valine), 2-methylpropan-1-y1 (leucine), carboxymethyl (aspartic acid), benzyl (phenylalanine), or 4-aminobutan-1-y1 (lysine);
each R^{B} is H; and
p is 0, 1, or 2.

In some embodiments for any of the foregoing compounds, e.g., formula (IV), (IVa), (IVb), (V), (Va), (Vb), (Vc) and (Vd), p is 0.

In some embodiments for any of the foregoing compounds, e.g., formula (IV), (IVa), (IVb), (V), (Va), (Vb), (Vc) and (Vd), p is 1.

In some embodiments for any of the foregoing compounds, e.g., formula (IV), (IVa), (IVb), (V), (Va), (Vb), (Vc) and (Vd), wherein
R⁶ is

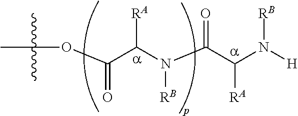

and p is 1;
first of R^{A} is propan-2-y1 (valine) and second of R^{A} is propan-2-y1 (valine); and each of R^{B} is H (i.e., dipeptide Val-Val); or
first of R^{A} is 2-methylpropan-1-y1 (leucine), and second of R^{A} is 2-methylpropan-1-y1 (leucine); and each of R^{B} is H (i.e, dipeptide Leu-Leu); or
first of R^{A} is methyl (alanine) and second of R^{A} is methyl (alanine); and each of R^{B} is H (i.e, dipeptide Ala-Ala); or
first of R^{A} is 4-aminobutan-1-y1 (lysine); second of R^{A} is 4-aminobutan-1-y1 (lysine); and each of R^{B} is H (i.e., dipeptide Lys-Lys); or
first of R^{A} is hydrogen; second of R^{A} is 4-aminobutan-1-y1, and each of R^{B} is H (i.e., dipeptide Gly-Lys).

In some embodiments for any of the foregoing compounds, e.g., formula (IV), (IVa), (IVb), (V), (Va), (Vb), (Vc) and (Vd), wherein
R⁶ is

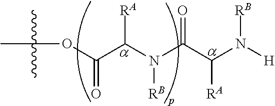

each of the α-carbon of the amino acid other than glycine is in the L or D configuration. In some embodiments, each of the α-carbon of the amino acid other than glycine is in the L configuration.

In some embodiments, the compound is selected from the group consisting of the compounds or a pharmaceutical salt thereof, in Table 2.

TABLE 2

| | |
|---|---|
| 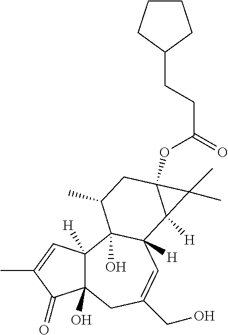 | K101-C1301 |
| 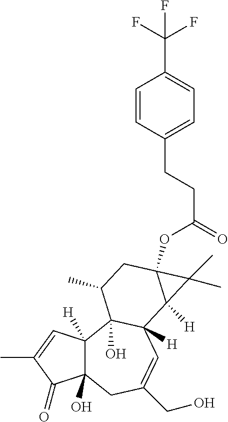 | K101-C1302 |
| 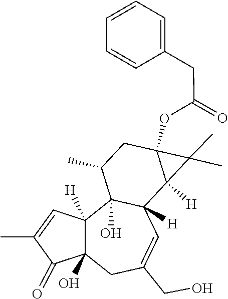 | K101-C1304 |
| 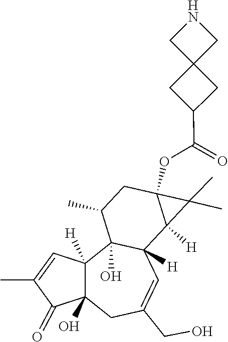 | K101-C1305 |
| 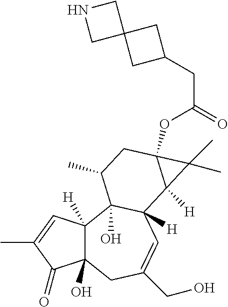 | K101-C1306 |
| 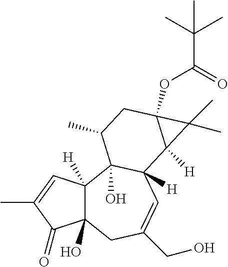 | K101-C1308 |
| 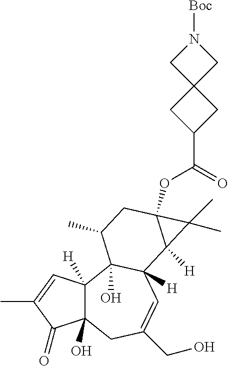 | K101-C1311 |
| 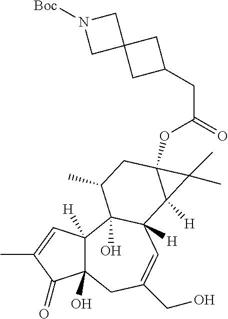 | K101-C1312 |
| 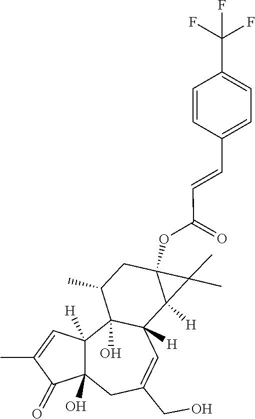 | K101-C1313 |
| 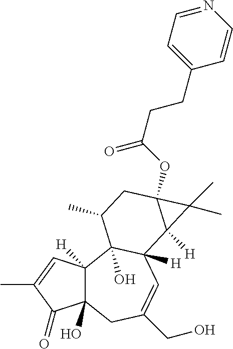 | K101-C1315 |
| 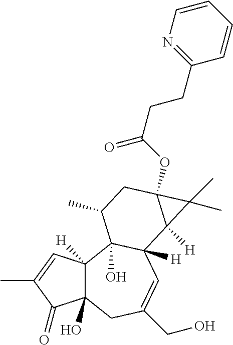 | K101-C1316 |
| 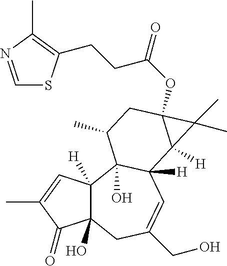 | K101-C1320 |
| 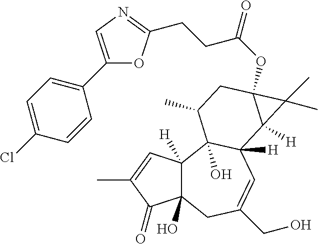 | K101-C1321 |
| 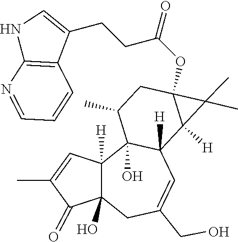 | K101-C1322 |
| 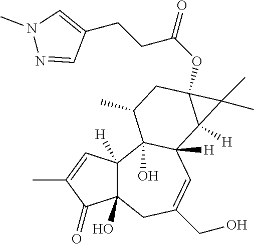 | K101-C1323 |
| 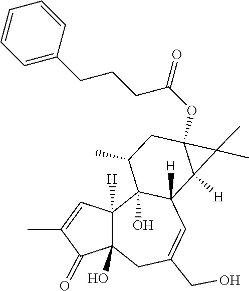 | K101-C1324 |
| 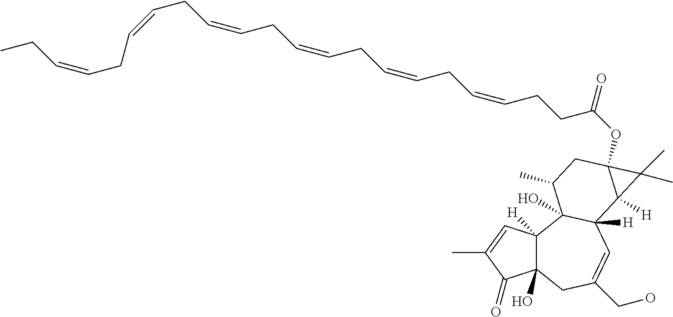 [SMILES: CC/C=C\C/C=C\C/C=C\C/C=C\C/C=C\C/C=C\CCC(=O)O[C@@]12C[C@@H](C)[C@@]3(O)[C@@H](C=C(CO)C[C@]4(O)C(=O)C(C)=C[C@@H]34)[C@@H]1C2(C)C] | K101-C1333 |
| 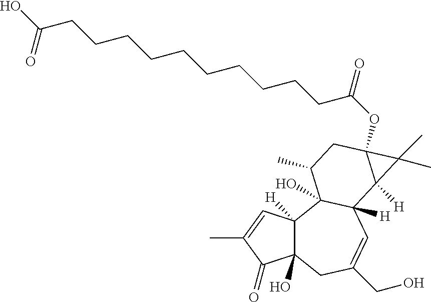 [SMILES: CC1=C[C@H]2[C@@]3(O)[C@H](C)C[C@]4(OC(=O)CCCCCCCCCCC(=O)O)[C@H]([C@@H]3C=C(CO)C[C@]2(O)C1=O)C4(C)C] | K101-C1334 |
| 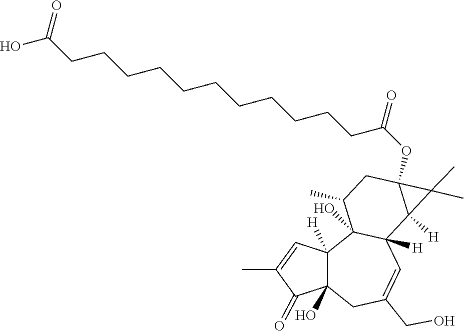 [SMILES: CC1=C[C@H]2[C@@]3(O)[C@H](C)C[C@]4(OC(=O)CCCCCCCCCCCC(=O)O)[C@H]([C@@H]3C=C(CO)C[C@]2(O)C1=O)C4(C)C] | K101-C1335 |
| 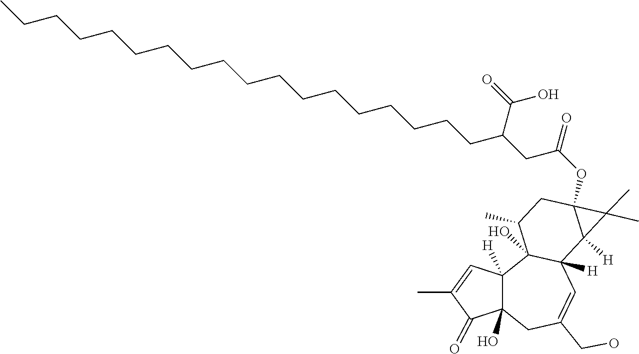 [SMILES: CCCCCCCCCCCCCCCCCCC(CC(=O)O[C@@]12C[C@@H](C)[C@@]3(O)[C@@H](C=C(CO)C[C@]4(O)C(=O)C(C)=C[C@@H]34)[C@@H]1C2(C)C)C(=O)O] | K101-C1352 |
| 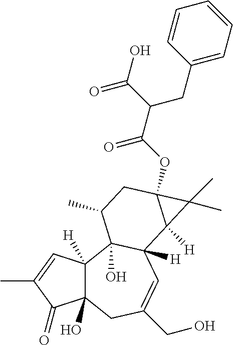 | K101-C1353 |
| 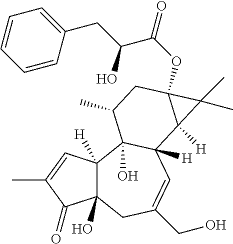 | K101-C1354 |
| 0 | |
| 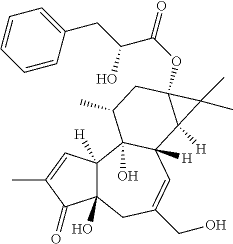 | K101-C1355 |

or a pharmaceutically acceptable salt, tautomer, or stereoisomer thereof.

In some embodiments, for each of the compounds of Table 2, the substituent on the C₂₀ carbon atom, e.g., —OH, is replaced with —OP(O)(OR^{b}′)₂, wherein each R^{b}′ is independently H, C₁-C₆alkyl, C₂-C₆alkenyl, C₀-C₆alkylaryl, or C₀-C₆alkylheteroaryl. In some embodiments, each of R^{b}′ is H. In some embodiments, each of R^{b}′ is H. In some embodiments, each R^{b}′ is independently C₁-C₆alkyl. In some embodiments, each R^{b}′ is independently C₂-C₆alkenyl. In some embodiments, each R^{b}′ is independently C₀-C₆alkylaryl. In some embodiments, each R^{b}′ is independently C₀-C₆alkylheteroaryl.

In some embodiments, for each of the compounds of Table 2, the substituent on the C₂₀ carbon atom, e.g., —OH, is replaced with —OC(O)R^{c}, wherein R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c} together with the N atom form a 5 to 7 membered heterocyclyl containing 1 to 3 heteroatoms selected from N, O, and S; and R^{k} is H or a M+counterion.

In some embodiments, for each of the compounds of Table 2, the substituent on the C20 carbon atom, e.g., —OH, is replaced with

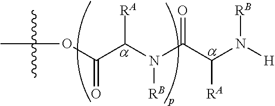

wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

In some embodiments of the amino acid moiety on the C20 carbon,
each occurrence of R^{A} is independently hydrogen (glycine), methyl (alanine), propan-2-yl (valine), propan-1-y1 (norvaline), 2-methylpropan-1-y1 (leucine), 1-methylpropan-1-y1 (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each occurrence of R^{B} is independently H, or R^{B} together with the adjacent R_{A} and the N atom form a prolyl side chain:

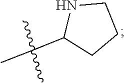 [SMILES: *C1CCCN1]

and
p is 0, 1 or 2.

In some embodiments, each occurrence of R^{A} is independently methyl (alanine), propan-2-y1 (valine), 2-methylpropan-1-y1 (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-y1 (arginine), benzyl (phenylalanine), or 4-aminobutan-1-y1 (lysine);
R^{B} is H; and
p 0, 1, or 2.

In some embodiments, each occurrence of R^{A} is independently propan-2-y1 (valine), 2-methylpropan-1-yl (leucine), carboxymethyl (aspartic acid), benzyl (phenylalanine), or 4-aminobutan-1-y1 (lysine);
each R^{B} is H; and
p is 0, 1, or 2.

In some embodiments, p is 0.

In some embodiments, p is 1.

In some embodiments,
p is 1;
first of R^{A} is propan-2-y1 (valine) and second of R^{A} is propan-2-y1 (valine); and each of R^{B} is H (dipeptide Val-Val); or
first of R^{A} is 2-methylpropan-1-y1 (leucine), and second of R^{A} is 2-methylpropan-1-y1 (leucine); and each of R^{B} is H (dipeptide Leu-Leu); or
first of R^{A} is methyl (alanine) and second of R^{A} is methyl (alanine); and each of R^{B} is H (dipeptide Ala-Ala); or
first of R^{A} is 4-aminobutan-1-y1 (lysine); second of R^{A} is 4-aminobutan-1-y1 (lysine); and each of R^{B} is H (dipeptide Lys-Lys); or
first of R^{A} is hydrogen; second of R^{A} is 4-aminobutan-1-yl, and each of R^{B} is H (dipeptide Gly-Lys).

In some embodiments, each of the α-carbon of the amino acid other than glycine is in the L or D configuration.

In some embodiments, compounds disclosed herein can be synthesized according to the general schemes shown outlined below in Scheme 1 and Scheme 2, where suitable reagents can be purchased form commercial sources or synthesized via known methods or methods adapted from the example procedures provided herein:

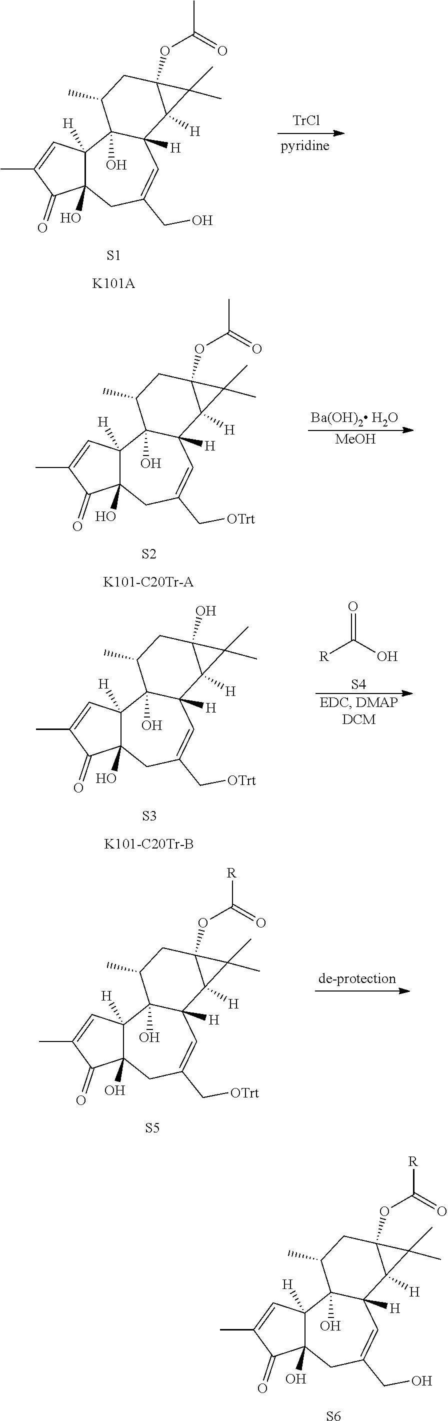

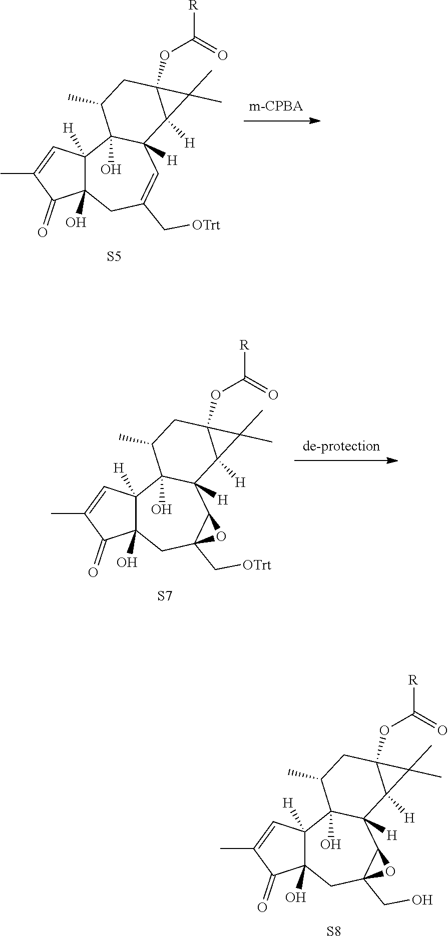

In Scheme 1, protection of S1 (K101A shown as example) with trityl chloride (or triphenylmethyl chloride) provides S2 (K101-C20Tr-A shown as example, Trt=trityl). Hydrolysis of S2 (K101-C20Tr-A shown as example) provides S3 (K101-C20Tr-B, shown as example), which can then be coupled with compound S4 under esterification conditions using 1-ethyl-3-(3-dimethylaminopropyl)carbodiimide (EDC, or EDCI) as the carboxyl activating agent and 4-dimethylaminopyridine (DMAP) as the catalyst to provide S5. Deprotection of S5 followed by further separation and purification provides S6.

In Scheme 2, S7 is prepared by epoxidation of S5 with a peroxycarboxylic acid such as meta-chloroperoxybenzoic acid (m-CPBA). Further separation and purification of S7 provides S8.

Appropriate starting materials and reagents for use in Scheme 1 and Scheme 2 can be purchased or prepared by methods known to one of skill in the art.

In some embodiments of the methods of Scheme 1 and Scheme 2, the various substituents on the starting compounds (e.g., compounds S1 and S3) are as defined for Formula I. However, it should also be appreciated that chemical derivatization and/or functional group interconversion, can be used to further modify of any of the compounds of Scheme 1 and Scheme 2 in order to provide the various compounds of Formula I.

In some embodiments, the prodrugs are prepared by reacting protected amino acids (e.g., N-protected amino acids) with relevant compounds, e.g., compounds having an —OH group at the R⁶ position. Guidance is provided in Examples 63 and 64 of International patent application PCT/US2022/021609, illustrating synthesis of amino acid prodrugs as well as knowledge of general procedures available in the art for producing such prodrugs (see, e.g., Vale et al., 2018, Molecules. 23(9):2318; Beauchamp et al., 1992, Antiviral Chemistry & Chemotherapy 3(3):157-164; incorporated herein by reference).

Other compounds of the disclosure can be synthesized using the synthetic routes above and adapting chemical synthetic procedures available to the skilled in the art. Exemplary methods of synthesis of the various compounds described herein are provided in International patent publication WO2021/062030, and International patent application no. PCT/US2022/021609, all of which are incorporated herein by reference in their entireties.

### 5.4. PHARMACEUTICALLY ACCEPTABLE SALTS

In some embodiments, the PKC modulating compounds are in free form or where appropriate as pharmaceutically acceptable salt. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

Pharmaceutically acceptable salts of the compounds of this invention include those derived from suitable inorganic and organic acids and bases. In some embodiments, pharmaceutically acceptable salt of the compounds herein can be prepared during final isolation and purification of the compounds. For example, a pharmaceutically acceptable salt of the compounds herein can be prepared by (1) reacting the compound in free base form with a suitable organic or inorganic acid, and (2) isolating the salt thus formed. Examples of pharmaceutically acceptable, nontoxic acid addition salts are salts formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Base addition salts can be prepared by (1) reacting the compound, such as the purified compound, in its acid form with a suitable organic or inorganic base, and (2) isolating the salt thus formed. Salts derived from appropriate bases include alkali metal, alkaline earth metal, ammonium and N⁺(C₁-C₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

### 5.5. FORMULATIONS AND ADMINISTRATION

In some embodiments, the pharmaceutical compositions of the therapeutic agents can be formulated by standard techniques using one or more physiologically acceptable carriers or excipients. Suitable pharmaceutical carriers are described herein and in Remington: The Science and Practice of Pharmacy, 21^{st} Ed. (2005). The PKC activating compounds and their physiologically acceptable salts, hydrates and solvates can be formulated for administration by any suitable route, including, among others, topically, nasally, orally, parenterally, rectally or by inhalation. In some embodiments, the compounds and pharmaceutical compositions thereof are administered by intradermal, subdermal, intravenous, intramuscular, intranasal, intracerebral, intratracheal, intraarterial, intraperitoneal, intravesical, intrapleural, intracoronary or intratumoral injection, such as with a syringe or other devices. Transdermal administration is also contemplated.

For oral administration, a pharmaceutical composition can take the form of, for example, a tablet or a capsule prepared by conventional means with a pharmaceutically acceptable excipient. Tablets and capsules comprising the active ingredient can be prepared together with excipients such as: (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; (c) binders, e.g., magnesium aluminum silicate, starch paste, gelatin, tragacanth, methylcellulose, sodium carboxymethylcellulose, polyvinylpyrrolidone and/or hydroxypropyl methylcellulose; (d) disintegrants, e.g., starches (including potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. The compositions are prepared according to conventional mixing, granulating or coating methods.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The PKC activating agents can be formulated for parenteral administration, for example by bolus injection or continuous infusion. Formulations for injection can be presented in unit dosage form, for example, in ampoules or in multi-dose containers, with an optionally added preservative. Injectable compositions can be aqueous isotonic solutions or suspensions. In some embodiments for parenteral administration, the PKC activating compounds can be prepared with a surfactant, such as Cremaphor, or lipophilic solvents, such as triglycerides or liposomes. The compositions may be sterilized and/or contain adjuvants, such as preserving, stabilizing, wetting or emulsifying agents, solution promoters, salts for regulating the osmotic pressure and/or buffers. Alternatively, the PKC activating compound can be in powder form for reconstitution with a suitable vehicle, for example, sterile pyrogen-free water, before use. In addition, they may also contain other therapeutically effective substances.

For administration by inhalation, the therapeutic agent may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a PKC activating compound with a carrier. Preferred carriers include absorbable pharmacologically acceptable solvents to assist passage through the skin of the subject. For example, transdermal devices are in the form of a bandage or patch comprising a backing member, a reservoir containing the therapeutic agent optionally with carriers, optionally a rate controlling barrier to deliver the compound to the skin of the host at a controlled and predetermined rate over a prolonged period of time, and a means to secure the device to the skin. Matrix transdermal formulations may also be used.

Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

In some embodiments, the PKC activating compounds can also be formulated as a rectal composition, for example, suppositories or retention enemas, for example, containing conventional suppository bases, for example, cocoa butter or other glycerides, or gel forming agents, such as carbomers.

In some embodiments, the PKC activating compound can be formulated as a depot preparation. Such long-acting formulations can be administered by implantation (for example, subcutaneously or intramuscularly) or by intramuscular injection. The compounds for use as adjuvants can be formulated with suitable polymeric or hydrophobic materials (for example as an emulsion in an acceptable oil), ion exchange resins, biodegradable polymers, or as sparingly soluble derivatives, for example, as a sparingly soluble salt.

In some embodiments, the carrier is a cyclodextrins, such as to enhance solubility and/or bioavailability of the compounds herein. In some embodiments, the cyclodextrin for use in the pharmaceutical compositions can be selected from α-cyclodextrin, β-cyclodextrin, γ-cyclodextrin, derivatives thereof, and combinations thereof. In particular, the cyclodextrin is selected from β-cyclodextrin, γ-cyclodextrin, derivatives thereof, and combinations thereof.

In some embodiments, the compounds can be formulated with a cyclodextrin or derivative thereof selected from carboxyalkyl cyclodextrin, hydroxyalkyl cyclodextrin, sulfoalkylether cyclodextrin, and an alkyl cyclodextrin. In various embodiments, the alkyl group in the cyclodextrin is methyl, ethyl, propyl, butyl, or pentyl.

In some embodiments, the cyclodextrin is α-cyclodextrin or a derivative thereof. In some embodiments, the α-cyclodextrin or derivative thereof is selected from carboxyalkyl-α-cyclodextrin, hydroxyalkyl-α-cyclodextrin, sulfoalkylether-α-cyclodextrin, alkyl-α-cyclodextrin, and combinations thereof. In some embodiments, the alkyl group in the α-cyclodextrin derivative is methyl, ethyl, propyl, butyl, or pentyl.

In some embodiments, the cyclodextrin is β-cyclodextrin or a derivative thereof. In some embodiments, the β-cyclodextrin or derivative thereof is selected from carboxyalkyl-β- cyclodextrin, hydroxyalkyl-β-cyclodextrin, sulfoalkylether-β-cyclodextrin, alkyl-β-cyclodextrin, and combinations thereof. In some embodiments, the alkyl group in the β-cyclodextrin derivative is methyl, ethyl, propyl, butyl, or pentyl.

In some embodiments, the β-cyclodextrin or a derivative thereof is hydroxyalkyl-β-cyclodextrin or sulfoalkylether-β-cyclodextrin. In some embodiments, the hydroxyalkyl-β-cyclodextrin is hydroxypropyl-β-cyclodextrin. In some embodiments, the sulfoalkylether-β-cyclodextrin is sulfobutylether-β-cyclodextrin. In some embodiments, β-cyclodextrin or a derivative thereof is alkyl-β-cyclodextrin, in particular methyl-β-cyclodextrin. In some embodiments using methyl-β-cyclodextrin, the β-cyclodextrin is randomly methylated β-cyclodextrin.

In some embodiments, the cyclodextrin is γ-cyclodextrin or a derivative thereof. In some embodiments, the γ-cyclodextrin or derivative thereof is selected from carboxyalkyl-γ-cyclodextrin, hydroxyalkyl-γ-cyclodextrin, sulfoalkylether-γ-cyclodextrin, and alkyl-γ-cyclodextrin. In some embodiments, the alkyl group in the γ-cyclodextrin derivative is methyl, ethyl, propyl, butyl, or pentyl. In some embodiments, the γ-cyclodextrin or derivative thereof is hydroxyalkyl-γ-cyclodextrin or sulfoalkylether-γ-cyclodextrin. In some embodiments, the hydroxyalkyl-γ-cyclodextrin is hydroxypropyl-γ-cyclodextrin.

When used in a formulation with the PKC activating compound, the cyclodextrin can be present at about 0.1 w/v to about 30% w/v, about 0.1 w/v to about 20% w/v, about 0.5% w/v to about 10% w/v, or about 1% w/v to about 5% w/v. In some embodiments, the cyclodextrin is present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, or about 30% w/v or more.

### 5.6. EFFECTIVE AMOUNT AND DOSING

In some embodiments, a pharmaceutical composition of the PKC activating compound is administered to a subject, preferably a human, at an effective dose to stimulate an immune response to an antigenic material.

The dosage of agents can take into consideration, among others, the species of warm-blooded animal (mammal), the body weight, age, type of antigenic material, the desired level of immune response, the form and route of administration. The size of the dose also will be determined by the existence, nature, and extent of any adverse effects that accompany the administration of a particular PKC activating compound in a particular subject.

In some embodiments, the diterpenoid PKC activating compound can be administered in a dose in the range from about 0.0001 mg per kg of subject weight (0.0001 mg/kg) to about 1000 mg/kg or from about 0.001 mg per kg of subject weight (0.001 mg/kg) to about 1000 mg/kg. In some embodiments, the dose is in the range of about 0.0001 mg/kg to about 800 mg/kg. In some embodiments, the dose is in the range of about 0.001 mg/kg to about 500 mg/kg. In some embodiments, the dose is in the range of about 1 mg/kg to about 500 mg/kg. In some embodiments, the dose is about 2 mg/kg to about 250 mg/kg. In another embodiment, the dose is about 5 mg/kg to about 100 mg/kg. In another embodiment, the dose is about 5 mg/kg to about 100 mg/kg. In some embodiments, the dose is about 0.0001 mg/kg, 0.001 mg/kg, 0.01 mg/kg, 0.05 mg/kg, 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/kg, 10 mg/kg, 20 mg/kg, 40 mg/kg, 50 mg/kg, 100 mg/kg, 200 mg/kg or 500 mg/kg.

When administered concurrently with an antigenic material, the diterpenoid PKC modulator can be administered separately at the same time as the antigenic material, by the same or different routes, or administered in a single composition by the same route.

In some embodiments, the amount and frequency of administration of the antigenic material can use standard dosages and standard administration frequencies used for the particular antigenic material.

In some embodiments, where administration of the antigenic material is to a localized site, for example, subcutaneous, the dosages can be the dosages used for systemic administration, such as dosages used for intravenous, intramuscular, and intraperitoneal administration. In some embodiments, the dose for localized administration, e.g., subcutaneous administration, is higher than those used for systemic administration. In some embodiments, the administered dose is sufficient for the intended effect, for example stimulating an immure response to an antigenic material. In some embodiments, intratumoral administration is done once, twice, three times, four times, five time or up to six times or more, where each administration is separated in time, for example, until the desired immune response is achieved.

The following examples are provided to further illustrate the methods of the present disclosure, and the compounds and compositions for use in the methods. The examples described are illustrative only and are not intended to limit the scope of the invention in any way.

### 6. EXAMPLES

### Example 1: Examination of NF-κB Activation of PKC Activating Compounds

The PKC activator compounds were evaluated for their effect on NFκB expression using a luciferase reporter gene expression system. (The indicators “K-101,” “K101” and “K-101A” may be used interchangeably in this disclosure and appended figures).

A HeLa-NFκB luciferase reporter cell line (Wuxi Biology) was used to assess a group of PKC activator compounds for their ability to activate NFκB-driven transcription. Two sets of cells at 20000 cells/well were plated, grown overnight, and then treated for 16 h at 37 °C with DMSO, 6 doses of each compound, or TNF-α (final concentration of 10 ng/mL, positive control) in triplicates. One set of cells was assayed for luciferase activity (Promega, Cat #E1500) and the other set was assayed to determine the number of viable cells (for cell number normalization) using CellTiter-Glo (CTG) kit (Promega, Cat #G7570) according to manufacture's procedures. Normalized data (luciferase value/CTG value) were analyzed using GraphPad Prism5 to calculate EC50 for each compound (see Table 3).

Results. TNF-α (10 ng/mL) activated NFκB luciferase reporter gene expression near 10-fold above DMSO control. The normalized ratio of luciferase/CTG is from 0.15 to 0.2. The magnitude of NFκB activation stimulated by TNF-α (10 ng/mL) was very similar to the maximal stimulation by the PKC activator compounds.

TABLE 3

| | NFκB-luc HeLa cells |
|---|---|
| Compound ID | EC50 (μM) |
| K-101 | G |
| K101-C1301 | E |
| K101-C1302 | C |
| K101-C1303 | G |
| K101-C1304 | D |
| K101-C1308 | E |
| K101-C1318 | F |
| K101-C1319 | E |
| K101-C1321 | F |
| K101-C1324 | C |
| K101-C1327 | D |
| K101-C1329 | E |
| K101-C1333 | B |
| K101-C1342 | E |
| K101-C1345 | C |
| K101-C1347 | B |
| K101-C134801 | C |
| K101-C134802 | B |
| K101-C134901 | F |
| K101-C134902 | E |

The letters in Table 3 represent: A = ≤0.001 uM; B = >0.001 uM and ≤0.01 uM; C = >0.01 uM and ≤0.03 uM; D = >0.03 uM and ≤0.06 uM; E = >0.06 uM and ≤0.1 uM; F = >0.1 uM and ≤0.2 uM; G = >0.2 uM and ≤0.6 uM; H = >0.6 uM and ≤1.0 uM; I = >1.0 uM and ≤2.0 uM; J = >2 uM and ≤5 uM; K = >5 uM and ≤10 uM; L = >10 uM and ≤30 uM; and M = >30 uM.

These PKC activator compounds were very potent to activate NFκB reporter gene transcription: 3 compounds with EC50 less than 0.01M, 13 compounds with EC50 less than 0.1 μM, and 4 compounds with EC50 greater than 0.1 M but less than 1 M.

To assess if activation of PKC is required for these compounds to activate NFκB, several PKC inhibitors including broad-spectrum and conventional PKC inhibitors were tested. In the assay described above, PKC inhibitors were added at the same time as the PKC activator compounds.

Sotrastaurin, a PKC inhibitor targeting both conventional and novel classes of PKC, and G66983, targeting all PKCs (conventional, novel, atypical, and PKCμ), were able to completely block NFκB activation induced by a number of PKC activator compounds (K101A, K101-C1302, K101-C1345, K101-C1347, K101-C134801, and K101-C134802).

However, Gö6976, a PKC inhibitor targeting mainly the conventional PKC isoforms, was unable to block NFκB activation stimulated by these PKC activator compounds (data not shown).

Conclusions: The compounds tested are PKC activators. In addition, the PKC activator compound-mediated NFκB activation is not via the conventional PKC isoforms and most likely via the novel PKC isoforms.

### Example 2: Stimulation of Cytokine/Chemokine in Human Peripheral Blood Mononuclear Cell (PBMC) by PKC Activators

This study evaluated the effect of PKC activator compounds on expression of cytokines/chemokines in human PMBC cells.

Materials and methods: 100 million frozen human PBMC cells were purchased from Allcells (100 million/vial). The cells were thawed and recovered for one day before 1.5-1.8 million cells in 900 μl RPMI1640 with 10% heat-inactivated FBS were seeded in each well of 24-well plates. Various compounds in DMSO stock (500×) were diluted 50-fold with the same media to make 10× compound stock for most compounds. 1 μL of 5 mg/mL PMA was diluted to 99 μL of the media to make 50 μg/mL PMA. For PMA with ionomycin treatment condition, 1.5 μL of 50 μg/mL PMA and 3 μL of 500 μg/mL ionomycin were added to 145.5 μL of the media to make 10× compound stock. Sources of the compounds are as follows: PMA from Abcam (Abcam #ab120297); Ionomycin from Sigma (Sigma #I0634); Resiquimod from Sellect Chem. (Selleckchem #S8133); and LPS from Sigma (Sigma #L6143).

100 μl of the diluted compound (10×) was added to the cells and the plates were incubated at 37 °C, 5% CO₂ incubator for 4 hours. Each treatment condition was performed in duplicates. After treatment, mRNA was extracted from cells and samples from duplicates were combined for TaqMan gene expression assays (ThermoFisher) in triplicates. Expression of 19 genes (IL-1β, IL-2, IL-4, IL-6, IL-8, IL-10, IL-13, IL-18, IL-12p70/IL-12A, IL-12p40/IL-12B, IFN-α, IFN-γ, GM-CSF/CSF2, CCL2/MCP-1, MIP1a/CCL3, TNF-α, MX1, OAS1, ISG15) along with two housekeeping gene controls, 18S and GAPDH, was determined. Target gene expression in each sample was determined by relative quantification (RQ) using the comparative Ct (ΔΔCT) method. This method measures the Ct difference (ΔCT) between target gene and the housekeeping genes (18S & GAPDH), then compares the ΔCT values of treated samples to DMSO-treated control samples. The mean ΔCT value of the DMSO-treated control from two independent DMSO-treated control samples was used. The relative expression level of target gene was calculated by using the equation:

relative expression=2^{-ΔΔCT}

Results and discussions. The results of relative expression of 19 genes upon treatment of various compounds for 4 hours in human PBMC cells are shown in Table 4A-4C.

TABLE 4A

| | | Gene Relative Expression | | | | | |
|---|---|---|---|---|---|---|---|
| Treatment | Concentration | IFNγ | GM-CSF | IL-1β | IL-2 | IL-6 | IL-8 |
| DMSO | | 1 | 1 | 1 | 1 | 1 | 1 |
| PMA (ng/mL) + | 50 + 1 | 23.93 | 17.00 | 4.98 | 2.98 | 4.02 | 1.94 |
| ionomycin (μg/mL) | | | | | | | |
| Resiquimod (μM) | 1 | 1.28 | 8.79 | 101.63 | 1.21 | 487.14 | 9.87 |
| LPS (ng/ml) | 50 | 4.98 | 16.24 | 66.54 | 1.20 | 431.91 | 7.72 |
| K-101 (μM) | 0.3 | 4.52 | 5.69 | 1.82 | 5.59 | 0.77 | 0.62 |
| | 1 | 13.46 | 16.75 | 1.82 | 7.71 | 4.83 | 0.35 |
| | 3 | 18.11 | 11.22 | 3.15 | 2.71 | 5.41 | 1.13 |
| K101-C1347 | 0.01 | 1.69 | 1.59 | 0.59 | 3.06 | 1.17 | 0.71 |
| (μM) | 0.03 | 3.82 | 8.38 | 2.26 | 7.78 | 0.86 | 0.54 |
| | 0.10 | 18.08 | 16.06 | 3.00 | 3.29 | 6.72 | 0.60 |
| K101-C134802 | 0.01 | 1.46 | 1.00 | 0.38 | 1.32 | 0.57 | 0.44 |
| (μM) | 0.03 | 1.49 | 1.88 | 0.94 | 3.59 | 1.23 | 0.66 |
| | 0.10 | 11.33 | 21.01 | 3.00 | 4.98 | 6.15 | 0.44 |
| K101-C134801 | 0.03 | 1.62 | 1.89 | 0.54 | 1.71 | 0.74 | 0.80 |
| (μM) | 0.10 | 3.47 | 7.74 | 1.58 | 7.65 | 1.14 | 0.53 |
| | 0.30 | 18.57 | 10.50 | 1.53 | 3.41 | 4.85 | 0.59 |

TABLE 4B

| | | Gene Relative Expression | | | | | |
|---|---|---|---|---|---|---|---|
| | | | | | MIP1a | | |
| Treatment | Concentration | IL-10 | IL-13 | IL-18 | (CCL3) | MX1 | OAS1 |
| DMSO | | 1 | 1 | 1 | 1 | 1 | 1 |
| PMA (ng/mL) + | 50 + 1 | 0.06 | 85.82 | 0.08 | 6.35 | 0.37 | 0.38 |
| ionomycin (μg/mL) | | | | | | | |
| Resiquimod (μM) | 1 | 5.65 | 3.52 | 1.33 | 47.71 | 17.00 | 12.66 |
| LPS (ng/ml) | 50 | 16.76 | 4.59 | 0.65 | 40.26 | 89.81 | 51.01 |
| K-101 (μM) | 0.3 | 0.37 | 39.71 | 0.07 | 2.95 | 0.53 | 0.51 |
| | 1 | 0.10 | 170.67 | 0.03 | 5.90 | 1.31 | 0.88 |
| | 3 | 0.07 | 80.77 | 0.05 | 3.72 | 0.58 | 0.41 |
| K101-C1347 | 0.01 | 1.12 | 3.10 | 0.26 | 1.65 | 0.84 | 0.74 |
| (μM) | 0.03 | 0.41 | 52.65 | 0.06 | 2.82 | 1.01 | 0.78 |
| | 0.10 | 0.09 | 101.38 | 0.06 | 5.73 | 0.99 | 0.69 |
| K101-C134802 | 0.01 | 1.59 | 0.37 | 0.63 | 1.29 | 0.77 | 0.85 |
| (μM) | 0.03 | 0.86 | 5.87 | 0.19 | 1.67 | 0.65 | 0.55 |
| | 0.10 | 0.12 | 121.59 | 0.05 | 4.95 | 1.06 | 0.71 |
| K101-C134801 | 0.03 | 1.27 | 0.40 | 0.47 | 1.86 | 0.65 | 0.64 |
| (μM) | 0.10 | 0.48 | 47.45 | 0.09 | 2.39 | 0.72 | 0.55 |
| | 0.30 | 0.06 | 78.90 | 0.04 | 3.37 | 0.57 | 0.42 |

TABLE 4C

| | | Gene Relative Expression | | | | | | |
|---|---|---|---|---|---|---|---|---|
| Treatment | Concentration | IL-12p70 | IL-12p40 | IL-4 | CCL2 | TNF-α | ISG15 | IFN-α |
| DMSO | | 1 | 1 | 1 | 1 | 1 | 1 | 1 |
| PMA (ng/mL) + | 50 + 1 | 0.69 | 1.91 | 1.85 | 0.26 | 5.32 | 1.27 | 0.73 |
| ionomycin (μg/mL) | | | | | | | | |
| Resiquimod (μM) | 1 | 2.48 | 786.39 | ND | 3.73 | 7.38 | 14.75 | 1.47 |
| LPS (ng/ml) | 50 | 4.99 | 113.45 | 1.06 | 0.00 | 2.98 | 125.05 | 1.71 |
| K-101 (μM) | 0.3 | 1.03 | ND | 3.18 | 7.65 | 4.51 | 0.93 | 0.88 |
| | 1 | 0.73 | 0.82 | 3.83 | 0.64 | 4.81 | 1.54 | 0.76 |
| | 3 | 0.71 | 0.56 | 2.29 | 2.79 | 4.20 | 1.21 | 0.52 |
| K101-C1347 | 0.01 | 1.03 | 0.89 | 0.84 | 5.58 | 2.39 | 1.01 | 0.78 |
| (μM) | 0.03 | 0.87 | 0.32 | 2.54 | 3.59 | 4.48 | 1.33 | 0.46 |
| | 0.10 | 0.76 | 0.22 | 2.77 | 0.38 | 6.25 | 1.54 | 0.31 |
| K101-C134802 | 0.01 | 1.05 | 0.34 | ND | 1.81 | 1.34 | 0.94 | 0.73 |
| (μM) | 0.03 | 0.96 | 0.38 | 1.19 | 6.53 | 2.91 | 0.97 | 1.21 |
| | 0.10 | 0.74 | ND | 2.78 | 0.40 | 6.52 | 1.57 | 0.80 |
| K101-C134801 | 0.03 | 1.02 | 0.74 | ND | 4.55 | 1.38 | 0.97 | 1.66 |
| (μM) | 0.10 | 0.93 | 0.45 | 1.61 | 3.12 | 4.04 | 1.16 | 0.73 |
| | 0.30 | 0.61 | 0.69 | 2.57 | 2.09 | 4.13 | 1.14 | 0.95 |

Expression of 19 genes including cytokines, chemokines, interferons and interferon-stimulated genes (ISGs) was determined upon treatment of the various compounds.

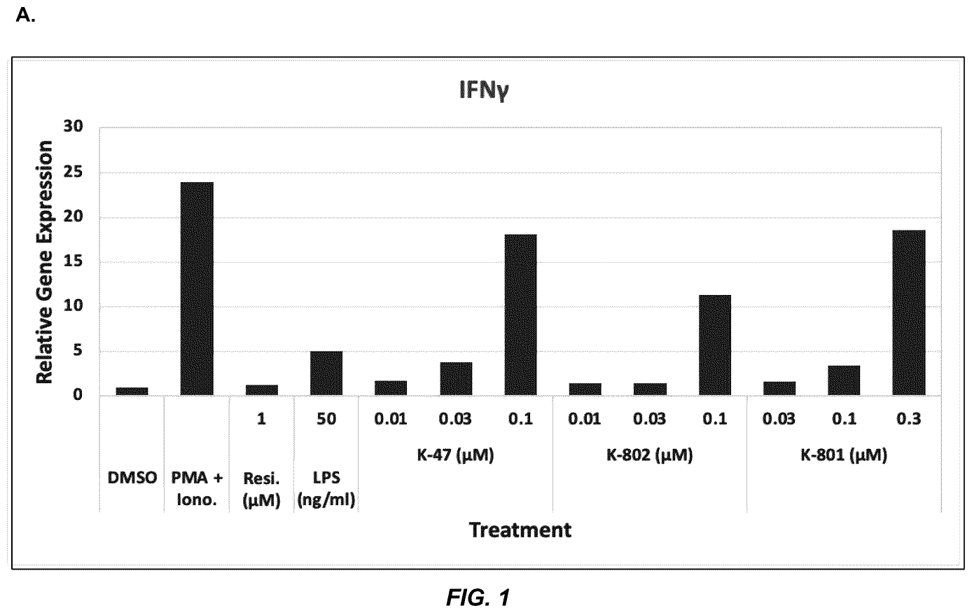

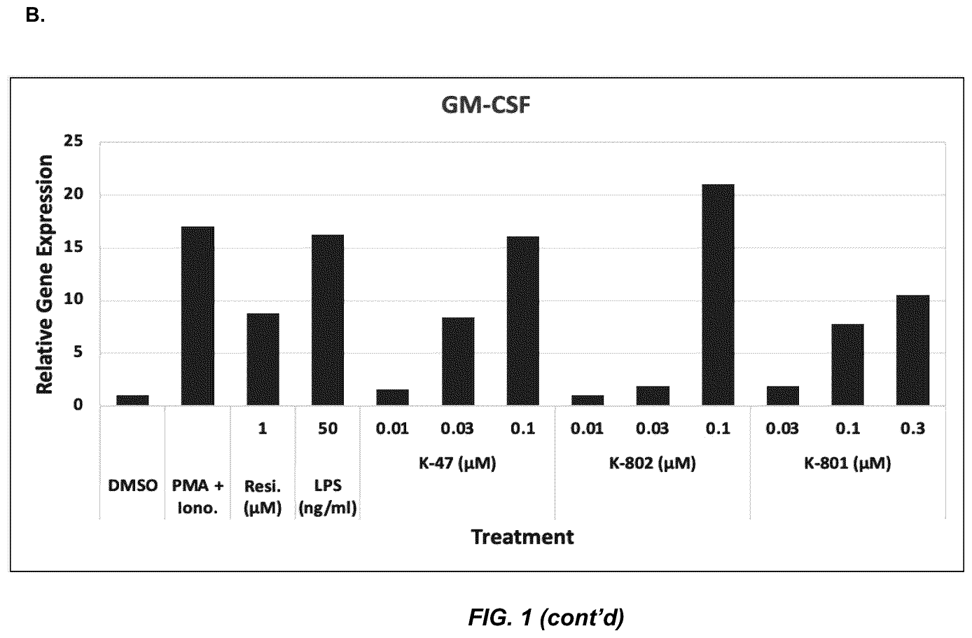

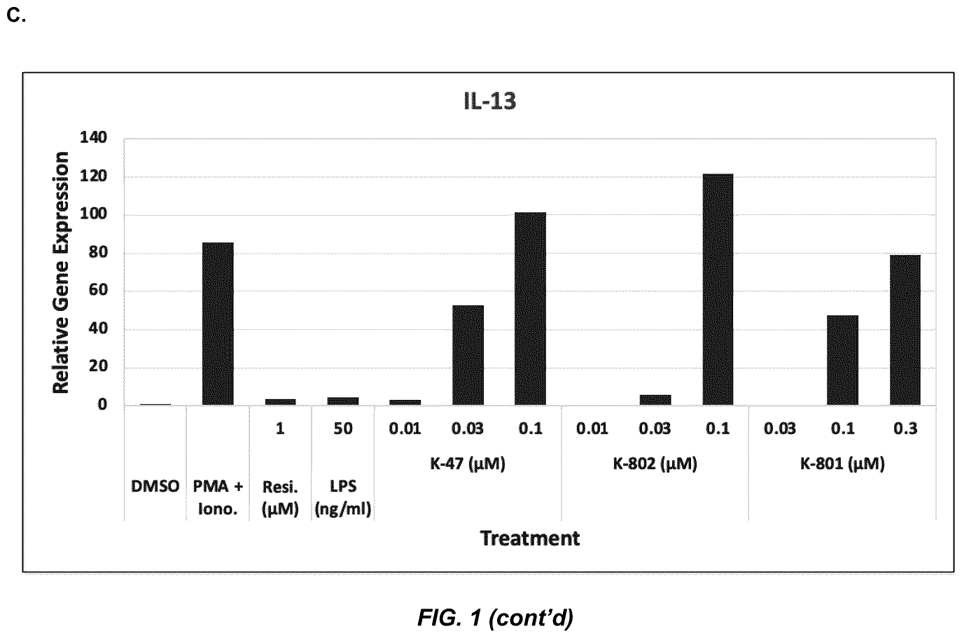

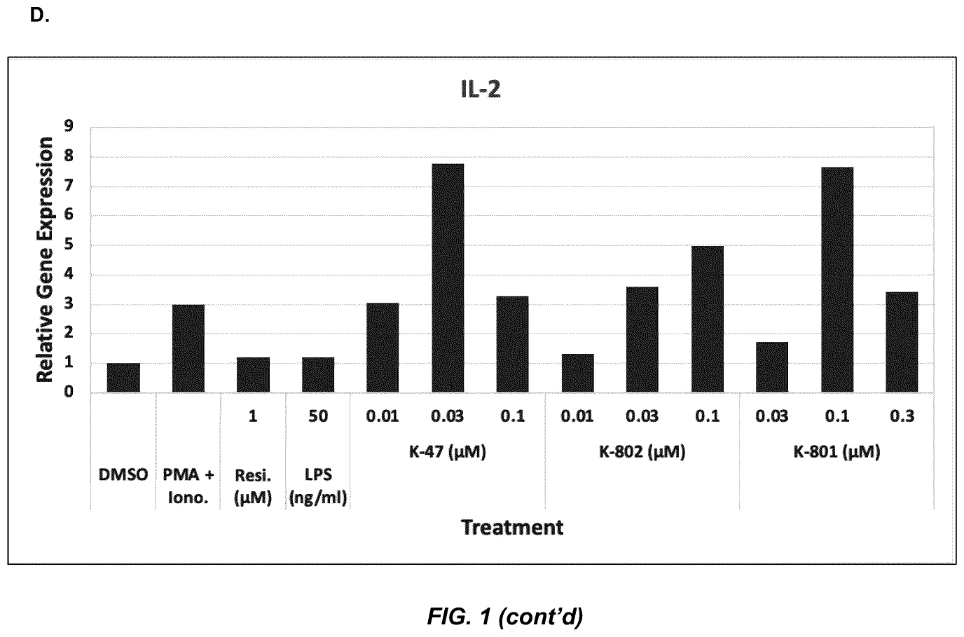

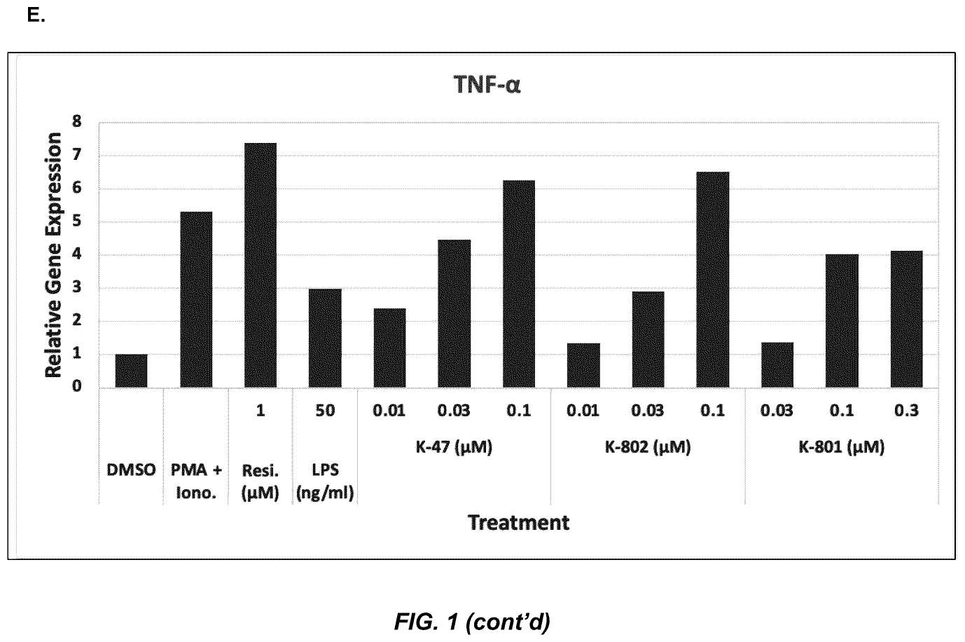

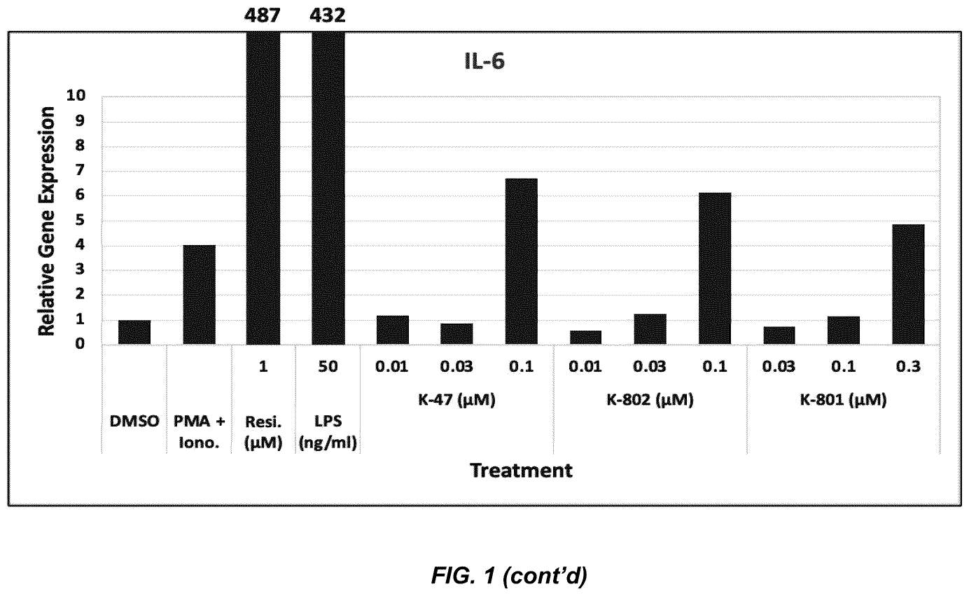

Among them, IFNγ (FIG. 1A), GM-CSF (FIG. 1B), and IL-13 (FIG. 1C) expressions were strongly induced in a dose-dependent manner (up to >10-fold increase over DMSO) by the PKC activator compounds. IFNγ and GM-CSF are prototypical anti-tumor cytokines and immune stimulants. IL-13 is one of the type-2 cytokines and a mediator of allergic inflammation and recent data suggest that IL-13 in the epidermis promotes barrier integrity and protects against carcinogenesis.

IL-1β, IL-2, IL-6, MIP1a (CCL3), TNF-α was induced more than 4-fold in certain compound-treated samples compared to the DMSO control samples. Some of these cytokines/chemokines mediate pro-inflammatory responses and anti-tumor immunity by activating immune cells to kill tumor cells directly or recruiting anti-tumor immune cells to the tumor sites, or involving in immune cell proliferation. IL-2 and TNF-α stimulation data are shown in FIG. 1D and FIG. 1E, respectively.

However, it is worth noting that IL-6, induced moderately by the PKC activator compounds, was strongly/excessively induced by the TLR agonists such as resiquimod or LPS (>400-fold) (FIG. 1F).

High levels of IL-6 can trigger uncontrolled inflammation leading to cytokine release syndrome (CRS). Thus, the PKC activator compounds may have a much lower risk than TLR agonists to induce CRS.

CCL2 was moderately induced by lower dose of PKC activators, however, it was slightly suppressed by higher dose of PKC activators. Inhibition of CCL2 and its receptor CCR2 has been implicated in enhancing efficacy of checkpoint inhibitors (anti-PD-1/PD-L1).

Genes with changes less than 4-fold when treated with the PKC activator compounds include: IL-8, MX1, OAS1, IL-12p70, IL-12p40, IL-4, ISG15, and IFN-α. In contrast, a few genes including interferon-stimulated genes (ISGs) (e.g. MX1, OAS1, IL-12p40, and ISG15) were highly induced by TLR agonists (resiquimod or LPS) as expected.

While most cytokines were induced, IL-10 and IL-18 were suppressed after treatment of the PKC activator compounds. IL-10 is an immunomodulatory cytokine that is frequently upregulated in various types of cancers. While TLR agonists induced IL-10, PKC activators suppressed IL-10 significantly in a dose-dependent manner which may lead to reduced immune suppression and enhanced antitumor immune response. IL-18 is an inflammatory cytokine, synthesized as a precursor protein which is then activated by proteolytic processing. Due to complex regulation of IL-18 by multiple mechanisms (transcriptional, post-translational, and decoy receptors), its role in cancer as an immunostimulatory or immunosuppressive cytokine is still controversial.

### Example 3: Stimulation of IFN-γ Production in Mouse Splenocytes

Production of IFN-γ in response to infection or other stimuli is a hallmark of innate and adaptive immunity. To measure the effect of direct immune stimulation, freshly isolated splenocytes from three C₅₇BL/6J mice were seeded in ELISpot plates pre-coated with anti-IFN-γ antibody (Mabtech, #3321-4AST-2) at 1 million splenocytes per well in OptiVitro® T Cell Serum-free Medium (Excellbio, Cat #TE000-N021). The splenocytes were then incubated with medium only or different concentrations of K101-C134801 for 24 hours. Detection of the IFN-γ spot-forming cells was performed according to the manufacture's protocol. Briefly, cells were removed from the ELISpot plates. After washing with PBS, the ELISpot plates were incubated with 1 g/ml of the R4-6A2-biotin detection antibody for 2 hours at room temperature (RT) and then with streptavidin-ALP (1:1000) at RT for 1 hour. BCIP/NBT chromogen substrate solution was added to the plates until distinct spots emerged. The spots (IFN-γ-secreting cells) were counted using an AID ELISPOT reader.

The results are shown in the table below. K101-C134801 stimulated IFN-γ production from mouse splenocytes at concentration as low as 60 nM. At 200 nM stimulation, IFN-γ-secreting cells could not be accurately numerated due to saturation of the signals. Although the signal was saturated at 200 nM stimulation, the results indicated a trend of dose-response.

IFN-γ secreting cells/million splenocytes

| | | K101-C134801 | |
|---|---|---|---|
| | Medium | 60 nM | 200 nM |
| Mouse 1 | 36 | 245 | saturated |
| Mouse 2 | 13 | 137 | saturated |
| Mouse 3 | 28 | 110 | saturated |

### Example 4: In Vivo Mouse Model to Assess Adjuvant Effects

To measure adjuvant effect, humoral immune responses induced by an immunogen/antigen from an invading pathogen with or without test adjuvants were evaluated.

As a proof-of-concept in vivo study in mice, SARS-CoV-2 spike receptor-binding domain (RBD) (a weak antigen by itself) was used as the antigen, alone or in combination with selected compounds from Table 1 and/or Table 2. Female, 6-8 weeks old, SPF BALB/c mice were purchased and underwent a general health examination upon arrival. Once the mice were acclimatized to the new environment, healthy mice were weighed and assigned to one of seven groups based on similar body weights. The mice were immunized with vehicle, antigen, test compound, or a combination of the antigen and the test compound at day 0 (the day of first injection) and day 14 (two weeks later), via intramuscular injections. The study design is shown below:

| | | | Immunization | |
|---|---|---|---|---|
| Group | Animals | Test article (Dose/Volume) | Dosing route | Dosing regimen |
| 1 | 5 | Vehicle | Intramuscular | Days 0, 14 |
| 2 | 5 | SARS-CoV-2 Spike RBD (5 μg/20 μL) | injection | |
| 3 | 5 | K101-C134802C2003 (3 μg/20 μL) | | |
| 4 | 5 | SARS-CoV-2 Spike RBD (5 μg/20 μL) + | | |
| | | K101-C134802C2003 (1.5 μg/20 μL) | | |
| 5 | 5 | SARS-CoV-2 Spike RBD (5 μg/20 μL) + | | |
| | | K101-C134802C2003 (3 μg/20 μL) | | |
| 6 | 5 | SARS-CoV-2 Spike RBD (5 μg/20 μL) + | | |
| | | K101-C134801 (3 μg/20 μL) | | |
| 7 | 5 | SARS-CoV-2 Spike RBD (5 μg/20 μL) + | | |
| | | K101-C1327 (2.5 μg/20 μL) | | |

SARS-CoV-2 Spike RBD recombinant protein was purchased from SinoBiological (#40592-VNAH) and dissolved at a concentration of 0.25 mg/mL in PBS, pH7.4. The test adjuvant compounds K101-C134802C2003, K101-C134801, and K101-C1327 were formulated in 10% DMSO/50% PEG400/40% H₂O. The antigen SARS-CoV-2 Spike RBD and the test adjuvant compounds were injected intramuscularly, into the same muscle of each mouse.

Mice were monitored twice a week for body weights, health conditions, and mortality during the entire in-life stage. All animal procedures were conducted in accordance with the guidelines of the Animal Care and Use Committee. The body weights of the mice in all groups remained relatively stable; no body weight loss greater than 10% was observed. There was no local toxicity, although certain local reactions such as swelling and/or lumps were observed at the injection sites of the test compounds. Most of the local reactions were mild and disappeared within a few days.

Two weeks after the last immunization (day 28, last day of the in-life portion of the study), blood samples were collected and centrifuged at 3,000 g, 4 °C for 10 minutes. Sera were separated after centrifugation and stored at −80 °C.

SARS-CoV-2 Spike RBD-specific IgG in mouse serum was measured by ELISA. Briefly, 96-well plates were coated with 50 μL of 1.0 μg/mL SARS-CoV-2 Spike RBD recombinant protein overnight at 4 °C, washed, and blocked with 3% BSA for 1 hour. Mouse serum samples were serially diluted with assay buffer (Biolegend, #421203), added to the SARS-CoV-2 Spike RBD-coated plates in duplicates of 8 concentrations, and incubated for 1-2 hours at room temperature. The plates were washed and incubated with 1:5000 HRP-conjugated goat anti-mouse antibodies (Abcam, #ab97023) for 45-60 minutes at room temperature. After washing, TMB substrate solutions (Biolegend, #421101) were added and incubated for 10-20 minutes in the dark before adding stop solutions (Biolegend, #423001). Finally, the optical density (OD) was determined using a microplate reader (Molecular Devices, SpectraMax M2e) at 450 nm. The data were analyzed using a four-parameter logistic model and the endpoint antibody titer, the highest dilution having an OD value that is three times greater than the background, was determined. The endpoint antibody titer of 50 was the lower limit of detection of the assay.

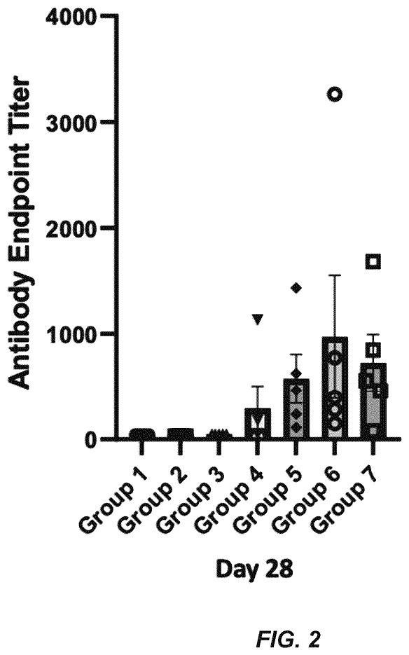

Assessments of the SARS-CoV-2 Spike RBD-specific IgG endpoint titers are summarized in FIG. 2. The titer of SARS-CoV-2 Spike RBD-specific IgG in mice was measured on day 28. As expected, no SARS-CoV-2 Spike RBD-specific IgG was detected in mice from group 1 (vehicle) or group 3 (test compound K101-C134802C2003 alone without immunogen). While no SARS-CoV-2 Spike RBD-specific IgG was detected in group 2 even though this group received two immunizations of SARS-CoV-2 Spike RBD, SARS-CoV-2 Spike RBD-specific antibodies with different endpoint titers were detected on day 28 in mouse serum samples from groups 4-7. Mice from groups 4-7 were immunized twice with SARS-CoV-2 Spike RBD along with different test adjuvant compounds. The results indicate that these test adjuvant compounds indeed can enhance the generation of antigen-specific antibody. In addition, a trend of dose-dependency was observed in groups 4 and 5 in which different doses of K101-C134802C2003 were tested.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of generating an immune response, comprising administering to a subject an antigenic material, and an adjuvant comprising an effective amount of a PKC activating compound, with the proviso that the antigenic material is not a cancer vaccine or cancer antigen.

2. The method of claim 1, wherein the antigenic material is, or is from a pathogenic virus, bacteria, fungus, or protozoa.

3. The method of claim 2, wherein the antigenic material is, or is from, a virus.

4. The method of claim 3, wherein the antigen material is an attenuated virus, an inactivated virus, a viral protein, a recombinant viral protein, an mRNA vaccine of a viral protein, or a viral toxin.

5. The method of claim 4, wherein the virus is selected from the group consisting of: Severe acute respiratory syndrome (SARS)-related coronavirus, SARS-CoV-2 responsible for COVID-19, MERS-CoV, influenza virus, parainfluenza viruses (PIVs), respiratory syncytial virus (RSV), human metapneumovirus (HMPV), poliovirus, coronavirus, human herpesvirus 6A and 6B (HHV-6A and HHV-6B), human herpesvirus 7 (HHV-7), herpes simplex 1 and 2 (HSV-1 and HSV-2, also known as HHV-1 and HHV-2), human cytomegalovirus (HCMV also known as HHV-5), Epstein-Barr virus (EBV or HHV-4), varicella zoster virus (VZV or HHV-3), human immunodeficiency virus (HIV), Kaposi's sarcoma-associated herpesvirus (KSHV, also known as HHV-8), rabies virus, hepatitis A virus, hepatitis B virus, hepatitis C virus, hepatitis D virus, human papilloma virus (HPV), measles virus, mumps virus, Nipah virus, rubella virus, norovirus, parvovirus, rotavirus, variola virus, smallpox virus, monkeypox virus, West Nile virus, Dengue virus, tick-borne encephalitis virus, Zika virus, yellow fever virus, Ebola virus, BK polyomavirus, and Chikungunya virus (CHIKV).

6. The method of claim 2, wherein the antigenic material is, or is from, bacteria.

7. The method of claim 6, wherein the antigenic material is a bacterium selected from: Bacillus anthracis, Borrelia, Haemophilus, Pneumococcus, Staphylococcus, Streptococcus, Shigella, Klebsiella, Mycobacterium tuberculosis, Clostridium/Clostridioides, pathogenic Escherichia coli, Legionella, Listeria, Salmonella, Neisseria meningitidis, Neisseria gonorrhoeae, Chlamydia, Klebsiella, Vibrio cholera or a bacterial toxin (cholera toxin).

8. The method of claim 2, wherein the antigenic material is, or is from, a fungal pathogen.

9. The method of claim 8, wherein the antigenic material is, or is from, pathogenic Coccidioides, Aspergillus, Histoplasma, Blastomyces, Paracoccidioides, Pneumocystis, Cryptococcus fungal polysaccharide, or mRNA vaccine of a fungal protein.

10. The method of claim 8, wherein the antigenic material is an invasion protein (agglutinin-like sequence 3 (Als3p), aspartyl proteinase-2, glucuronoxylomannan, Gel1p, Crf1p, Pep1p, Cat1p, Sod1p, Dpp5p, RNUp, Mep1p, 1-3 beta-d-glucan (also known as (1→3)-β-D-Glucan, or “BDG”), β-1,3/1,6-glucan, GM, glycolipids: GSL, LGM, β-glucans of S. cerevisiae, Adhesin BAD1, gp 43 (P10), C. posadasii Gel-1 (β 1,3, aspartyl proteinase, phospholipase B or a mannosidase.

11. The method of claim 2, wherein the antigenic material is, or is from, a protozoal pathogen.

12. The method of claim 11, wherein the antigenic material is, or is from, Leishmania, Plasmodium, Naegleria fowleri amoeba, Eimeria, Theileria, Toxoplasma, Babesia, Tritrichomonas, Giardia, Sarcocystis, Neospora, Leishmania, or Babesia.

13. The method of claim 12, wherein the antigenic material comprises a carbohydrate antigen of the protozoal pathogen.

14. The method of claim 2, wherein the antigenic material is an mRNA vaccine encoding a viral, bacterial, fungal, or protozoal antigen protein.

15. The method of any one of claims 1-14, wherein the PKC activating compound is administered prior to, concurrently with, or subsequent to administration of the antigenic material.

16. The method of claim 15, wherein the PKC activating compound is administered concurrently with the antigenic material.

17. The method of claim 15, wherein the PKC activating compound is administered subsequent to administration of the antigenic material.

18. The method of claim 17, wherein the administration of the PKC activating compound is sufficiently spaced to stimulate the immune response to the antigenic material.

19. The method of any one of claims 15-18, wherein the PKC activating compound is subsequently administered one or more times following an initial administration or vaccination.

20. The method of claim 19, wherein the PKC activating compound is administered once every two days, once every 4 days, once every 7 days (per week), once every 14 days (per two weeks), once every 21 days (per three weeks), or once every month.

21. The method of claim 19 or 20, wherein the subsequent administration of the PKC activating compound is in conjunction with administration of a subsequent dose of antigenic material.

22. The method of claim 19 or 20, wherein the subsequent administration of the PKC activating compound is in conjunction with administration without a subsequent dose of antigenic material.

23. The method of any one of claims 1-22, wherein the PKC activating compound is a compound of formula (I): 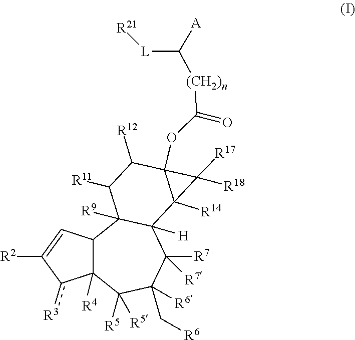
or a pharmaceutically acceptable salt, tautomer, or stereoisomer thereof;
wherein
A is —OH, —C(O)OR′, or —NR¹³R^{13′};
R¹ is H or a M+ counterion;
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 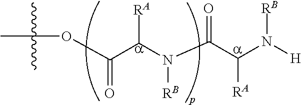
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷ is H, or R⁶′ and R⁷ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR⁸; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

24. The method of claim 23, wherein A is —OH.

25. The method of claim 23, wherein A is —C(O)OR¹, wherein R¹ is H or a M⁺ counterion.

26. The method of claim 23, wherein the compound is a compound of formula (II): 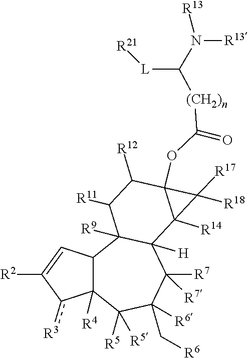
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a1} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}, R^{c1} is H, C₁-C₆alkyl, or two R^{c1} together with the N atom form a 5 to 7 membered heterocyclyl containing 1 to 3 heteroatoms selected from N, O, and S; or
R⁶ is 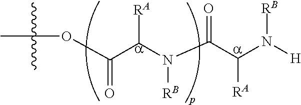
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR⁸; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

27. The method of claim 26, wherein the compound is a compound of formula (II′): 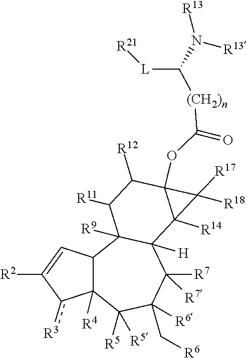
or a pharmaceutically acceptable salt, tautomer, or stereoisomer thereof.

28. The method of claim 26, wherein the compound is a compound of formula (IIa): 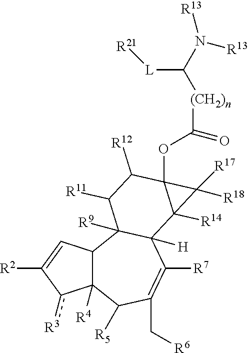
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is 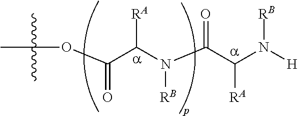
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

29. The method of claim 28, wherein the compound is a compound of formula (IIa′): 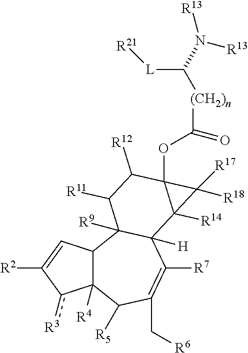
or a pharmaceutically acceptable salt, tautomer, or stereoisomer thereof.

30. The method of claim 26, wherein the compound is a compound of formula (IIb): 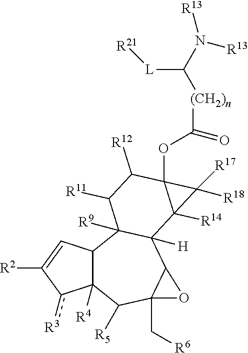
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is 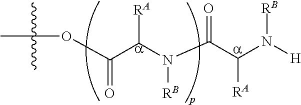
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

31. The method of claim 28, wherein the compound is a compound of formula (IIc): 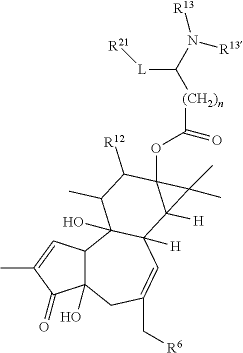
or a pharmaceutically acceptable salt, tautomer, or stereoisomer thereof;
wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 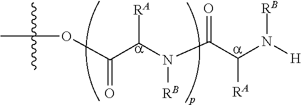
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

32. The method of claim 26, wherein the compound is a compound of formula (III): 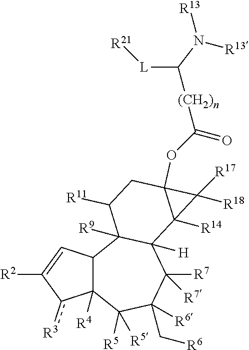
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is 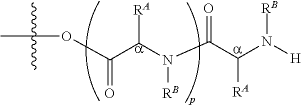
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷ is H, or R⁶′ and R⁷ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

33. The method of claim 32, wherein the compound is a compound of formula (III′): 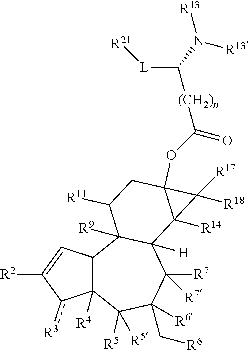
or a pharmaceutically acceptable salt, tautomer, or stereoisomer thereof.

34. The method of claim 32, wherein the compound is a compound of formula (IIIa): 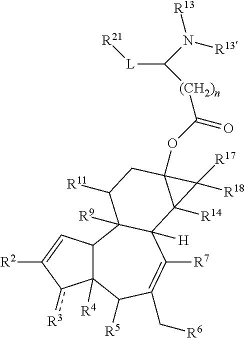
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 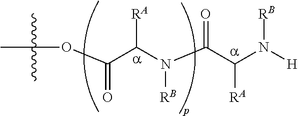
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

35. The method of claim 32, wherein the compound is a compound of formula (IIIb): 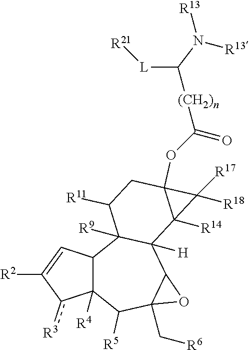
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; or
R⁶ is 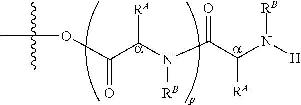
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R⁹ is OR^{e}, wherein R^{e} is H, C₁-C₆alkyl, or aryl;
R¹¹ is C₁-C₄alkyl;
R¹³ and R^{13′} are each independently H or C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

36. The method of claim 32, wherein the compound is a compound of formula (IIIc): 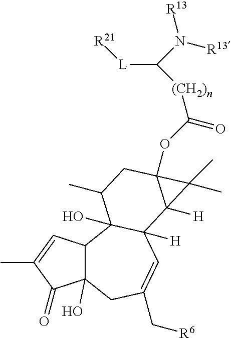
or a pharmaceutically acceptable salt, tautomer, or stereoisomer thereof;
wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 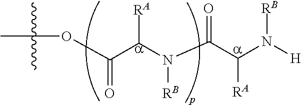
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
R¹³ and R^{13′} are each independently H or C₁-C₄alkyl;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion;
J¹ is selected from OH, CN, halo, C₁-C₄alkyl, and haloC₁-C₄alkyl; and
n is 0 or 1.

37. The method of claim 36, wherein the compound is a compound of formula (IIIe): 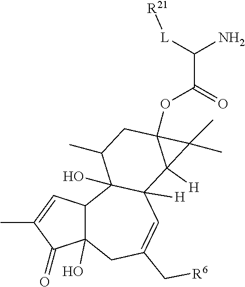
or a pharmaceutically acceptable salt, tautomer, or stereoisomer thereof;
wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; or
R⁶ is 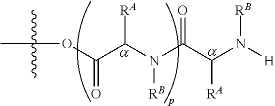
wherein,
each occurrence of R^{A} is independently selected from a side chain of a natural or non-natural amino acid, wherein each occurrence of R^{A} is same or different;
each occurrence of R^{B} is independently H, or R^{B} together with the adjacent R^{A} and the N atom to which it is attached form a heterocyclic ring of a natural or non-natural amino acid, wherein each occurrence of R^{B} is same or different; and
p is 0, 1, or 2;
L is absent, C₁-C₁₂alkylene, or C₂-C₁₂alkenylene, wherein the C₁-C₁₂alkylene or C₂-C₁₂alkenylene is optionally substituted with C₁-C₄alkyl;
R²¹ is H, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹, and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl, or when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
R^{k} is H or M⁺ counterion; and
J¹ is selected from OH, CN, halo, C₁-C₄alkyl, and haloC₁-C₄alkyl.

38. The method of any one of claims 23-37, wherein R²¹ is C₃-C₇cycloalkyl, and wherein the C₃-C₇cycloalkyl is optionally substituted with 1 to 3 of J¹.

39. The method of claim 38, wherein the C₃-C₇cycloalkyl is selected from the group consisting of cyclobutyl, cyclopentyl, cyclohexyl, and cycloheptyl, wherein the C₃-C₇cycloalkyl is optionally substituted with 1 to 3 of J¹.

40. The method of any one of claims 23-37, wherein R²¹ is heterocyclyl, wherein the heterocyclyl is optionally substituted with 1 to 3 of J¹.

41. The method of claim 40, wherein the heterocyclyl is selected from the group consisting of oxiranyl, oxetanyl, azetidynyl, oxazolyl, thiazolidinyl, thiazolyl, morpholinyl, pyrrolidinonyl, pyrrolidinyl, piperidinyl, piperazinyl, 2,3-dihydrofuranyl, dihydropyranyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and azapanyl, wherein the heterocyclyl is optionally substituted with 1 to 3 of J¹.

42. The method of any one of claims 23-37, wherein
R²¹ is aryl, wherein the aryl is optionally substituted with 1 to 3 of J¹.

43. The method of claim 42, wherein
R²¹ is a phenyl or naphthyl, wherein the phenyl or naphthyl is optionally substituted with 1 to 3 of J¹.

44. The method of any one of claims 23-37, wherein
R²¹ is heteroaryl, wherein the heteroaryl is optionally substituted with 1 to 3 of J¹.

45. The method of claim 44 wherein the heteroaryl is selected from the group consisting of pyrrolyl, pyrazolyl, imidazolyl, pyrazinyl, oxazolyl, isoxazolyl, thiazolyl, furyl, thienyl, pyridyl, pyrimidyl, benzoxazolyl, benzothiazolyl, purinyl, benzimidazolyl, indolyl, isoquinolyl, quinoxalinyl, and quinolyl, wherein the heteroaryl is optionally substituted with 1 to 3 of J¹.

46. The method of any one of claims 23-37, wherein
R²¹ is adamantyl, wherein the adamantyl is optionally substituted with OH, halo, or C₁-C₄alkyl.

47. The method of any one of claims 23-37, wherein
R²¹ is spiroC₅-C₁₂ cycloalkyl, wherein the spiroC₅-C₁₂ cycloalkyl has 0-2 carbon atoms replaced with 0-2 heteroatoms selected from N, O and S, and is optionally substituted with 1 to 3 of J¹, or when an N atom is present an N-protecting group.

48. The method of any one of claims 23-37, wherein
R²¹ is 5 to 12 membered bridged bicyclyl, wherein the bridged bicyclyl has 0-2 carbon atoms replaced with 0-2 heteroatoms selected from N, O or S, and is optionally substituted with 1 to 3 of J¹, or when an N atom is present an N-protecting group.

49. The method of any one of claims 23-37, wherein R²¹ is selected from: 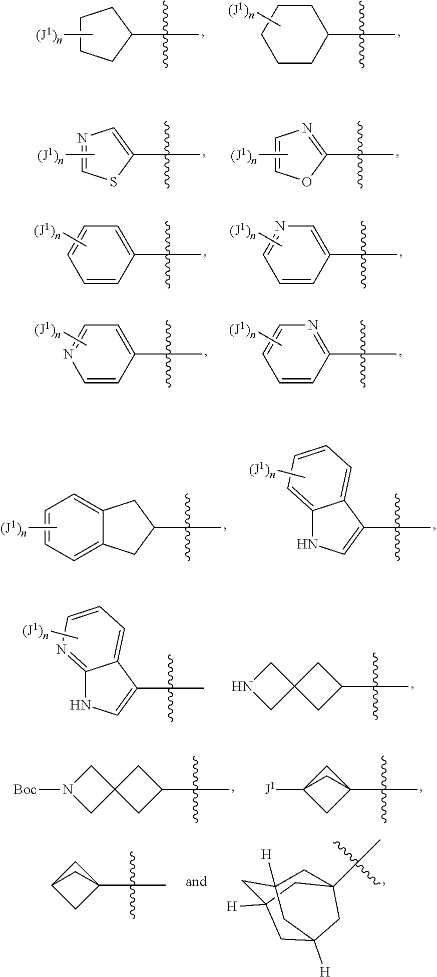
wherein J¹ is OH, CN, halo, C₁-C₄alkyl, and haloC₁-C₄alkyl, and n is 0-3.

50. The method of any one of claims 23-49, wherein L is C₁-C₆alkylene, C₃-C₆alkylene or C₃-C₁₂alkylene.

51. The method of any one of claims 23-49, wherein L is C₁-C₆alkenylene, C₃-C₆alkenylene or C₃-C₁₂alkenylene.

52. The method of any one of claims 23-51, wherein n is 0.

53. The method of any one of claims 23-52, wherein R⁶ is OH.

54. The method of any one of claims 23-52, wherein R⁶ is —OP(O)(OR^{b}′)₂, wherein each R^{b}′ is independently H, C₁-C₆alkyl, C₂-C₆alkenyl, C₀-C₆alkylaryl, or C₀-C₆alkylheteroaryl.

55. The method of any one of claims 23-52, wherein R⁶ is —OC(O)R^{c}, wherein R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c} together with the N atom form a 5 to 7 membered heterocyclyl containing 1 to 3 heteroatoms selected from N, O, and S; and R^{k} is H or a M⁺ counterion.

56. The method of any one of claims 23-52, wherein:
R⁶ is 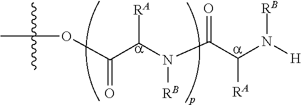
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

57. The method of claim 56, wherein
each R^{A} is independently hydrogen (glycine), methyl (alanine), propan-2-yl (valine), propan-1-y1 (norvaline), 2-methylpropan-1-y1 (leucine), 1-methylpropan-1-yl (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each R^{B} is H, or R^{B} together with the adjacent R_{A} and the N atom form a prolyl side chain: 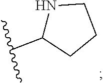 [SMILES: *C1CCCN1]
and
p is 0, 1 or 2.

58. The method of claim 57, wherein:
each R^{A} is independently methyl (alanine), propan-2-y1 (valine), 2-methylpropan-1-yl (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-yl (arginine), benzyl (phenylalanine), or 4-aminobutan-1-y1 (lysine);
R^{B} is H; and
p 0, 1, or 2.

59. The method of claim 57, wherein
each R^{A} is independently propan-2-yl (valine), 2-methylpropan-1-yl (leucine), carboxymethyl (aspartic acid), benzyl (phenylalanine), or 4-aminobutan-1-y1 (lysine);
each R^{B} is H; and
p is 0, 1, or 2.

60. The method of claim 57, wherein p is 0.

61. The method of claim 57, wherein p is 1.

62. The method of claim 57, wherein p is 1;
first of R^{A} is propan-2-y1 (valine) and second of R^{A} is propan-2-y1 (valine); and each of R^{B} is H (dipeptide Val-Val); or
first of R^{A} is 2-methylpropan-1-y1 (leucine), and second of R^{A} is 2-methylpropan-1-yl (leucine); and each of R^{B} is H (dipeptide Leu-Leu); or
first of R^{A} is methyl (alanine) and second of R^{A} is methyl (alanine); and each of R^{B} is H (dipeptide Ala-Ala); or
first of R^{A} is 4-aminobutan-1-y1 (lysine); second of R^{A} is 4-aminobutan-1-y1 (lysine); and each of R^{B} is H (dipeptide Lys-Lys); or
first of R^{A} is hydrogen; second of R^{A} is 4-aminobutan-1-yl, and each of R^{B} is H (dipeptide Gly-Lys).

63. The method of any one of claims 56-62, wherein each of the α-carbon of the amino acid other than glycine is in the L or D configuration.

64. The method of claim 23, wherein the compound is selected from the group consisting of the compounds of Table 1.

65. The method of claim 64, wherein the substituent on the C20 carbon atom of the compound is replaced with —OP(O)(OR^{b}′)₂, wherein each R^{b}′ is independently H, C₁-C₆alkyl, C₂-C₆alkenyl, C₀-C₆alkylaryl, or C₀-C₆alkylheteroaryl.

66. The method of claim 64, wherein the substituent on the C20 carbon atom of the compound is replaced with —OC(O)R^{c}, wherein R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; and R^{k} is H or a M⁺ counterion.

67. The method of claim 64, wherein the substituent on the C₂₀ carbon atom of the compound is, where appropriate, replaced with 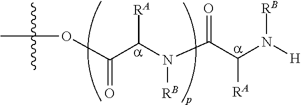
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

68. The method of claim 67, wherein
each R^{A} is independently hydrogen (glycine), methyl (alanine), propan-2-yl (valine), propan-1-y1 (norvaline), 2-methylpropan-1-y1 (leucine), 1-methylpropan-1-yl (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each R^{B} is H, or R^{B} together with the adjacent R_{A} and the N atom form a prolyl side chain: 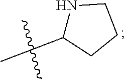 [SMILES: *C1CCCN1]
and
p is 0, 1 or 2.

69. The method of claim 68, wherein:
each R^{A} is independently methyl (alanine), propan-2-y1 (valine), 2-methylpropan-1-yl (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-yl (arginine), benzyl (phenylalanine), or 4-aminobutan-1-y1 (lysine);
R^{B} is H; and
p 0, 1, or 2.

70. The method of claim 68, wherein
each R^{A} is independently propan-2-yl (valine), 2-methylpropan-1-yl (leucine), carboxymethyl (aspartic acid), benzyl (phenylalanine), or 4-aminobutan-1-y1 (lysine);
each R^{B} is H; and
p is 0, 1, or 2.

71. The method of claim 68, wherein p is 0.

72. The method of claim 68, wherein p is 1.

73. The method of claim 68 wherein p is 1;
first of R^{A} is propan-2-y1 (valine) and second of R^{A} is propan-2-y1 (valine); and each of R^{B} is H (dipeptide Val-Val); or
first of R^{A} is 2-methylpropan-1-y1 (leucine), and second of R^{A} is 2-methylpropan-1-y1 (leucine); and each of R^{B} is H (dipeptide Leu-Leu); or
first of R^{A} is methyl (alanine) and second of R^{A} is methyl (alanine); and each of R^{B} is H (dipeptide Ala-Ala); or
first of R^{A} is 4-aminobutan-1-y1 (lysine); second of R^{A} is 4-aminobutan-1-y1 (lysine); and each of R^{B} is H (dipeptide Lys-Lys); or
first of R^{A} is hydrogen; second of R^{A} is 4-aminobutan-1-y1, and each of R^{B} is H (dipeptide Gly-Lys).

74. The method of any one of claims 67-73, wherein each of the α-carbon of the amino acid other than glycine is in the L or D configuration.

75. The method of any one of claims 1-22, wherein the compound is a compound of formula (IV): 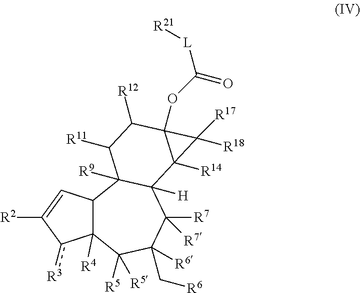
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 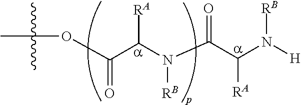
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

76. The method of claim 75, wherein the compound is a compound of formula (IVa): 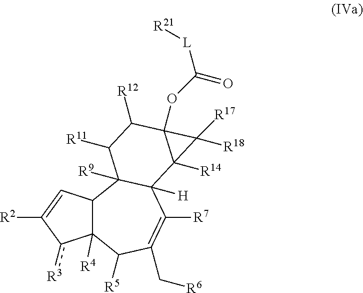
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, —OP(O)(OR^{b}′)₂, halo, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is 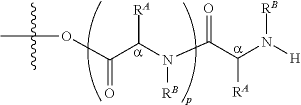
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

77. The method of claim 75, wherein the compound is a compound of formula (IVb) 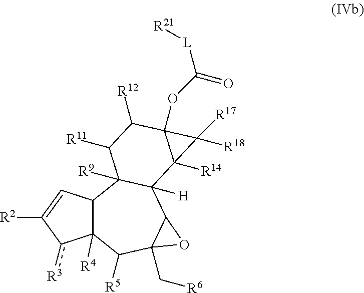
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; or
R⁶ is 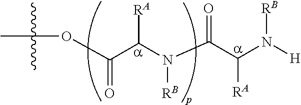
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J;
J¹ is selected from OH, CN, halo, C₁-C₄alkyl, and haloC₁-C₄alkyl; and
R^{k} is H or M⁺ counterion.

78. The compound of claim 76, having the structure formula (IVc): 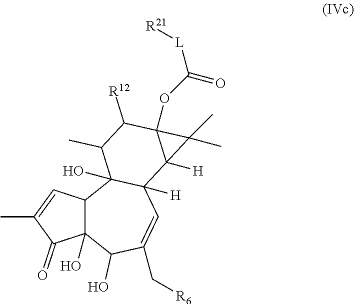
or a pharmaceutically acceptable salt, tautomer, or stereoisomer thereof;
wherein
R⁶ is OH, halo, or —OC(O)R^{c}, wherein R^{c} is —C₁-C₆alkyl, —C₁-C₆alkyl-(NR^{c1})₂ or —C₁-C₆alkylC(O)OR^{k}, R^{c1} is H, C₁-C₆alkyl, or two R^{c} together with the N atom form a 5 to 7 membered heterocyclyl containing 1 to 3 heteroatoms selected from N, O, and S; or
R⁶ is 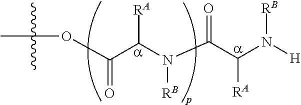
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂ cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

79. The compound of claim 76, having the structure of formula (IVd): 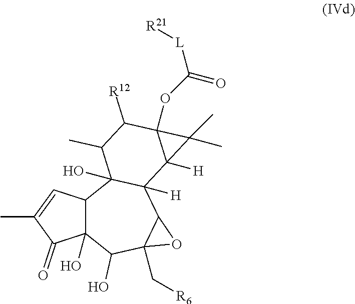
or a pharmaceutically acceptable salt, tautomer, or stereoisomer thereof;
wherein
R⁶ is OH, halo, or —OC(O)R^{c}, wherein R^{c} is —C₁-C₆alkyl, —C₁-C₆alkyl-(NR^{c1})₂ or —C₁-C₆alkylC(O)OR^{k}, R^{c1} is H, C₁-C₆alkyl, or two R^{c1} together with the N atom form a 5 to 7 membered heterocyclyl containing 1 to 3 heteroatoms selected from N, O, and S; or
R⁶ is 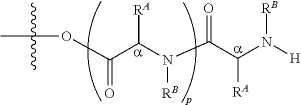
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R¹² is H, —OH, —OC(O)R^{f}, wherein R^{f} is C₁-C₁₂alkyl, C₂-C₁₂alkenyl, —C₀-C₁₂aliphatic-C₃-C₇cycloalkyl, —C₀-C₁₂aliphatic-heterocycloalkyl, —C₀-C₁₂aliphatic-aryl, or —C₀-C₁₂aliphatic-heteroaryl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

80. The compound of claim 76, having the structure of formula IVe or IVf, or an amino acid prodrug thereof: 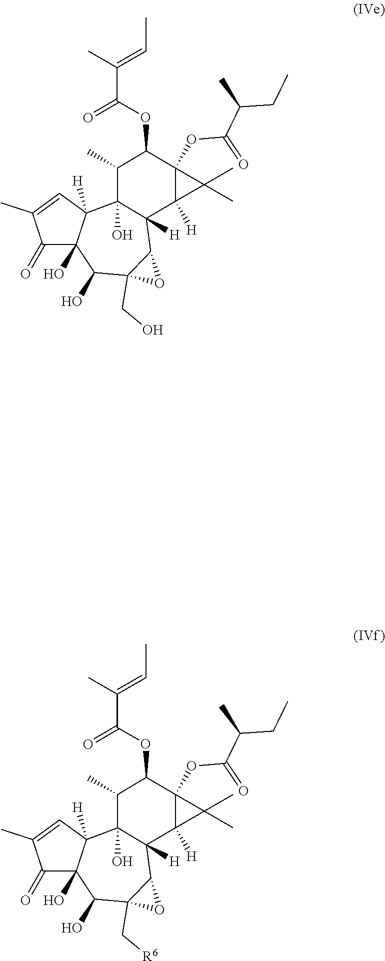
wherein for the structural formula IVf, R⁶ is 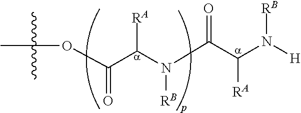
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

81. The method of claim 76, wherein the compound is a compound of formula (V): 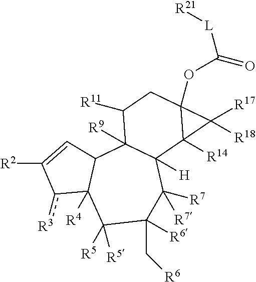
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁵′ and R⁶′ are each independently H or OH, or R⁵′ and R⁶′ form a bond or are bonded to a common O atom to form an epoxide ring as permitted by valency;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 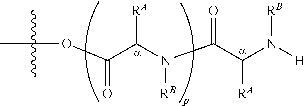
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁶′ is H or OH, R⁷′ is H, or R⁶′ and R⁷′ form a bond or are bonded to a common O atom to form an epoxide ring, as permitted by valency;
R⁷ is H or OH;
R⁹ is OR^{e}, wherein R^{e} is H, C₁-C₆alkyl, or aryl;
R¹¹ is C₁-C₄alkyl;
R¹⁴ is H or OR⁹; wherein R⁹ is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

82. The method of claim 75, wherein the compound is a compound of formula (Va): 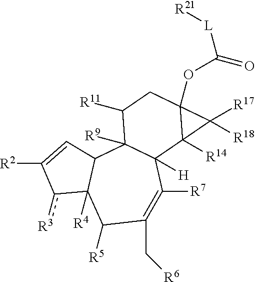
or a pharmaceutically acceptable salt, tautomer, or stereoisomer thereof;
wherein
or an enantiomer or pharmaceutically acceptable salt thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c1} together with the N atom form a 5 to 7 membered heterocyclyl containing 1 to 3 heteroatoms selected from N, O, and S; or
R⁶ is 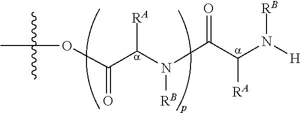
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁷ is H or OH;
R⁹ is OR^{e}, wherein R^{e} is H, C₁-C₆alkyl, or aryl;
R¹¹ is C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

83. The method of claim 75, wherein the compound is a compound of formula (Vb): 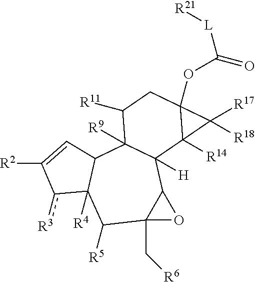
or a pharmaceutically acceptable salt, tautomer, or stereoisomer thereof;
wherein
R² is a C₁-C₄alkyl;
R³ is O double bonded to the ring carbon when (- - -) is a bond, or —OR^{a}; wherein R^{a} is H or —C(O)R^{a1}, wherein R^{a1} is C₁-C₆alkyl, C₂-C₆alkenyl, C₀-C₆alkylaryl, or C₀-C₆alkylheteroaryl;
R⁴ and R⁵ are each independently H or —OR^{b}, wherein R^{b} is H, C₁-C₆alkyl, C₂-C₆alkenyl, C₀-C₆alkylaryl, or C₀-C₆alkylheteroaryl;
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; or
R⁶ is 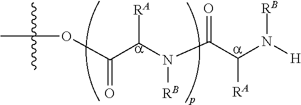
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
R⁹ is OR^{e}, wherein R^{e} is H, C₁-C₆alkyl, or aryl;
R¹¹ is C₁-C₄alkyl;
R¹⁴ is H or OR^{g}; wherein R^{g} is H or C₁-C₆alkyl;
R¹⁷ and R¹⁸ are each independently C₁-C₄alkyl or C₁-C₄alkyl-OR^{h}, wherein R^{h} is H or C₁-C₆alkyl;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

84. The method of claim 75, wherein the compound is a compound of formula (Vc): 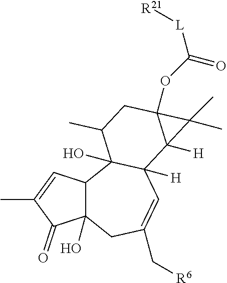
or a pharmaceutically acceptable salt, tautomer, or stereoisomer thereof;
wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 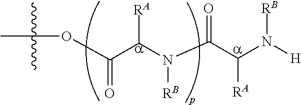
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J¹; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

85. The method of claim 75, wherein the compound is a compound of formula (Vd): 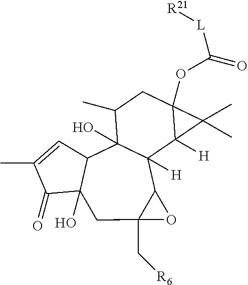
or a pharmaceutically acceptable salt, tautomer, or stereoisomer thereof;
wherein
R⁶ is OH, halo, —OP(O)(OR^{b}′)₂, or —OC(O)R^{c}, wherein each R^{b}′ is independently H, C₁-C₆alkyl, C₂-C₆alkenyl, C₀-C₆alkylaryl, or C₀-C₆alkylheteroaryl; R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R¹ together with the N atom form a 5 to 7 membered heterocyclyl containing 1 to 3 heteroatoms selected from N, O, and S; or
R⁶ is 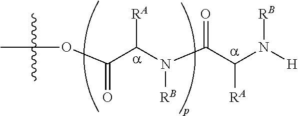
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2;
L is C₀-C₆alkylarylene, C₀-C₆alkylheteroarylene, C₀-C₆alkylC₃-C₇cycloalkylene, C₁-C₁₂alkylene or C₂-C₁₂alkenylene, wherein the C₁-C₁₂alkylene or C₂-C₁₂alkenylene is optionally substituted with OH or C₁-C₄alkyl; and
R²¹ is H, —OH, —SH, —S(O)₂R^{j}, —SR^{j}, —N(R^{j})₂, —Si(R^{j})₃, C₃-C₇cycloalkyl, heterocyclyl, aryl, heteroaryl, spiroC₅-C₁₂cycloalkyl, 5 to 12 membered bridged bicyclyl, adamantyl, or —C(O)OR^{k}, wherein the C₃-C₇cycloalkyl, heterocyclyl, aryl, heteroaryl, spiroC₅-C₁₂ cycloalkyl, bridged bicyclyl, or adamantyl is optionally substituted with 1 to 3 of J; and wherein optionally 1 to 2 carbon atoms of the spiroC₅-C₁₂cycloalkyl or bridged bicyclyl is replaced with a heteroatom selected from N, O and S, and optionally substituted with C₁-C₄alkyl or, when an N atom is present an N-protecting group;
each R^{j} is independently C₁-C₆alkyl, C₂-C₆alkenyl, C₀-C₆alkylC₃-C₇cycloalkyl, C₀-C₆alkylheterocyclyl, C₀-C₆alkylaryl, or C₀-C₆alkylheteroaryl, wherein the C₃-C₇cycloalkyl, heterocyclyl, alkylaryl, or heteroaryl is optionally substituted with 1 to 3 of J¹;
J¹ is selected from OH, CN, halo, C₁-C₄alkyl, and haloC₁-C₄alkyl; and
R^{k} is H or M⁺ counterion.

86. The method of any one of claims 75-85, wherein
R²¹ is C₃-C₇cycloalkyl, wherein the C₃-C₇cycloalkyl is optionally substituted with 1 to 3 of J¹.

87. The method of claim 86, wherein the C₃-C₇cycloalkyl is selected from the group consisting of cyclobutyl, cyclopentyl, cyclohexyl, and cycloheptyl, wherein the C₃-C₇cycloalkyl is optionally substituted with 1 to 3 of J¹.

88. The method of any one of claims 75-85, wherein
R²¹ is heterocyclyl, wherein the heterocyclyl is optionally substituted with 1 to 3 of J¹.

89. The method of claim 88, wherein the heterocyclyl is selected from the group consisting of oxiranyl, oxetanyl, azetidynyl, oxazolyl, thiazolidinyl, thiazolyl, morpholinyl, pyrrolidinonyl, pyrrolidinyl, piperidinyl, piperazinyl, 2,3-dihydrofuranyl, dihydropyranyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and azapanyl, wherein the heterocyclyl is optionally substituted with 1 to 3 of J¹.

90. The method of any one of claims 75-85, wherein
R²¹ is aryl, wherein the aryl is optionally substituted with 1 to 3 of J¹.

91. The method of claim 90, wherein
R²¹ is a phenyl or naphthyl, wherein the phenyl or naphthyl is optionally substituted with 1 to 3 of J¹.

92. The method of any one of claims 75-85, wherein
R²¹ is heteroaryl, wherein the heteroaryl is optionally substituted with 1 to 3 of J¹.

93. The method of claim 92, wherein the heteroaryl is selected from the group consisting of pyrrolyl, pyrazolyl, imidazolyl, pyrazinyl, oxazolyl, isoxazolyl, thiazolyl, furyl, thienyl, pyridyl, pyrimidyl, benzoxazolyl, benzothiazolyl, purinyl, benzimidazolyl, indolyl, isoquinolyl, quinoxalinyl, and quinolyl, wherein the heteroaryl is optionally substituted with 1 to 3 of J¹.

94. The method of any one of claims 75-85, wherein
R²¹ is adamantyl, wherein the adamantyl is optionally substituted with 1 to 3 of J¹.

95. The method of any one of claims 75-85, wherein
R²¹ is spiroC₅-C₁₂ cycloalkyl, wherein the spiroC₅-C₁₂ cycloalkyl has 0-2 carbon atoms replaced with 0-2 heteroatoms selected from N, O and S, and is optionally substituted with 1 to 3 of J¹, or when an N atom is present an N-protecting group.

96. The method of any one of claims 75-85, wherein
R²¹ is 5 to 12 membered bridged bicyclyl, wherein the bridged bicyclyl has 0-2 carbon atoms replaced with 0-2 heteroatoms selected from N, O or S, and is optionally substituted with 1 to 3 of J¹, or when an N atom is present an N-protecting group.

97. The method of any one of claims 75-96, wherein L is C₁-C₆alkylene, C₃-C₆alkylene or C₃-C₁₂alkyl.

98. The method of any one of claims 75-96, wherein L is C₁-C₆alkenylene, C₃-C₆alkenylene or C₃-C₁₂alkenylene.

99. The method of any one of claims 75-98, wherein R⁶ is —OH.

100. The method of any one of claims 75-98, wherein R⁶ is —OP(O)(OR^{b}′)₂, wherein each R^{b}′ is independently H, C₁-C₆alkyl, C₂-C₆alkenyl, C₀-C₆alkylaryl, or C₀-C₆alkylheteroaryl.

101. The method of any one of claims 75-98, wherein R⁶ is —OC(O)R^{c}, wherein R^{c} is —C₁-C₆alkyl, —C₁-C₆alkyl-(NR^{c1})₂ or —C₁-C₆alkylC(O)OR^{k}; R^{c} is H, C₁-C₆alkyl, or two R^{c} together with the N atom form a 5 to 7 membered heterocyclyl containing 1 to 3 heteroatoms selected from N, O, and S; and R^{k} is H or a M⁺ counterion.

102. The method of any one of claims 75-98, wherein: R⁶ is 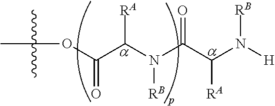
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

103. The method of claim 104, wherein
each R^{A} is independently hydrogen (glycine), methyl (alanine), propan-2-yl (valine), propan-1-y1 (norvaline), 2-methylpropan-1-y1 (leucine), 1-methylpropan-1-y1 (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-yl (ornithine), 3-guanidinopropan-1-y1 (arginine), or 3-ureido-propan-1-yl (citrulline);
each R^{B} is H, or R^{B} together with the adjacent R_{A} and the N atom form a prolyl side chain: 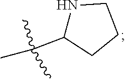 [SMILES: *C1CCCN1]
and
p is 0, 1 or 2.

104. The method of claim 103, wherein:
each R^{A} is independently methyl (alanine), propan-2-y1 (valine), 2-methylpropan-1-yl (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-yl (arginine), benzyl (phenylalanine), or 4-aminobutan-1-y1 (lysine);
R^{B} is H; and
p 0, 1, or 2.

105. The method of claim 103, wherein
each R^{A} is independently propan-2-yl (valine), 2-methylpropan-1-yl (leucine), carboxymethyl (aspartic acid), benzyl (phenylalanine), or 4-aminobutan-1-y1 (lysine);
each R^{B} is H; and
p is 0, 1, or 2.

106. The method of claim 103, wherein p is 0.

107. The method of claim 103, wherein p is 1.

108. The method of claim 103, wherein p is 1;
first of R^{A} is propan-2-y1 (valine) and second of R^{A} is propan-2-y1 (valine); and each of R^{B} is H (dipeptide Val-Val); or
first of R^{A} is 2-methylpropan-1-y1 (leucine), and second of R^{A} is 2-methylpropan-1-yl (leucine); and each of R^{B} is H (dipeptide Leu-Leu); or
first of R^{A} is methyl (alanine) and second of R^{A} is methyl (alanine); and each of R^{B} is H (dipeptide Ala-Ala); or
first of R^{A} is 4-aminobutan-1-yl (lysine); second of R^{A} is 4-aminobutan-1-yl (lysine); and each of R^{B} is H (dipeptide Lys-Lys); or
first of R^{A} is hydrogen; second of R^{A} is 4-aminobutan-1-yl, and each of R^{B} is H (dipeptide Gly-Lys).

109. The method of any one of claims 102-108, wherein each of the α-carbon of the amino acid other than glycine is in the L or D configuration.

110. The method of claim 75, wherein the compound is selected from the group consisting of the compounds of Table 2.

111. The method of claim 110, wherein the substituent on the C₂₀ carbon atom of the compound is replaced with —OP(O)(OR^{b}′)₂, wherein each R^{b}′ is independently H, C₁-C₆alkyl, C₂-C₆alkenyl, C₀-C₆alkylaryl, or C₀-C₆alkylheteroaryl.

112. The method of claim 110, wherein the substituent on the C₂₀ carbon atom of the compound is replaced with —OC(O)R^{c}, wherein R^{c} is —C₁-C₆alkyl, —C₁-C₆alkyl-(NR^{c1})₂ or —C₁-C₆alkylC(O)OR^{k}; R^{c1} is H, C₁-C₆alkyl, or two R^{c} together with the N atom form a 5 to 7 membered heterocyclyl containing 1 to 3 heteroatoms selected from N, O, and S; and R^{k} is H or a M⁺ counterion.

113. The method of claim 110, wherein the substituent on the C₂₀ carbon atom of the compound, where appropriate, is substituted with 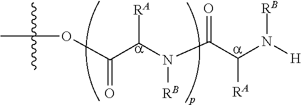
wherein,
each occurrence of R^{A} is independently selected from a side chain of a natural or non-natural amino acid, wherein each occurrence of R^{A} is same or different;
each occurrence of R^{B} is independently H, or R^{B} together with R^{A} and the N atom to which it is attached form a heterocyclic ring of a natural or non-natural amino acid, wherein each occurrence of R^{B} is same or different; and
p is 0, 1, or 2.

114. The method of claim 113, wherein
each R^{A} is independently hydrogen (glycine), methyl (alanine), propan-2-yl (valine), propan-1-y1 (norvaline), 2-methylpropan-1-y1 (leucine), 1-methylpropan-1-y1 (isoleucine), butan-1-y1 (norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-y1 (lysine), 4-amino-3-hydroxybutan-1-y1 (hydroxylysine), 3-aminopropan-1-y1 (ornithine), 3-guanidinopropan-1-y1 (arginine), or 3-ureido-propan-1-yl (citrulline);
each R^{B} is H, or R^{B} together with the adjacent R_{A} and the N atom form a prolyl side chain: 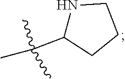 [SMILES: *C1CCCN1]
and
p is 0, 1 or 2.

115. The method of claim 113, wherein:
each R^{A} is independently methyl (alanine), propan-2-y1 (valine), 2-methylpropan-1-yl (leucine), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-y1 (lysine), carboxymethyl (aspartic acid), 3-guanidinopropan-1-yl (arginine), benzyl (phenylalanine), or 4-aminobutan-1-y1 (lysine);
R^{B} is H; and
p 0, 1, or 2.

116. The method of claim 113, wherein
each R^{A} is independently propan-2-yl (valine), 2-methylpropan-1-yl (leucine), carboxymethyl (aspartic acid), benzyl (phenylalanine), or 4-aminobutan-1-y1 (lysine);
each R^{B} is H; and
p is 0, 1, or 2.

117. The method of claim 113, wherein p is 0.

118. The method of claim 113, wherein p is 1.

119. The method of claim 113, wherein p is 1;
first of R^{A} is propan-2-y1 (valine) and second of R^{A} is propan-2-y1 (valine); and each of R^{B} is H (dipeptide Val-Val); or
first of R^{A} is 2-methylpropan-1-y1 (leucine), and second of R^{A} is 2-methylpropan-1-y1 (leucine); and each of R^{B} is H (dipeptide Leu-Leu); or
first of R^{A} is methyl (alanine) and second of R^{A} is methyl (alanine); and each of R^{B} is H (dipeptide Ala-Ala); or
first of R^{A} is 4-aminobutan-1-y1 (lysine); second of R^{A} is 4-aminobutan-1-y1 (lysine); and each of R^{B} is H (dipeptide Lys-Lys); or
first of R^{A} is hydrogen; second of R^{A} is 4-aminobutan-1-y1, and each of R^{B} is H (dipeptide Gly-Lys).

120. The method of any one of claims 113-119, wherein each of the α-carbon of the amino acid other than glycine is in the L or D configuration.
